(19) United States
(12) Patent Application Publication
(10) Pub. No.: US 2008/0154286 A1
(43) Pub. Date: Jun. 26, 2008

(54) Systems and Methods for Treating Septal Defects with Capture Devices and Other Devices

(76) Inventors: Ryan Abbott, San Jose, CA (US); W. Martin Belef, San Jose, CA (US); Dean Carson, Mountain View, CA (US); Ronald J. Jabba, Redwood City, CA (US); David Moore, San Carlos, CA (US); James Nielsen, San Francisco, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/614,814

(22) Filed: Dec. 21, 2006

(51) Int. Cl. A61B 17/04

(52) U.S. Cl. 606/139; 606/228

(57) ABSTRACT

Systems, devices and methods for treating internal tissue defects, such as septal defects, are provided. An exemplary method of treating an internal tissue defect, specifically a method of closing a patent foramen ovale (PFO), can include passing a closure element from a right atrium through a septal wall in a first location and into the left atrium. A capture device, which can be configured as a snare-like device, can be used to capture the closure element in the left atrium and pull the closure element back through the septal wall in a different, second location, such that the closure element is routed over the PFO. The closure element can then be anchored and/or locked against the septal wall such that the PFO is at least partially closed.

### FIELD OF THE INVENTION

The present invention relates generally to systems and methods for treating internal tissue defects, such as septal defects, with capture devices.

### BACKGROUND OF THE INVENTION

By nature of their location, the treatment of internal tissue defects is inherently difficult. Access to a defect through invasive surgery introduces a high level of risk that can result in serious complications for the subject. Access to the defect remotely with a catheter or equivalent device is less risky, but treatment of the defect itself is made more difficult given the limited physical abilities of the catheter. The difficulty in accessing and treating tissue defects is compounded when the defect is found in or near a vital organ. For instance, a patent foramen ovale (“PFO”) is a serious septal defect that can occur between the left and right atria of the heart and a patent ductus arteriosus (“PDA”) is an abnormal shunt between the aorta and pulmonary artery.

During development of a fetus in utero, oxygen is transferred from maternal blood to fetal blood through complex interactions between the developing fetal vasculature and the mother's placenta. During this process, blood is not oxygenated within the fetal lungs. In fact, most of the fetus' circulation is shunted away from the lungs through specialized vessels and foramens that are open during fetal life, but typically will close shortly after birth. Occasionally, however, these foramen fail to close and create hemodynamic problems, which, in extreme cases, can prove fatal. During fetal life, an opening called the foramen ovale allows blood to bypass the lungs and pass directly from the right atrium to the left atrium. Thus, blood that is oxygenated via gas exchange with the placenta may travel through the vena cava into the right atrium, through the foramen ovale into the left atrium, and from there into the left ventricle for delivery to the fetal systemic circulation. After birth, with pulmonary circulation established, the increased left atrial blood flow and pressure causes the functional closure of the foramen ovale and, as the heart continues to develop, this closure allows the foramen ovale to grow completely sealed.

In some cases, however, the foramen ovale fails to close entirely. This condition, known as a PFO, can allow blood to continue to shunt between the left and right atria of the heart throughout the adult life of the individual. A PFO can pose serious health risks for the individual, including strokes and migraines. The presence of PFO's have been implicated as a possible contributing factor in the pathogenesis of migraines. Two current hypothesis that link PFO's with migraine include the transit of vasoactive substances or thrombus/emboli from the venous circulation directly into the left atrium without passing through the lungs where they would normally be deactivated or filtered respectively. Other diseases that have been associated with PFO's (and which could benefit from PFO closure) include but are not limited to depression and affective disorders, personality and anxiety disorders, pain, stroke, transient ischemic attacks (TIA), dementia, epilepsy, and sleep disorders.

Still other septal defects can occur between the various chambers of the heart, such as atrial-septal defects (ASD's), ventricular-septal defects (VSD's), and the like. To treat these defects as well as PFO's, open heart surgery can be performed to ligate or patch the defect closed. Alternatively, catheter-based procedures have been developed that require introducing umbrella or disc-like devices into the heart. These devices include opposing expandable structures connected by a hub or waist. Generally, in an attempt to close the defect, the device is inserted through the natural opening of the defect and the expandable structures are deployed on either side of the septum to secure the tissue surrounding the defect between the umbrella or disc-like structure.

These devices suffer from numerous shortcomings. For instance, these devices typically involve frame structures that often support membranes, either of which may fail during the life of the subject, thereby introducing the risk that the defect may reopen or that portions of the device could be released within the subject's heart. These devices can fail to form a perfect seal of the septal defect, allowing blood to continue to shunt through the defect. Also, the size and expansive nature of these devices makes safe withdrawal from the subject difficult in instances where withdrawal becomes necessary. The presence of these devices within the heart typically requires the subject to use anti-coagulant drugs for prolonged periods of time, thereby introducing additional health risks to the subject. Furthermore, these devices can come into contact with other portions of the heart tissue and can cause undesirable side effects such as an arrhythmia, local tissue damage, and perforation.

Accordingly, improved devices, systems and methods for treating and closing internal tissue defects within the heart are needed.

### SUMMARY

Improved devices, systems and methods for treating internal tissue defects, such as septal defects and the like, are provided in this section by the way of exemplary embodiments. These embodiments are examples only and are not intended to limit the invention.

Provided herein are embodiments of systems, devices and methods for treating, and preferably closing, septal defects and the like. These systems, devices and methods generally make use of one or more piercing elements used to create an opening in a part of a septal wall. Various devices can be introduced through the opening, including closure devices, capture devices, tubular members, and lock devices, to facilitate treatment of the septal defect. In one exemplary embodiment, a first end of a suture-like closure element is advanced through a first opening in a septal wall having a PFO, while a capture device is advanced through a separate opening in the septal wall into the same atrial chamber. There, the capture device is used to capture the first end of the closure element and retrieve the element back through the septal wall to the opposite atrial chamber. Both ends of the closure element can then be secured in the opposite atrial chamber to at least partially close the PFO.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention is not limited to require the details of the example embodiments.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1 is a block diagram depicting an exemplary embodiment of a treatment system for treating internal tissue defects.

FIG. 2A is an exterior/interior view depicting an example human heart with a portion of the inferior vena cava and the superior vena cava connected thereto.

FIG. 2B-C are enlarged views of a septal wall taken from FIG. 2A depicting a PFO region.

FIG. 2D is a cross-sectional view depicting a PFO region taken along line 2D-2D of FIGS. 2B-C.

FIG. 2E is a cross-sectional view depicting an example PFO region taken along line 2E-2E of FIG. 2D.

FIG. 3A is an exterior/interior view depicting an example human heart with an exemplary embodiment of the treatment system located therein.

FIG. 3B is a perspective view depicting an exemplary embodiment of a delivery device in proximity with a septal wall.

FIGS. 3C-E are perspective views depicting an exemplary embodiment of the treatment system using an “off-axis” delivery configuration.

FIG. 3F is a partial cross-sectional view depicting an exemplary embodiment of the treatment system in position within a septal wall.

FIGS. 3G-H are perspective views depicting additional exemplary embodiments of the treatment system using an “off-axis” delivery configuration.

FIG. 3I is an end on view of the septal wall taken from the right atrium showing another exemplary embodiment of the delivery device during an exemplary closure procedure.

FIGS. 4A-4B are perspective views depicting additional exemplary embodiments of the treatment system using an “off-axis” delivery configuration.

FIG. 4C-F are top down views depicting additional exemplary embodiments of the treatment system.

FIG. 5A-B are partial cross-sectional views depicting an exemplary embodiment of the delivery device during an exemplary closure procedure.

FIG. 5C is an end on view from the left atrium depicting an exemplary embodiment of the delivery device during an exemplary closure procedure.

FIG. 5D is a side view depicting an exemplary embodiment of a needle.

FIG. 5E is a frontal view depicting another exemplary embodiment of the delivery device.

FIG. 5F is an end on view depicting another exemplary embodiment of the delivery device during an exemplary closure procedure.

FIG. 5G-P are partial cross-sectional views depicting additional exemplary embodiments of the delivery device during exemplary closure procedures.

FIGS. 5Q-R are perspective views of a septal wall depicting exemplary locations where portions of the delivery device can be inserted.

FIG. 5S-T are partial cross-sectional views depicting additional exemplary embodiments where the needles are inserted through the septal wall at angles.

FIG. 5U is a radial cross-sectional view depicting another exemplary embodiment of the treatment system.

FIGS. 6A-K are partial cross-sectional views depicting additional exemplary embodiments of the delivery device during exemplary closure procedures.

FIG. 6L-M are side views depicting additional exemplary embodiments of the treatment system during exemplary closure procedures.

FIG. 6N is a partial cross-sectional view depicting another exemplary embodiment of the delivery device during an exemplary closure procedure.

FIG. 6O is a cross-sectional view depicting another exemplary embodiment of the delivery device.

FIGS. 7A-8D are partial cross-sectional views depicting additional exemplary embodiments of the delivery device during exemplary closure procedures.

FIGS. 8E-F are perspective views depicting exemplary embodiments of the capture device.

FIGS. 9A-9D are partial cross-sectional views depicting additional exemplary embodiments of the delivery device during exemplary closure procedures.

FIG. 10A is a perspective view depicting additional exemplary embodiments of the treatment system using an “off-axis” delivery configuration.

FIGS. 10B-E are partial cross-sectional views depicting additional exemplary embodiments of the delivery device during exemplary closure procedures.

FIGS. 11A-B are perspective views depicting additional exemplary embodiments of the delivery device.

FIGS. 11C-G are partial cross-sectional views depicting additional exemplary embodiments of the delivery device during exemplary closure procedures.

FIG. 12A-B are perspective views depicting another exemplary embodiment of the delivery device.

FIGS. 13-14 are partial cross-sectional views depicting additional exemplary embodiments of the delivery device.

FIGS. 15A-16 are top down views depicting exemplary embodiments of a snare-like device.

FIGS. 17A-19C are top down views depicting exemplary embodiments of a closure element.

FIG. 20A-C are partial cross-sectional views depicting additional exemplary embodiments of a closure element implanted within a septal wall.

FIG. 21A-B are perspective views depicting an exemplary embodiment of a lock device.

FIG. 21C is a top down view depicting another exemplary embodiment of a lock device.

FIG. 21D is a perspective view depicting another exemplary embodiment of a lock device.

FIG. 21E is a side view depicting another exemplary embodiment of a lock device.

FIGS. 21F-H are top down views depicting additional exemplary embodiments of the lock device.

FIGS. 21I-J are perspective views depicting additional exemplary embodiments of lock devices.

FIGS. 21K-L are perspective views depicting exemplary embodiments of a pusher member.

FIGS. 21M-N are partial cross-sectional views depicting additional exemplary embodiments of the delivery device.

FIGS. 22A-25B are perspective views depicting additional exemplary embodiments of lock devices.

FIG. 25C is a cross-sectional view depicting another exemplary embodiment of a lock device taken along line 25C-25C of FIG. 25B.

FIGS. 26A-B are flow diagrams depicting an exemplary method of using an exemplary embodiment of the treatment system.

### DETAILED DESCRIPTION

Devices, systems and methods for treating tissue defects with a suture-like closure element using a snare-like capture device are described herein, among others. For ease of discussion, these devices, systems and methods will be described with reference to treatment of a PFO defect. However, it should be understood that these devices, systems and methods can be used in treatment of any type of septal defect including ASD's, VSD's and the like, as well as PDA's, pulmonary shunts or other structural cardiac or vascular defects or non-vascular defects, and also any other tissue defect including non-septal tissue defects.

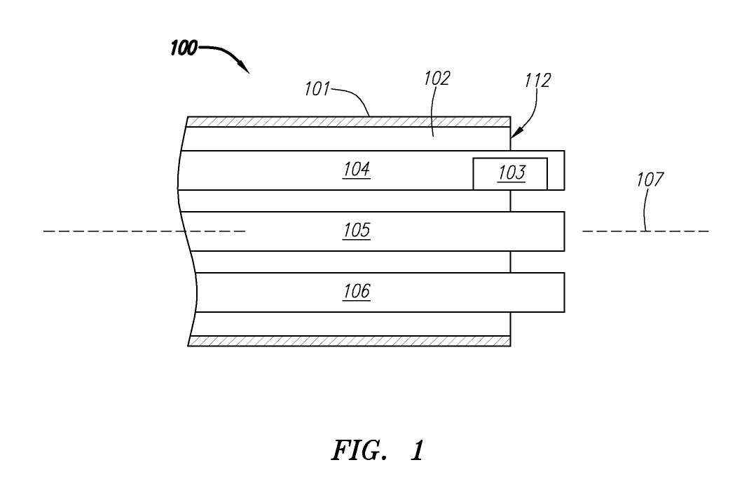

FIG. 1 is a functional block diagram depicting a distal portion of an exemplary embodiment of a septal defect treatment system 100 configured to treat and preferably close a PFO. In this embodiment, treatment system 100 includes an elongate body member 101 configured for insertion into the vasculature of a subject (human or animal) having a septal defect. Body member 101 has a longitudinal axis 107, a distal end 112 and can include one or more lumens 102, each of which can be configured for achieving multiple functions. Preferably, treatment system 100 can carry an implantable closure element 103 configured to facilitate partial or entire closure of a septal defect.

Closure element 103 is preferably configured in a suture-like manner and, to facilitate this description, will be referred to herein as suture 103. However, it should be understood that closure element 103 can have other, non-suture-like configurations and still operate in accordance with the systems, devices and methods described herein. Suture 103 can be fabricated from any material or combination of materials.

As shown in FIG. 1, treatment system 100 can include a flexible elongate delivery device 104 configured to house and deliver suture 103. Treatment system 100 can also optionally include a stabilization device 105 for stabilization of body member 101 during delivery of suture 103 and a positioning device 106 for facilitating the positioning or the centering of delivery device 104 for delivery. Although shown here as four separate components, any combination of body member 101, delivery device 104, stabilization device 105 and positioning device 106 can be integrated together to reduce the number of components to three, two or one total components in treatment system 100. A user can manipulate delivery device 104, stabilization device 105 and positioning device 106 at the proximal end of body member 101 (not shown). The use of a similar treatment systems 100, also having body members 101, delivery devices 104, stabilization devices 105 and positioning devices 106, are described in detail in co-pending U.S. patent application Ser. No. 11/175,814, filed Jul. 5, 2005 and entitled “Systems and Methods for Treating Septal Defects,” and U.S. patent application Ser. No. 11/218,794, filed Sep. 1, 2005 and entitled “Suture-based Systems and Methods for Treating Septal Defects,” both of which are fully incorporated by reference herein.

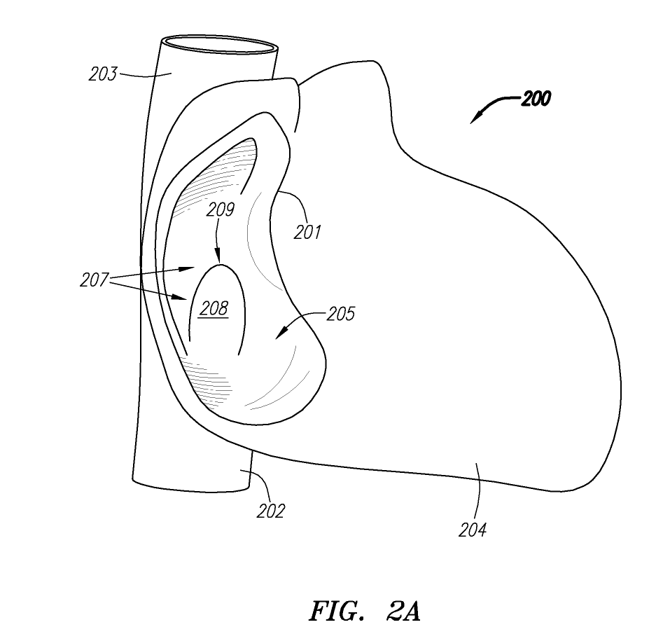

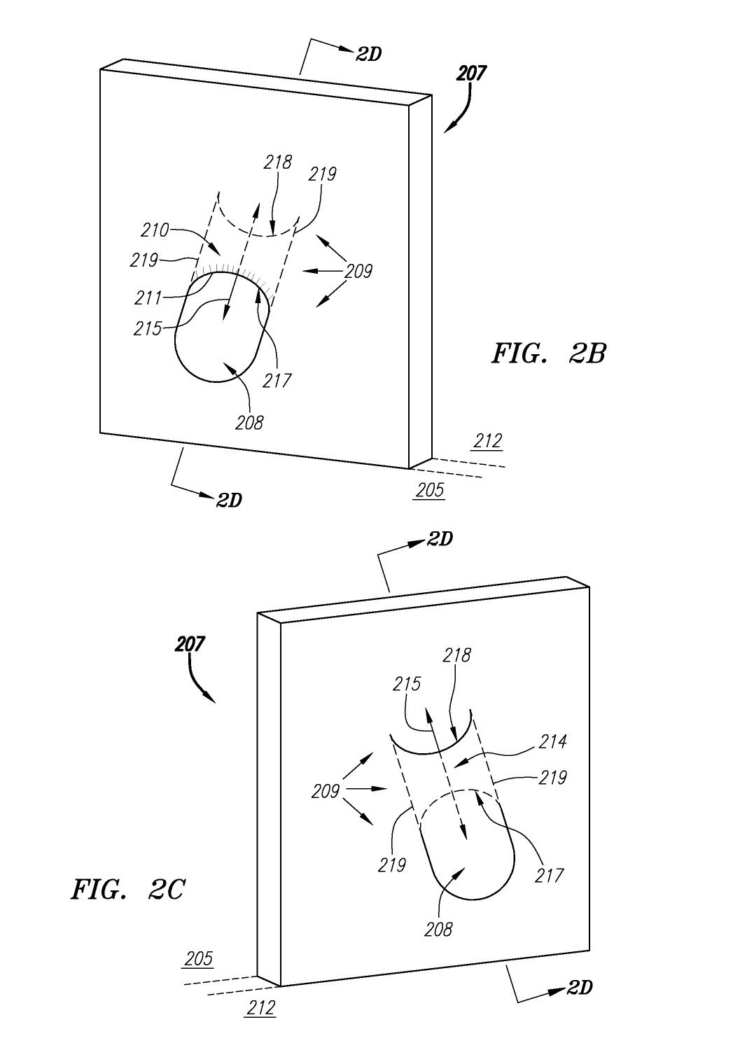

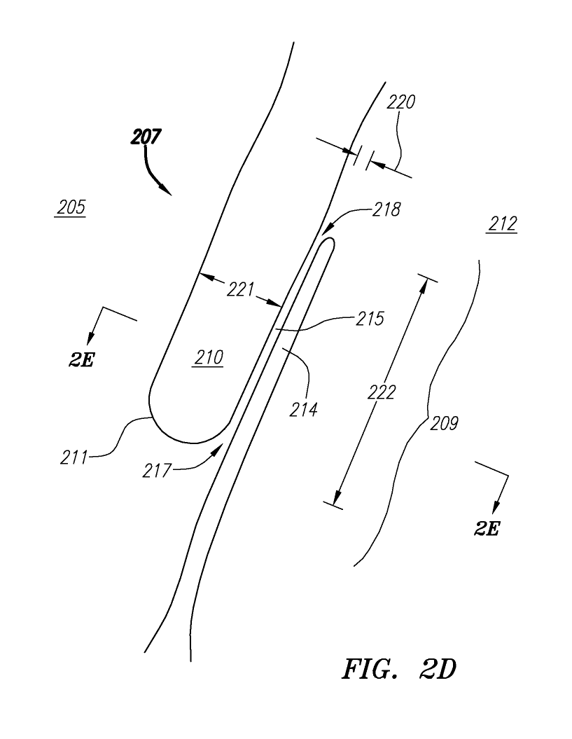

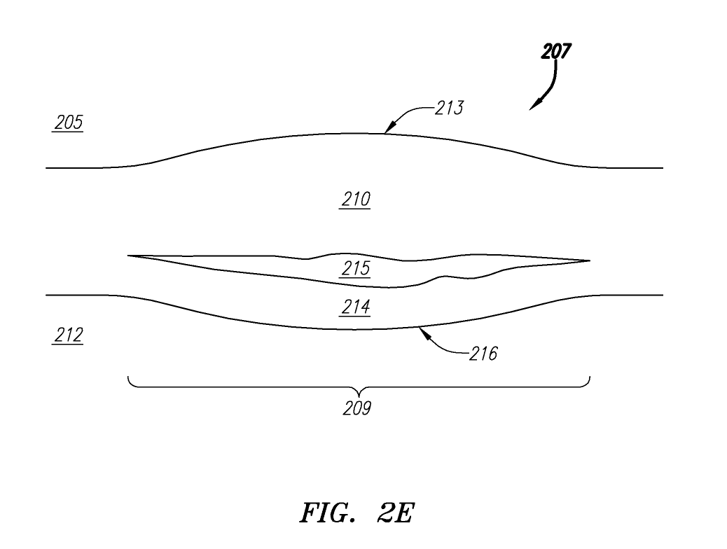

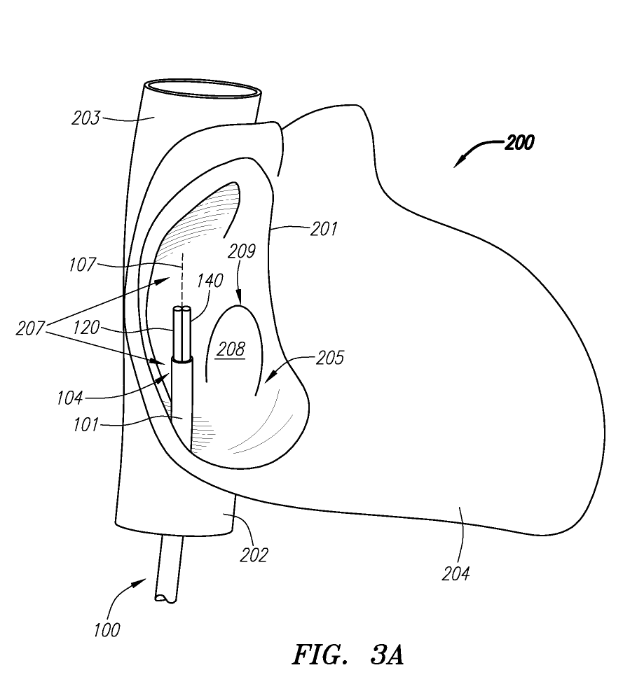

To better understand the many alternative embodiments of treatment system 100, the anatomical structure of an example human heart having a PFO will be described in brief. FIG. 2A is an exterior/interior view depicting an example human heart 200 with a portion of the inferior vena cava 202 and the superior vena cava 203 connected thereto. Outer tissue surface 204 of heart 200 is shown along with the interior of right atrium 205 via cutaway portion 201. Depicted within right atrium 205 is septal wall 207, which is placed between right atrium 205 and the left atrium located on the opposite side (not shown). Also depicted is fossa ovalis 208, which is a region of septal wall 207 having tissue that is relatively thinner than the surrounding tissue. PFO region 209 is located beyond the upper portion of the fossa ovalis 208.

FIG. 2B is an enlarged view of septal wall 207 depicting PFO region 209 in more detail as viewed from right atrium 205. PFO region 209 includes septum secundum 210, which is a first flap-like portion of septal wall 207. The edge of this flap above fossa ovalis 208 is referred to as the limbus 211. FIG. 2C is also an enlarged view of septal wall 207, instead depicting septal wall 207 as viewed from left atrium 212. Here, PFO region 209 is seen to include septum primum 214, which is a second flap-like portion of septal wall 207. Septum primum 214 and septum secundum 210 partially overlap each other and define a tunnel-like opening 215 between sidewalls 219 (indicated as dashed lines in FIGS. 2B-C, along with the outlines of fossa ovalis 208 and limbus 211) that can allow blood to shunt between right atrium 205 and left atrium 212. Tunnel 215 is commonly referred to as a PFO.

FIG. 2D is a cross-sectional view depicting an example PFO region 209 taken along line 2D-2D of FIGS. 2B-C. Here, it can be seen that septum secundum 210 is thicker than septum primum 214. Typically, the blood pressure within left atrium 212 is higher than that within right atrium 205 and tunnel 215 remains sealed. However, under some circumstances conditions can occur when the blood pressure within right atrium 205 becomes higher than the blood pressure within left atrium 212 and blood shunts from right atrium 205 to left atrium 212 (e.g., a valsava condition). Because most typical shunts occur in this manner and for purposes of facilitating the discussion herein, region 217 in FIG. 2D will be referred to as PFO entrance 217, and region 218 will be referred to as PFO exit 218.

FIG. 2E is a cross-sectional view depicting an example PFO region 209 taken along line 2E-2E of FIG. 2D. FIG. 2E depicts septum secundum 210 and septum primum 214 from a different perspective, again illustrating the structure of secundum 210 and primum 214 as flaps or deviated layers of septal wall 207. Reference numerals 213 and 216 indicate septal wall surfaces located in right atrium 205 and left atrium 212, respectively.

Many different variations of PFO's can occur. For instance, referring back to FIG. 2D, thickness 220 of septum primum 214, thickness 221 of septum secundum 210, overlap distance 222 and the flexibility and distensibility of both septum primum 214 and septum secundum 210 can all vary. In FIGS. 2B-C, PFO entrance 217 and PFO exit 218 are depicted as being relatively the same size with the width of tunnel 215, or the distance between sidewalls 219, remaining relatively constant. However, in some cases PFO entrance 217 can be larger than PFO exit 218, resulting in an tunnel 215 that converges as blood passes through. Conversely, PFO entrance 217 can be smaller than PFO exit 218, resulting in an opening that diverges as blood passes through. Furthermore, multiple PFO exits 218 can be present, with one or more individual tunnels 215 therebetween. Also, in FIGS. 2B-E, both septum primum 214 and septum secundum 210 are depicted as relatively planar tissue flaps, but in some cases one or both of septum primum 214 and septum secundum 210 can have folded, non-planar, highly irregular shapes.

As will be described in more detail below, treatment of a PFO preferably includes inserting treatment system 100 into the vasculature of a subject and advancing body member 101 through the vasculature to inferior vena cava 202 (optionally via a guidewire), from which access to right atrium 205 can be obtained. Once properly positioned within right atrium 205, delivery device 104 can be used to deliver one or more sutures 103 (not shown) to PFO region 209, preferably by inserting each suture 103 through septal wall 207 in a position suitable to allow at least partial closure of tunnel 215.

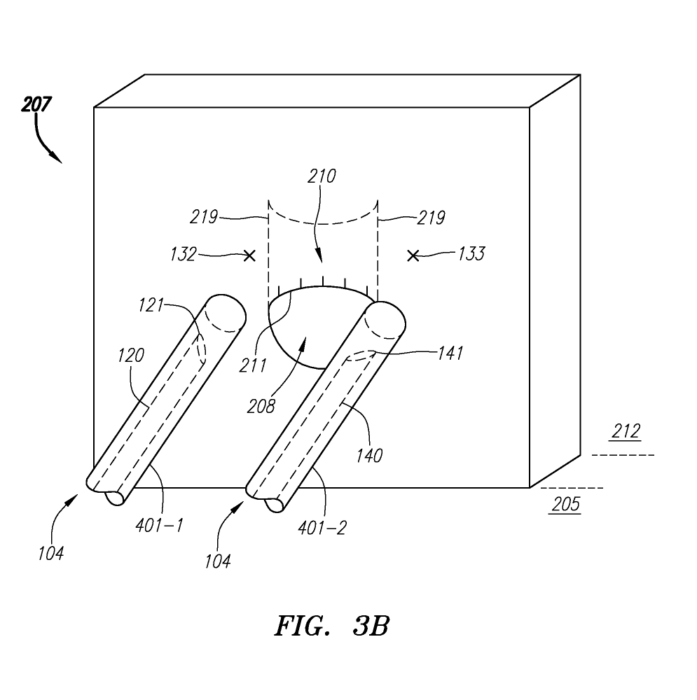

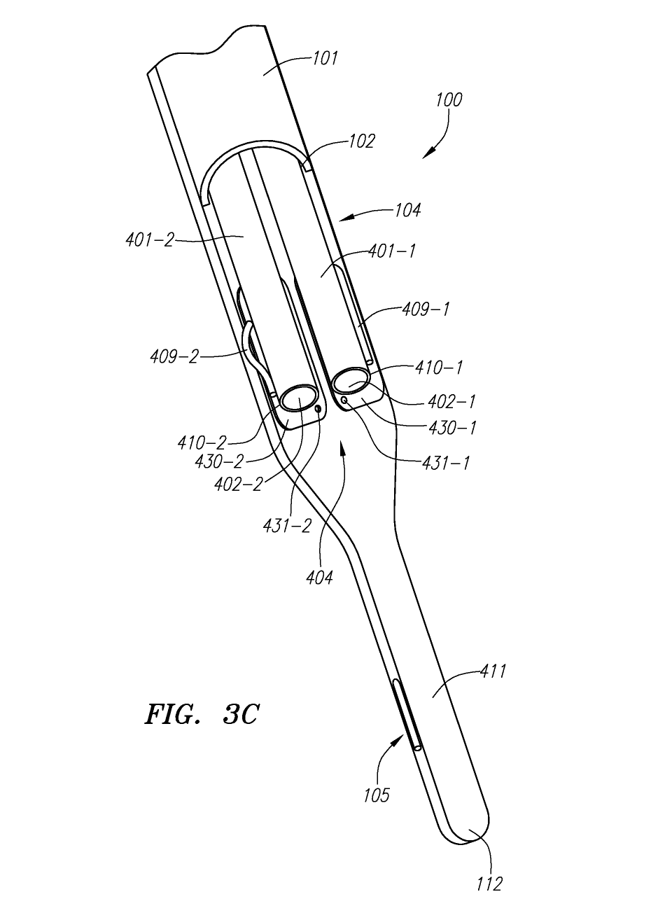

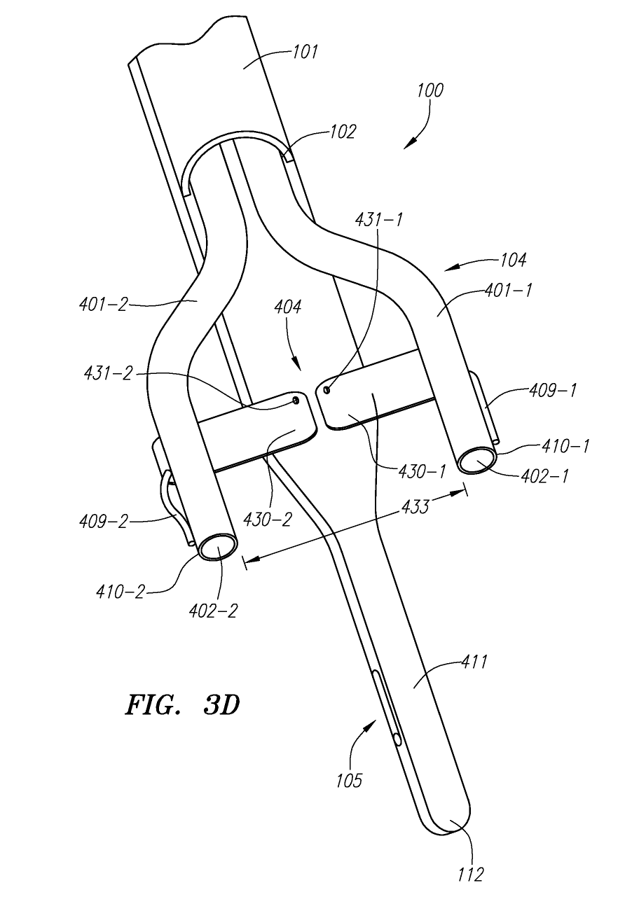

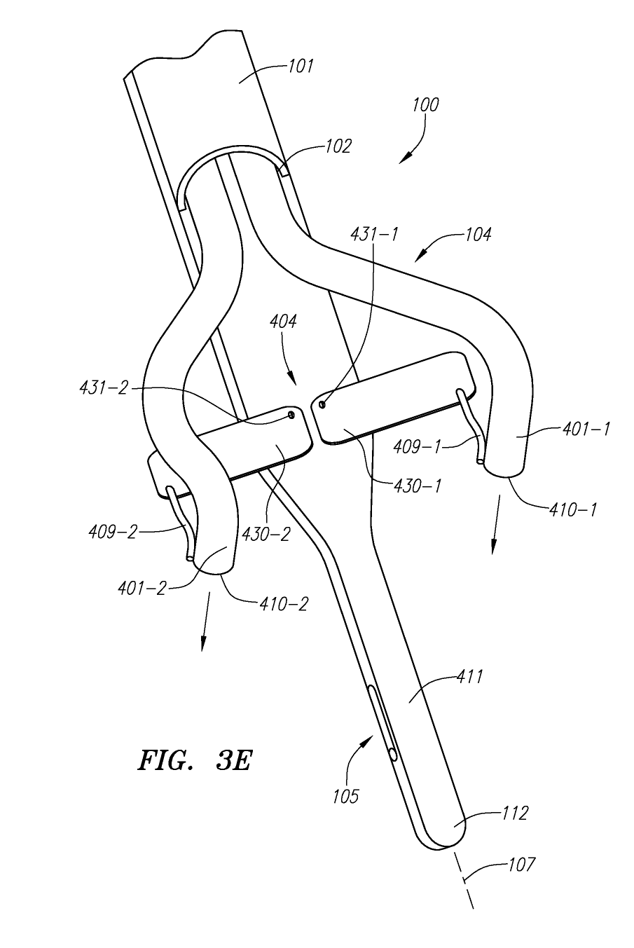

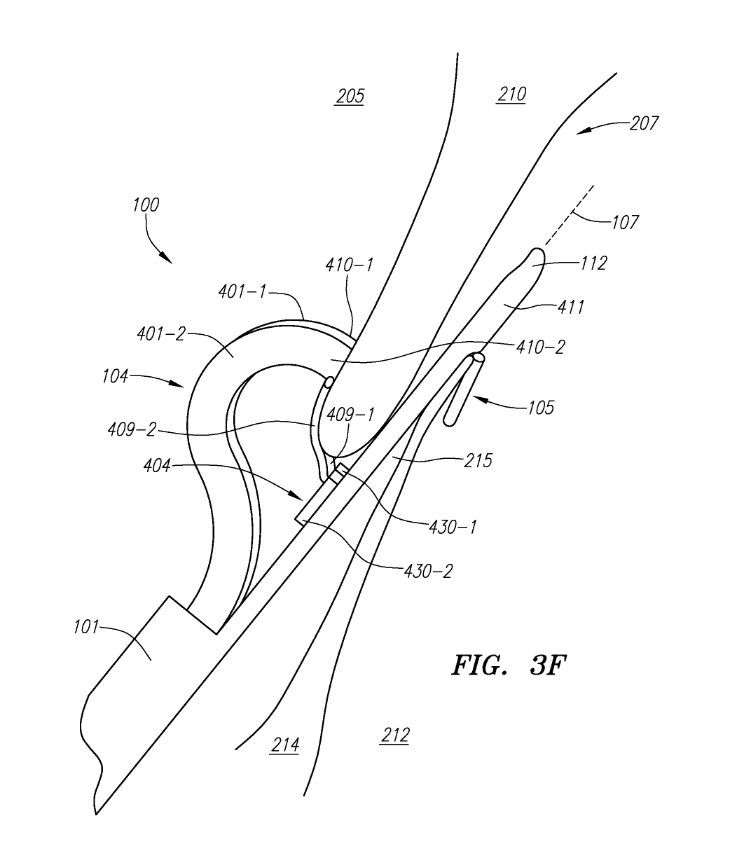

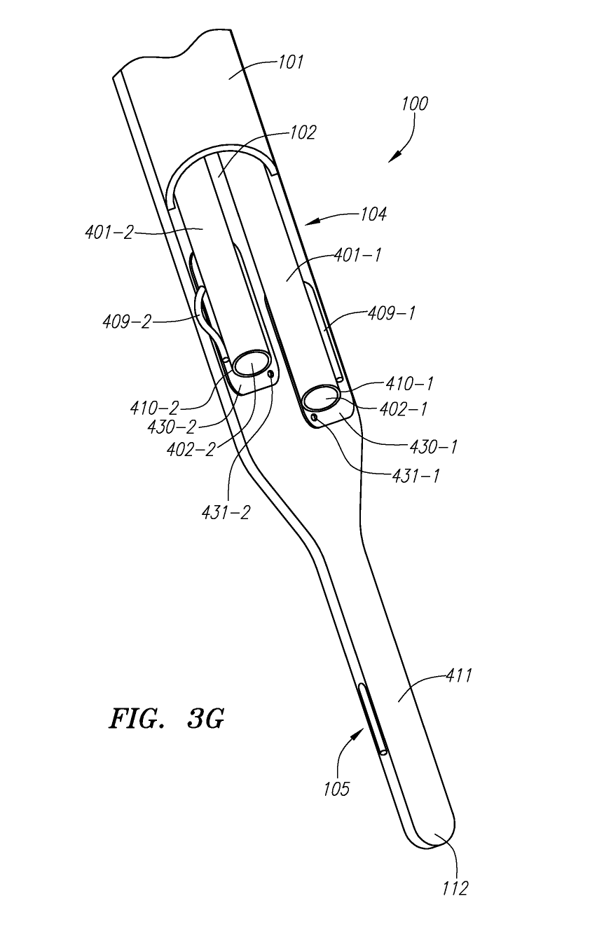

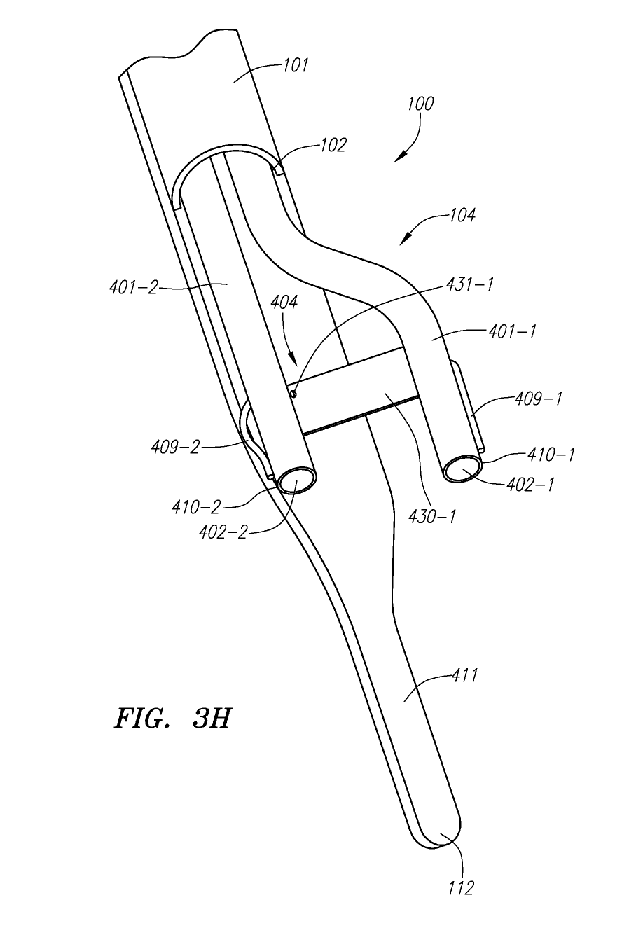

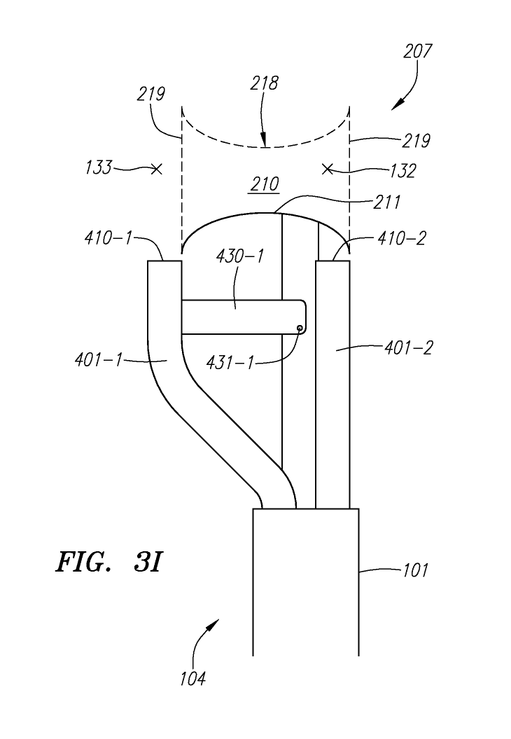

However, proper orientation of delivery device 104 with respect to septal wall 207 can be difficult due to the orientation of inferior vena cava 202 with respect to septal wall 207, as depicted in FIG. 3A. In the exemplary embodiments of system 100 described herein, proper orientation of needles 120 and 140 can be accomplished using an “off-axis” (OA) delivery configuration like that described in the incorporated co-pending U.S. patent application entitled “Systems and Methods for Treating Septal Defects” (Ser. No. 11/175,814). Although the off-axis delivery systems, devices and methods in this incorporated application are described primarily in the context of delivering coil-like implantable treatment devices, many of these systems, devices and methods can be used with the suture-based systems, devices and methods described herein. It should be noted that treatment of the PFO can be accomplished using any type of delivery system desired, not limited to the OA delivery systems described herein and those described within the incorporated '814 application. Furthermore, the OA delivery devices and methods described herein are not limited to use with the closure devices and methods described herein and can be used with other PFO and non-PFO treatment systems and methods.

FIG. 3B is a perspective view depicting an exemplary embodiment of delivery device 104 in proximity with septal wall 207, as viewed from right atrium 205. In this embodiment, delivery device 104 is preferably configured to use an OA configuration. Here, delivery device 104 preferably includes two tubular OA delivery members 401-1 and 401-2, each having a tubular elongate members 120 and 140 slidably housed therein, respectively. Members 120 and 140 are shown with dashed lines to indicate their location within OA delivery members 401-1 and 401-2. Also, the other components of delivery device 104 are not shown for clarity. Elongate members 120 and 140 are each needle-like with substantially sharp distal ends 121 and 141, respectively, for use in piercing, or penetrating, septal wall 207. Exemplary positions where members 120 and 140 can penetrate septal wall 207 are indicated with reference numerals 132 and 133, respectively. OA delivery members 401-1 and 401-2 are configured to adjust the orientation of elongate members 120 and 140, respectively, to facilitate penetration of septal wall 207. In this embodiment, elongate members 120 and 140 are configured to provide access to left atrium 212 to allow the closure of tunnel 215 with a suture 103. For ease of discussion herein, elongate members 120 and 140 will be referred to as needles 120 and 140, respectively, although it should be noted that members 120 and 140 are not limited to needles or needle-like members. Although not shown, suture 103 can be housed within needles 120 and/or 140.

FIG. 3C is a perspective view depicting an exemplary embodiment of treatment system 100 using an OA delivery configuration and including delivery device 104, stabilization device 105, and body member 101 having distal end 112. Here, delivery device 104 includes two OA delivery members 401-1 and 401-2, each of which are configured as an elongate flexible tubular member having an open distal end 410-1 and 410-2, respectively. Inner lumen 102 of body member 101 is preferably configured to slidably receive OA delivery members 401-1 and 401-2, such that OA delivery members 401-1 and 401-2 can be advanced both proximally and distally. OA delivery members 401-1 and 401-2 each have an inner lumen 402-1 and 402-2 that is preferably configured to slidably receive needles 120 and 140, respectively (not shown). Preferably, OA member distal ends 410-1 and 410-2 are coupled with an elongate support structure 411 of body member 101 via orientation device 404. Orientation device 404 is preferably used to place OA member distal ends 410-1 and 410-2 in the desired orientation with respect to septal wall 207. Here, the desired orientation is such that distal ends 410-1 and 410-2 face septal wall 207.

In this embodiment, orientation device 404 includes two pivot members 430-1 and 430-2, each having a first end coupled with elongate support structure 411 via hinges 431-1 and 431-2, respectively. Hinges 431 can be configured in any manner desired. Here, hinges 431-1 and 431-2 are shown in a pin/hole configuration. The opposite end of each pivot member 430-1 and 430-2 is flexibly coupled with arm members 409-1 and 409-2, respectively, which are in turn flexibly coupled with OA delivery members 401-1 and 401-2. Advancement of OA delivery members 401-1 and 401-2 in a distal direction causes the distal ends 410-1 and 410-2 to swing about hinges 431-1 and 431-2 into the configuration depicted in FIG. 3D. This separates distal ends 410-1 and 410-2 by a distance 433 and places OA delivery members 401-1 and 401-2 in the proper spaced relation for delivery of suture 103.

Further distal advancement of OA delivery members 401-1 and 401-2 causes arm members 409-1 and 409-2 to swing up and over pivot members 430-1 and 430-2, placing members 401-1 and 401-2 in the orientation depicted in FIG. 3E. Here, members 401-1 and 401-2 are oriented in the “off-axis” position, i.e., member distal ends 410-1 and 410-2 are axially oriented in a position off that of longitudinal axis 107 of body member 101. From this off-axis position, needles 120 and 140 can be advanced from within members 401-1 and 401-2 through septal wall 207, and suture 103 can be deployed, captured and retrieved in any manner desired, including, but not limited to those embodiments described below.

FIG. 3F is a partial cross-sectional view depicting this exemplary embodiment of system 100 in position within a septal wall 207. Here, body member 101 has been advanced through PFO tunnel 215 (preferably with the aid of a guidewire) and stabilization device 105 has been deployed over septum primum 214. OA delivery members 401-1 and 401-2 have been advanced into the off-axis position such that distal ends 410-1 and 410-2 are in contact with septum secundum 210. From this position, needles 120 and 140 can be deployed into the septal wall tissue.

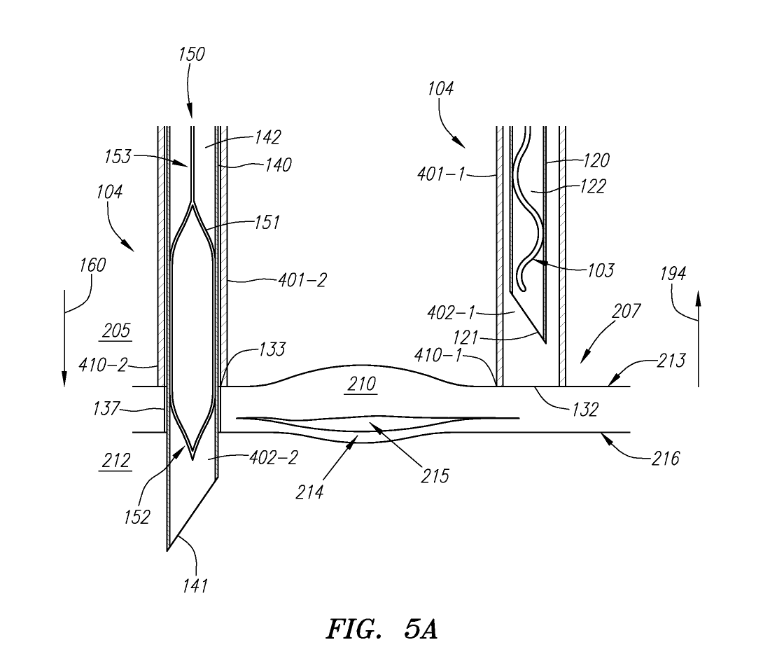

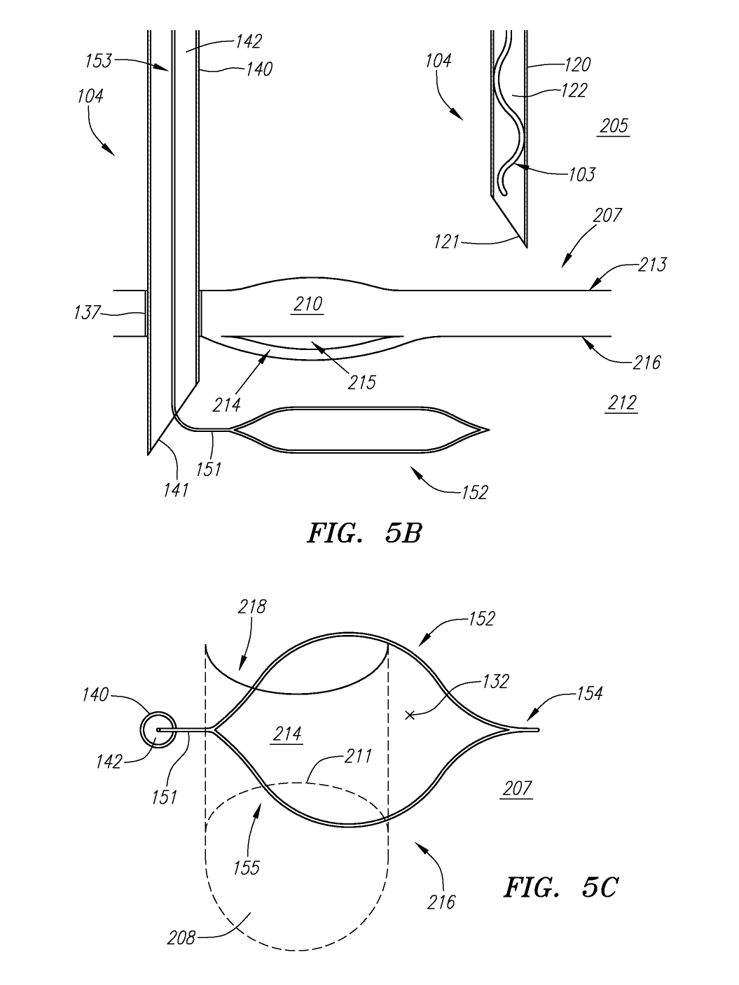

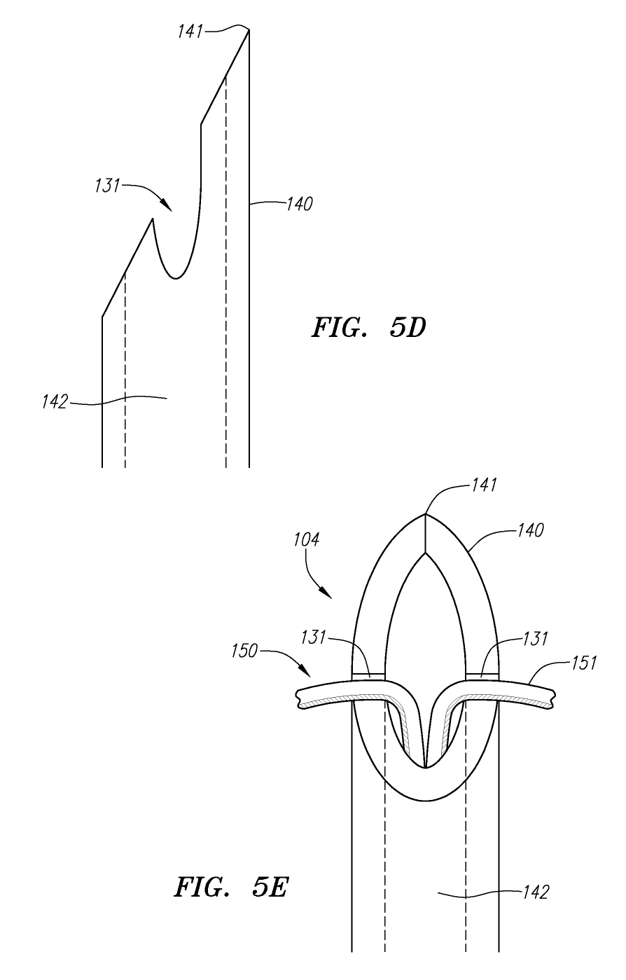

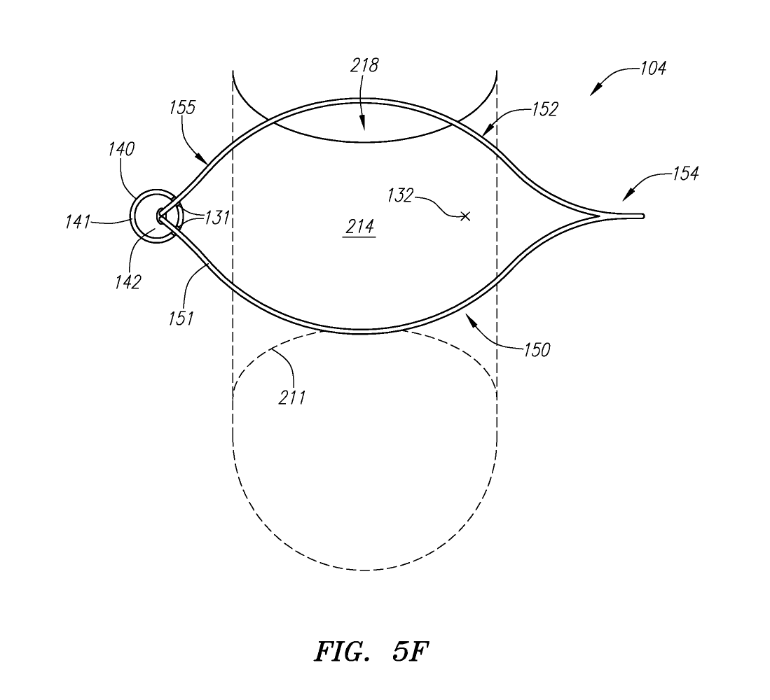

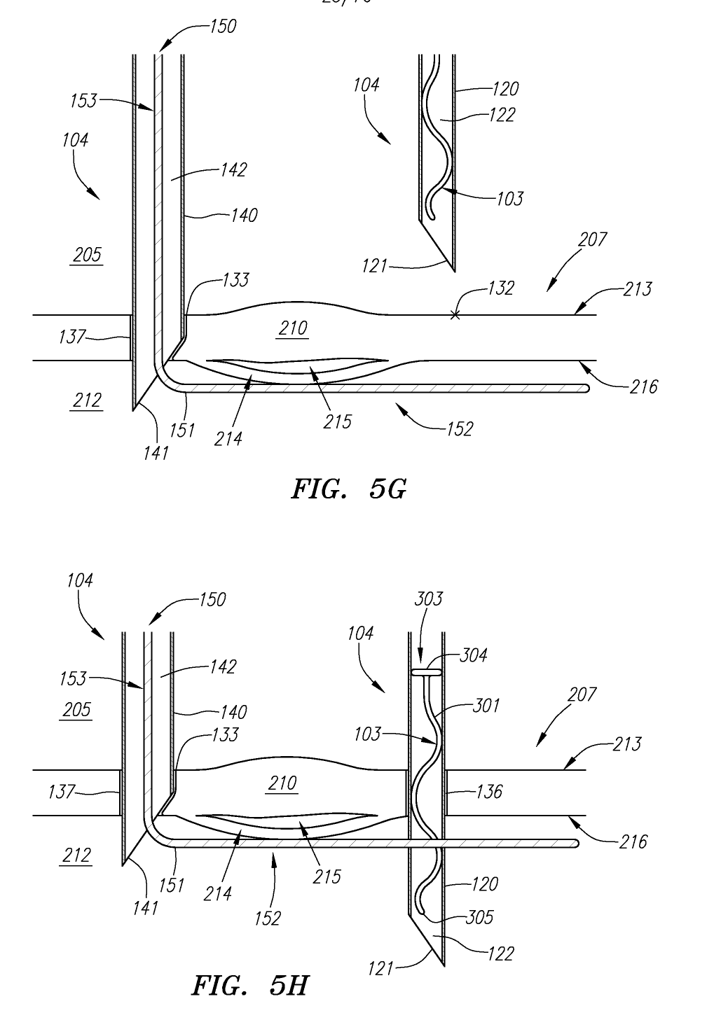

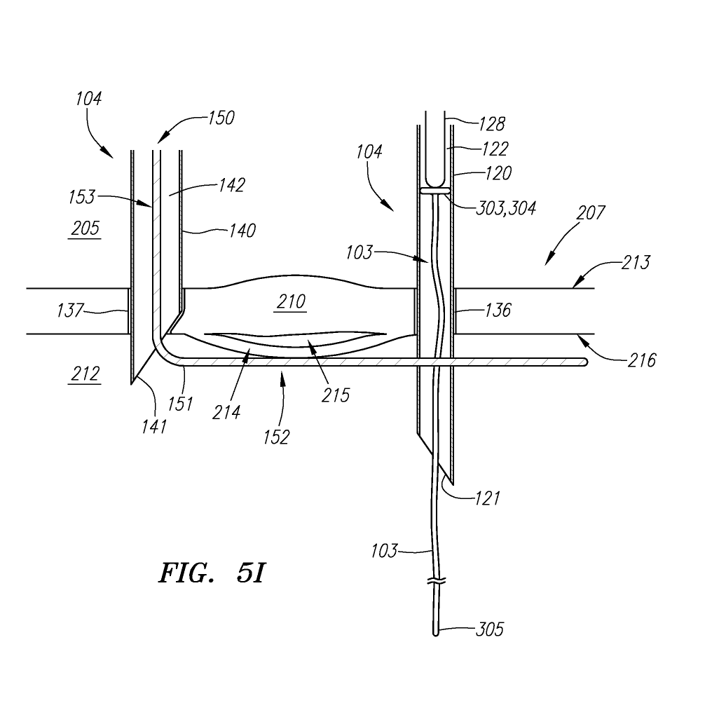

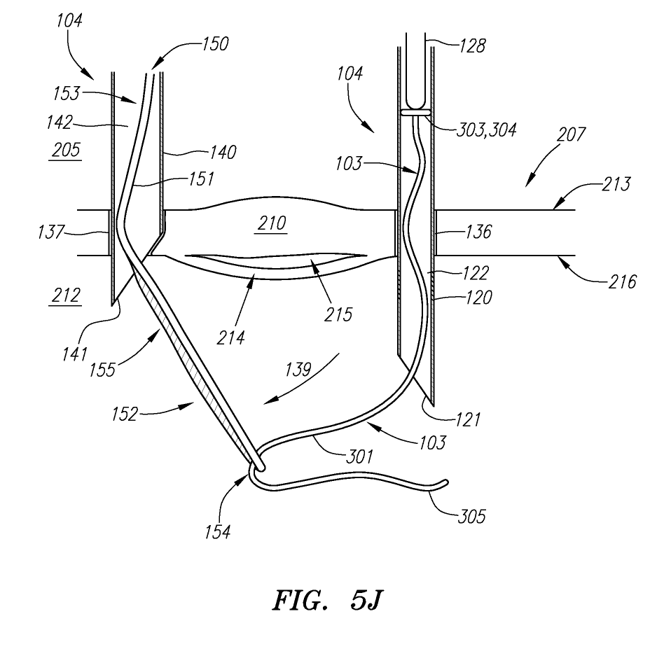

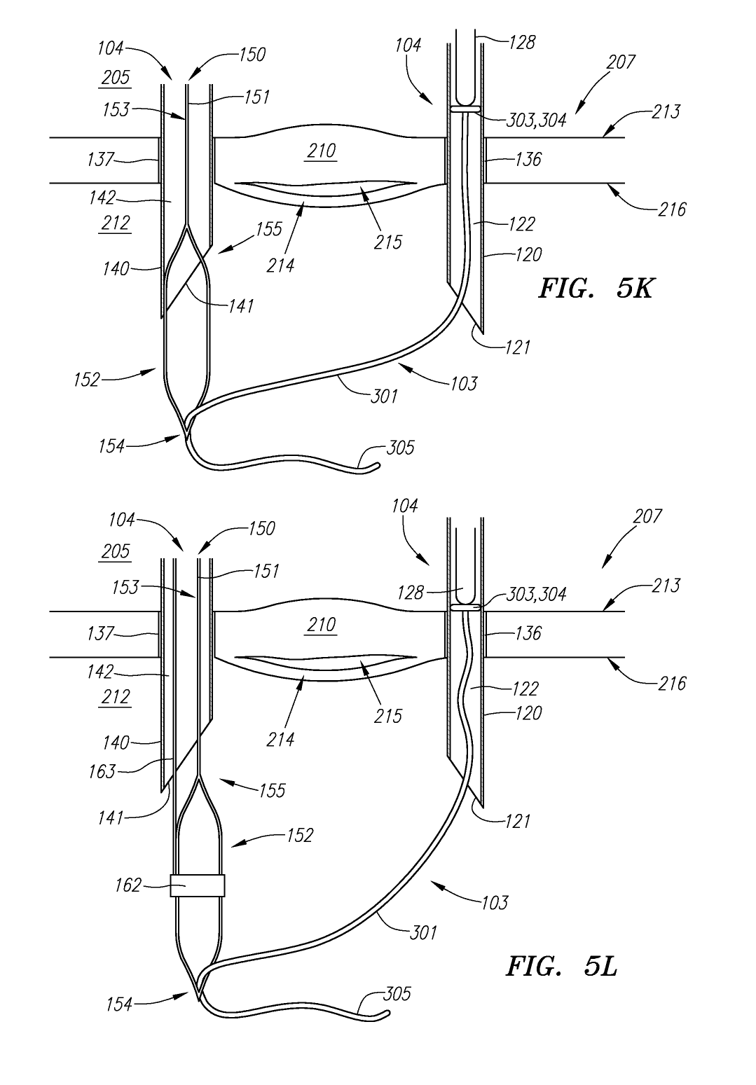

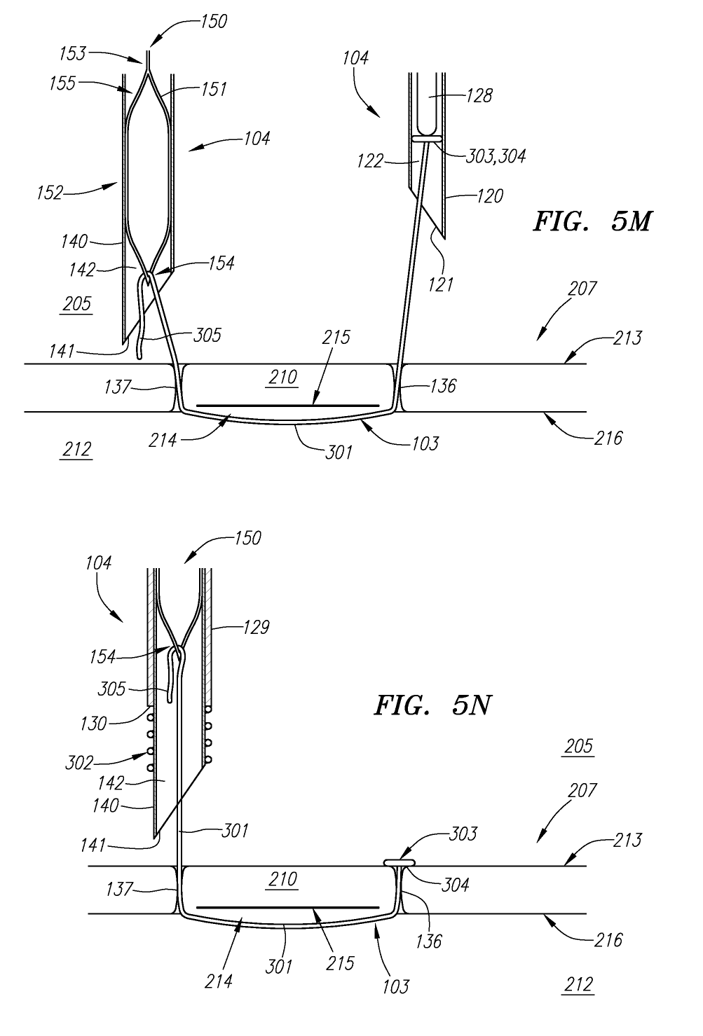

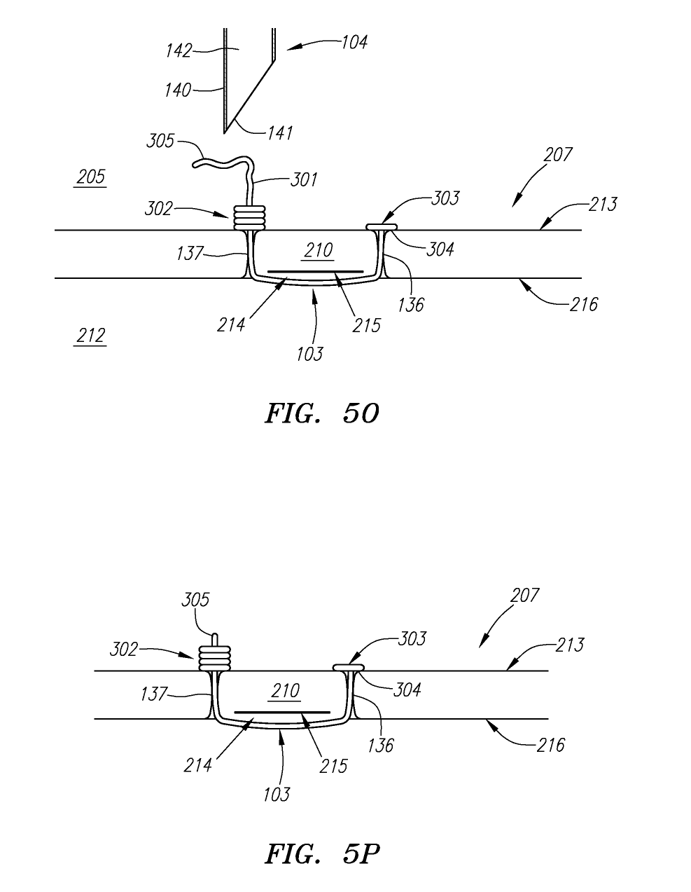

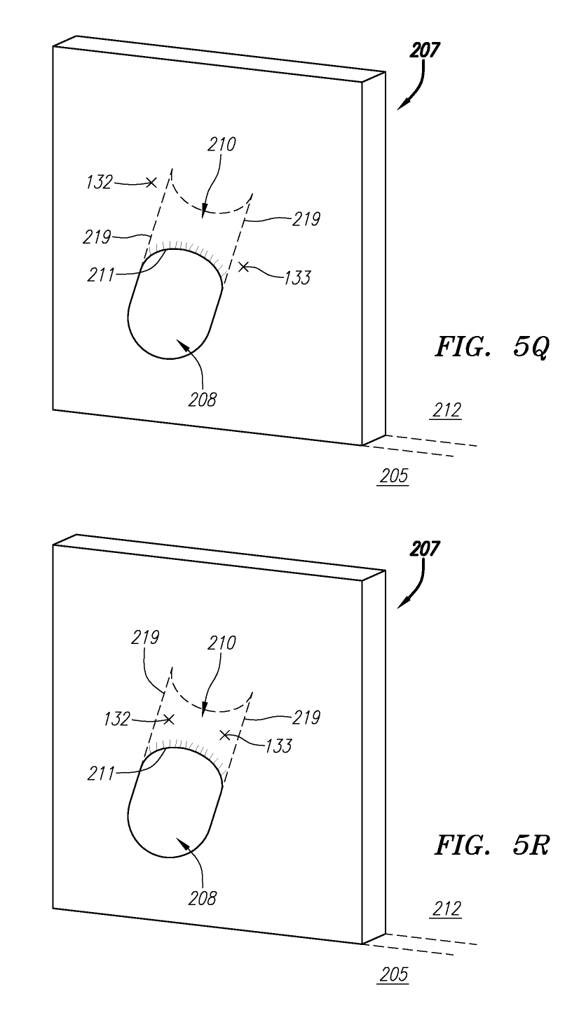

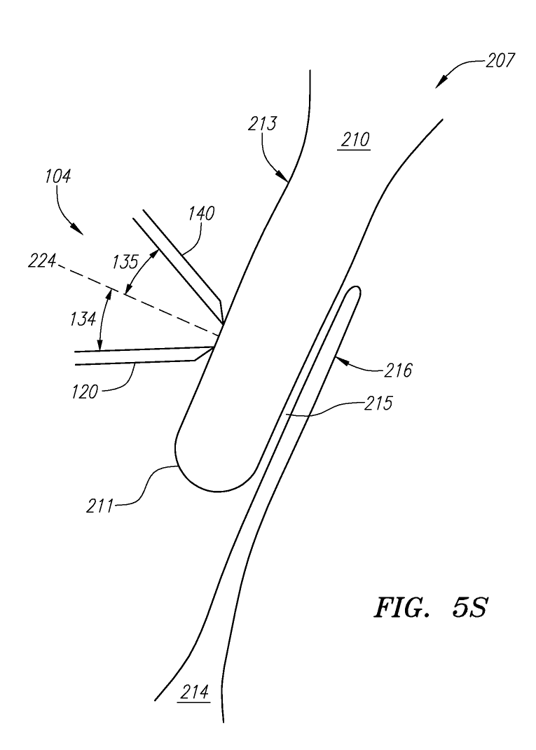

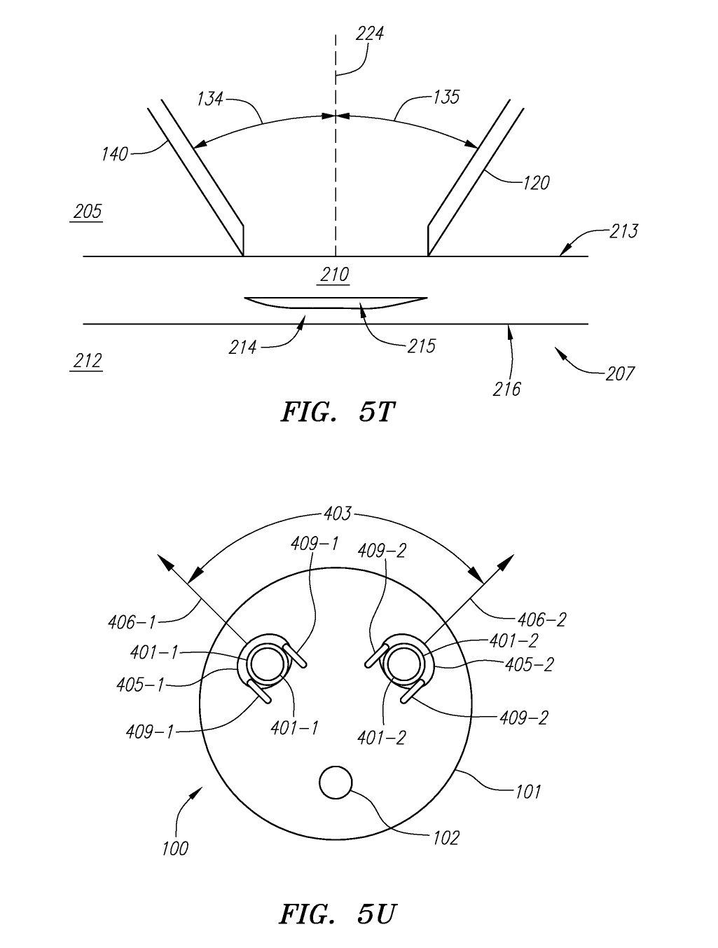

FIG. 3G depicts another exemplary embodiment of system 100. Here, OA delivery members 401-1 and 401-2 are located in staggered positions on body member 101. Specifically, member 401-1 is located distal to member 401-2. This staggered configuration can allow members 401-1 and 401-2 to be positioned in a manner that allows needles 120 and 140 to pierce septal wall 207 in “diagonally” oriented locations 132 and 133 such as those described with respect to FIG. 5Q below. In another exemplary embodiment, instead of staggering the positions where pivot members 430-1 and 430-2 are coupled with body member 101 as shown in FIG. 3G, arm member 409-2 can be made longer than arm member 409-1 (or vice-versa) to allow the penetration of septal wall 207 in diagonally oriented locations.

It should be noted that numerous techniques can be employed to achieve the off-axis orientation and system 100 is not limited to just the embodiments described here. For instance, in another exemplary embodiment, orientation device 404 can be omitted altogether and OA members 401-1 and 401-2 can be steerable via one or more pull-wires, similar to that described in the incorporated co-pending application entitled “Systems and Methods for Treating Septal Defects” (Ser. No. 11/175,814).

It should also be noted that delivery device 104 can also be configured such that only one of OA delivery members 401-1 and 401-2 pivots outward, while the other remains stationary with respect to body member 101. FIG. 3H is a perspective view depicting an exemplary embodiment where OA member 401-1 is pivoted outwards and OA member 401-2 remains stationary. This embodiment can be used in an exemplary procedure where body member 101 is positioned relatively closer to one sidewall 219 of tunnel 215, as depicted in FIG. 3I, which is an end on view of septal wall 207 taken from right atrium 205, showing device 104 with.

Here, elongate support structure 411 has been advanced partially into tunnel 215 and OA member 401-2 is located adjacent one sidewall 219. OA member 401-1 has been pivoted outwards to increase the coverage area over tunnel 215. The intended penetration sites for OA members 401-1 and 401-2 are referenced as points 132 and 133, respectively. Delivery device 104 can be configured such that the device is predisposed towards one side of tunnel 215, such as by configuring body member 101 with a curved portion (not shown), in which case only one of OA delivery members 401-1 and 401-2 can be made to pivot (although both can be configured to pivot, regardless of whether they are actually pivoted during the closure procedure).

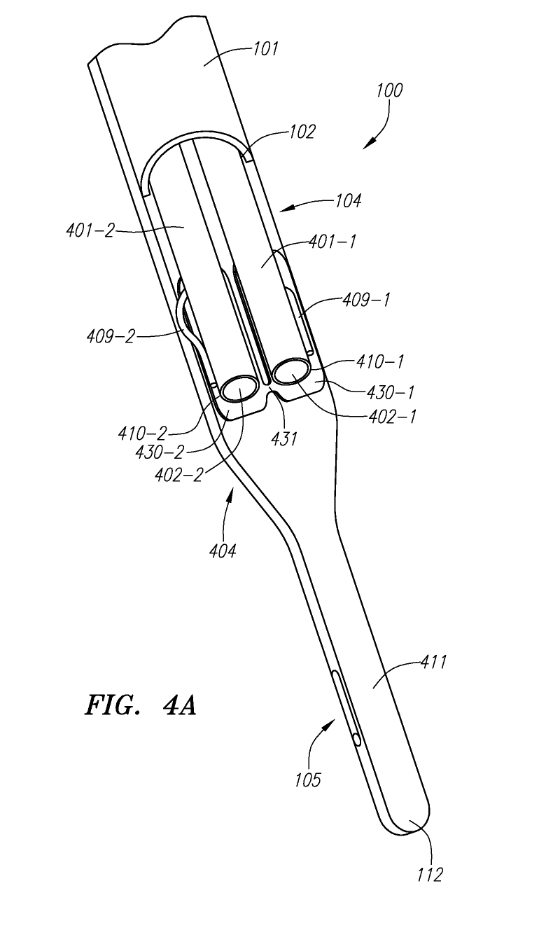

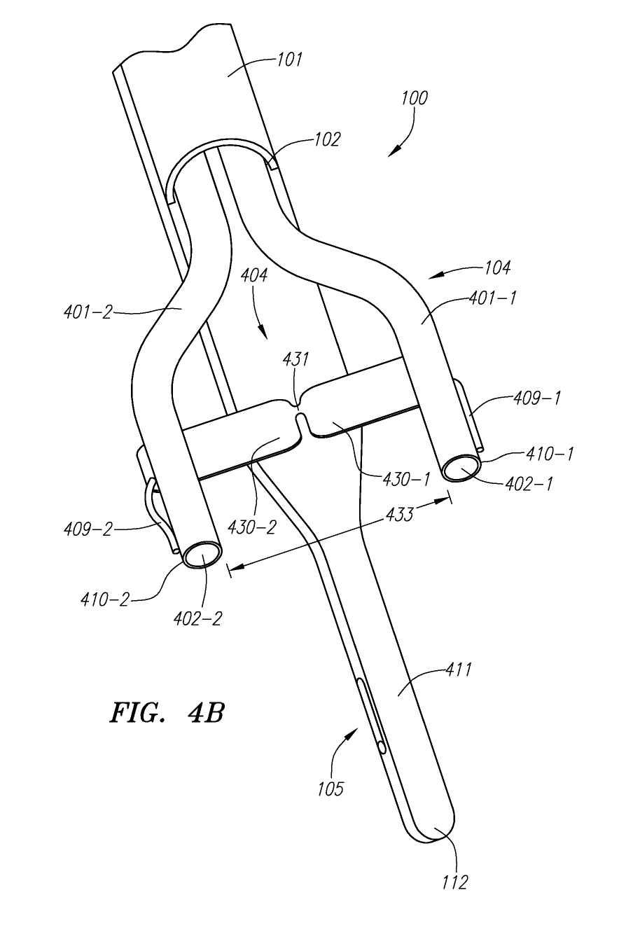

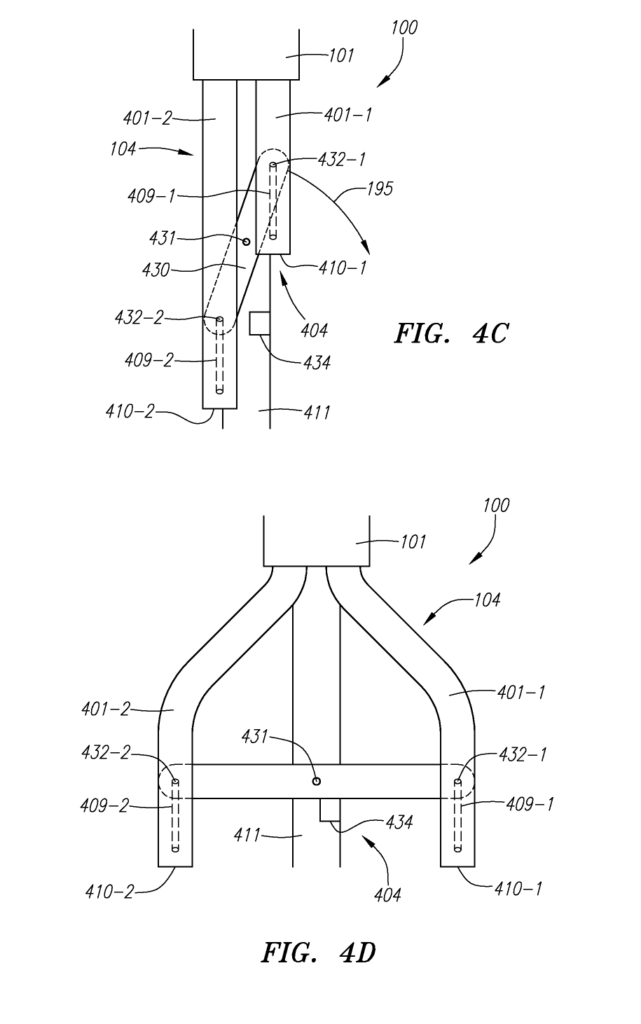

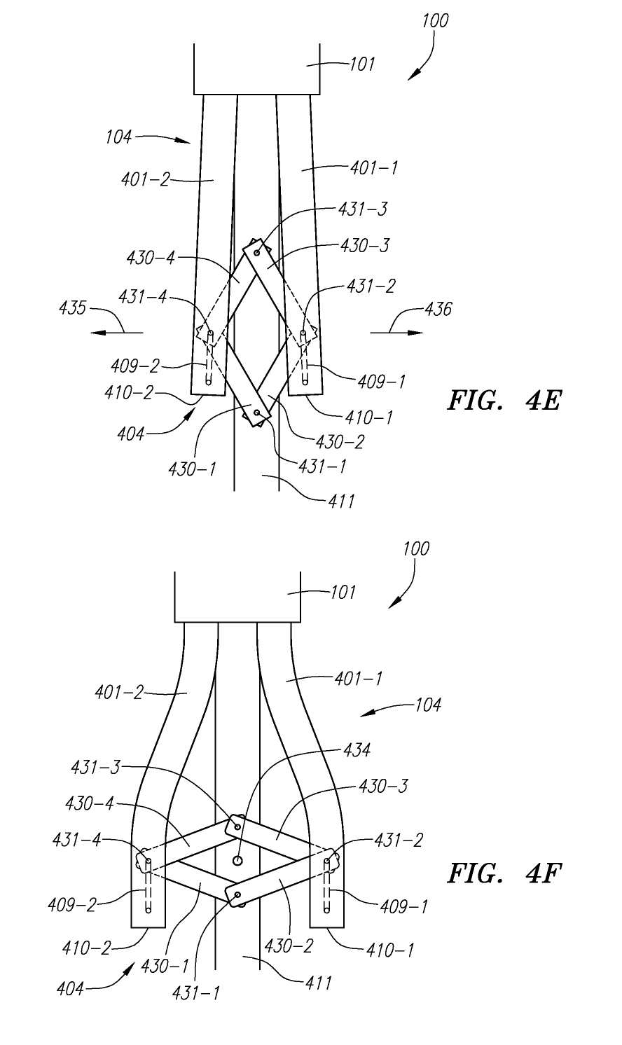

FIGS. 4A-B are perspective views depicting an exemplary embodiment where orientation device 404 is a monolithic flexible structure with hinge 431 configured as a “living hinge,” i.e., a relatively thin bendable section that can withstand repeated motion without breakage. FIG. 4A depicts OA members 401-1 and 401-2 prior to advancement. Hinge 431 is preferably coupled to support section 411 such that pivot members 430-1 and 430-2 are free to rotate or swing about hinge 409, as depicted in FIG. 4B. Hinge 431 can be fabricated out of any material desired that allows for the repeated motion. In one exemplary embodiment hinge 431 is fabricated from an elastomeric polymer.

FIG. 4C is a top down view depicting another exemplary embodiment of system 100 where orientation device 404 includes a single, preferably rigid, bar-like pivot member 430 coupled with support section 411 via a centrally-located hinge 431. Each end of pivot member 430 is coupled with arm members 409-1 and 409-2 via hinges 432-1 and 432-2, respectively (pivot member 430, arm members 409-1 and 409-2 and hinges 432-1 and 432-2 are each indicated with dashed lines due to their placement beneath OA members 401-1 and 401-2). For deployment, OA member 401-2 is advanced distally and OA member 401-1 is retracted proximally to cause rotation of pivot member 430 about hinge 431 in direction 195, into the position shown in FIG. 4D. An abutment 434 can be placed on support structure 411 to stop rotation at the intended position, if desired. From the position shown here in FIG. 4D, members 401-1 and 401-2 can be advanced distally to gain orientation in the off-axis position. In another exemplary embodiment, pivot member 430 can be left uncoupled with support section 411 such that it is “free-floating.”

FIG. 4E is a top down view depicting another exemplary embodiment of system 100 where orientation device 404 includes a multiple member linkage. Specifically, orientation device includes four pivot members 430-1, 430-2, 430-3 and 430-4. Each of pivot members 430-1 through 430-4 is flexibly coupled together with one of hinges 431-1, 431-2, 431-3 and 431-4. Here, pivot members 430-1 and 430-2 are coupled together via hinge 431-1, which is also coupled to elongate support structure 411. In this embodiment, the coupling is provided by routing the pin used to form hinge 431-1 into support structure 411 as well.

Pivot members 430-2 and 430-3 are coupled together via hinge 431-2, pivot members 430-3 and 430-4 are coupled together via hinge 431-3, and pivot members 430-4 and 430-1 are coupled together via hinge 431-4. Hinges 431-2 and 431-4 are also coupled to arm members 409-1 and 409-2, respectively. For deployment, OA members 401-1 and 401-2 are advanced distally from the position depicted in FIG. 4E to that depicted in FIG. 4F. Hinge 431-3 is preferably configured to move freely with respect to elongate support structure 411. Distal advancement of members 401-1 and 401-2 causes hinge 431-3 to move distally while hinges 431-4 and 431-2 move outwards in directions 435 and 436 (shown in FIG. 4E), respectively. (Although hinge 431-3 is not coupled with support structure 411 in this embodiment, hinge 431-3 can be configured to slide within a track in support structure 411 to provide additional support.) This four pivot member configuration allows members 401-1 and 401-2 to be moved distally and proximally together in a “lockstep” fashion until reaching abutment 434. However, orientation device 404 can also be configured with only two pivot members 430-1 and 430-2 if desired. From the position shown in FIG. 4F, members 401-1 and 401-2 can be advanced further distally to gain orientation in the off-axis position and deploy members 120 and 140.

In another exemplary embodiment, one or more hinges 431-1 through 431-4 can be biased towards a predetermined state, such as the state described with respect to FIG. 4E. Hinges 431-1 through 431-4 can be spring-like or elastic living hinges or any other configuration that exerts a bias. One of skill in the are will readily recognize the many possible configurations that can be used.

FIGS. 5A-S depict the use of one exemplary embodiment of treatment system 100 at various times during an exemplary PFO closure procedure. FIG. 5A is a partial cross-sectional view depicting an exemplary embodiment of system 100. Here, OA delivery members 401-1 and 401-2 have been advanced into contact with septal surface 213. Reference marks 132 and 133 indicate the locations where elongate members 120 and 140, respectively, are intended to pierce septal wall 207. Needle 140 has been advanced distally from distal end 410-2 of OA delivery member 401-2 and through septal wall 207. In this embodiment, needle 140 has been used to penetrate septal wall 207 in a location alongside of tunnel 215, although other locations across tunnel 215 can also be used. The opening created by advancement of needle 140 through septal wall 207 is referenced here via numeral 137. Also shown is needle 120 having inner lumen 122 with suture 103 located therein.

As can be seen here, an elongate, capture device 150 is housed within an inner lumen 142 of needle 140. Capture device 150 can be any device having a configuration adapted for snaring, capturing, engaging or otherwise obtaining some degree of control over another device, which can include closure device 103. In this and some other embodiments described herein, capture device 150 can be configured as a snare-like device, although capture device 150 is not limited to such. Snare-like device 150 can include a body 151 having a snare head portion 152 and a base portion 153 and, for ease of discussion, will be referred to herein simply as snare 150. Snare 150 can be in the form of a simple wire loop, or can have a more complex configuration such as those which will be described herein. Snare head portion 152 is preferably deformable between an open and a closed configuration for use in capturing suture 103. Snare head portion 152 preferably has a predisposed bias towards the open configuration. Snare 150 is shown here maintained in the closed configuration by the walls of needle 140. Upon advancement from within inner lumen 142, snare head portion 152 is free to expand into the open configuration as will be discussed below.

To allow deformability between the biased open configuration and the closed configuration, snare body 151 is preferably formed from a flexible elastic or superelastic material, such as NITINOL, stainless steel, elgiloy, polymeric materials and the like. It should be noted that snare 150 is not limited to structures that are deformable from one configuration to another. In other embodiments, snare 150 can be configured to mechanically switch between the open and closed configurations.

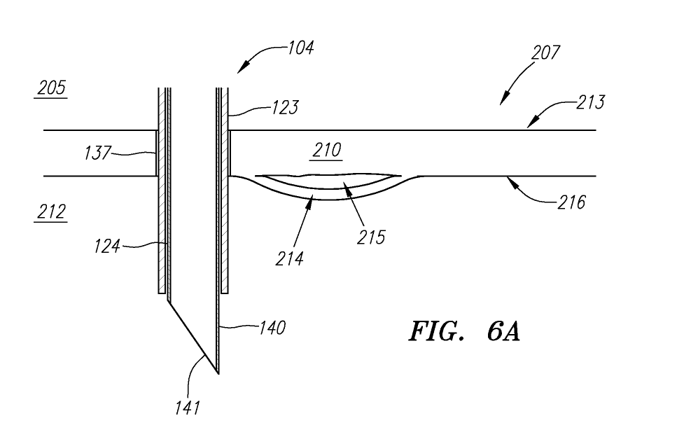

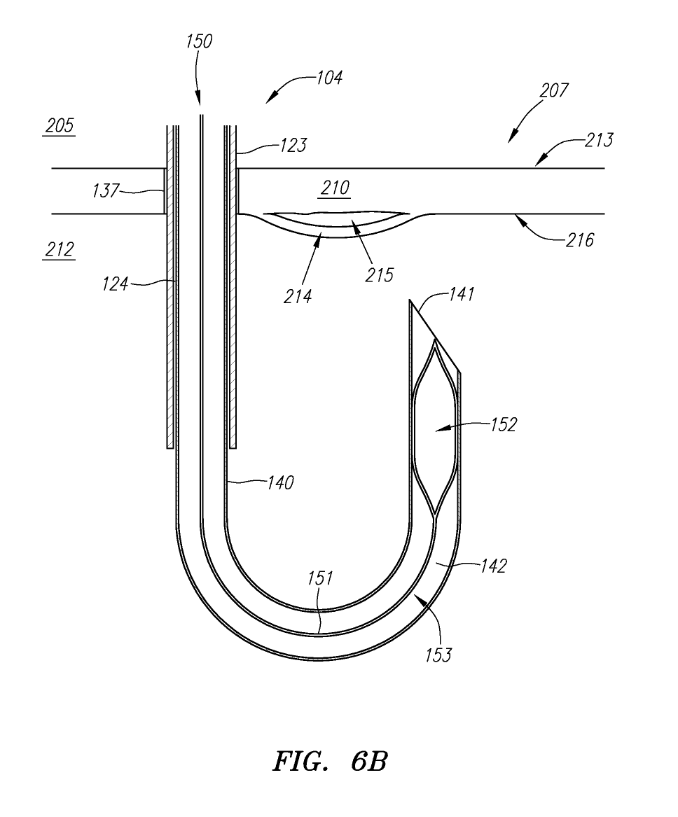

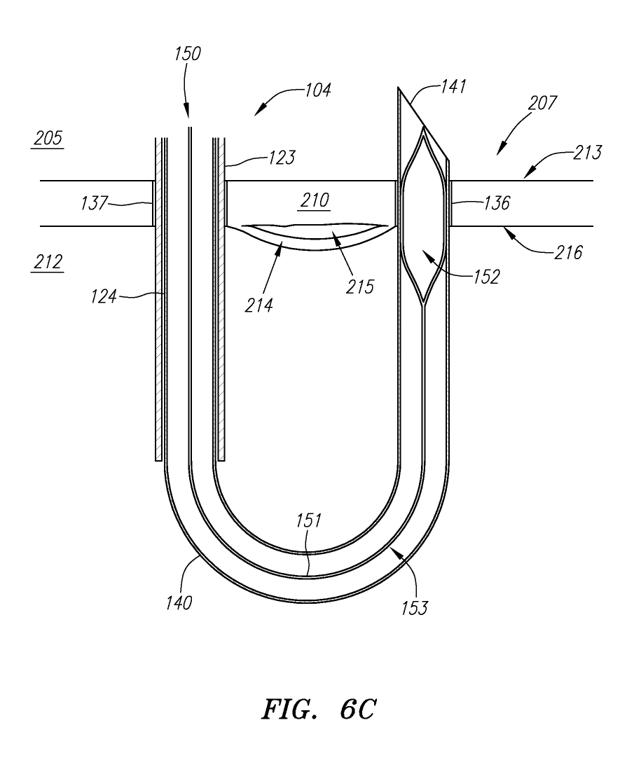

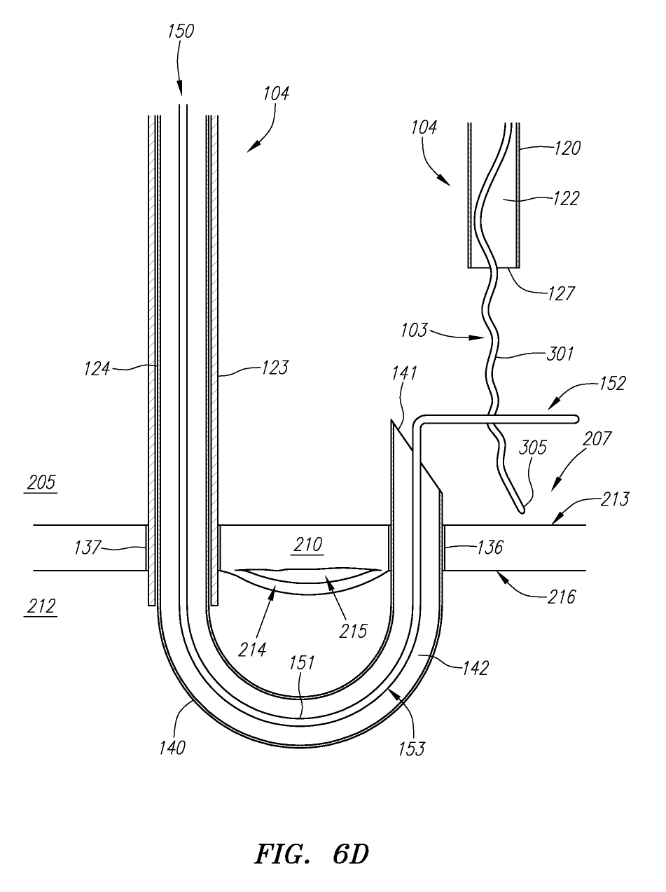

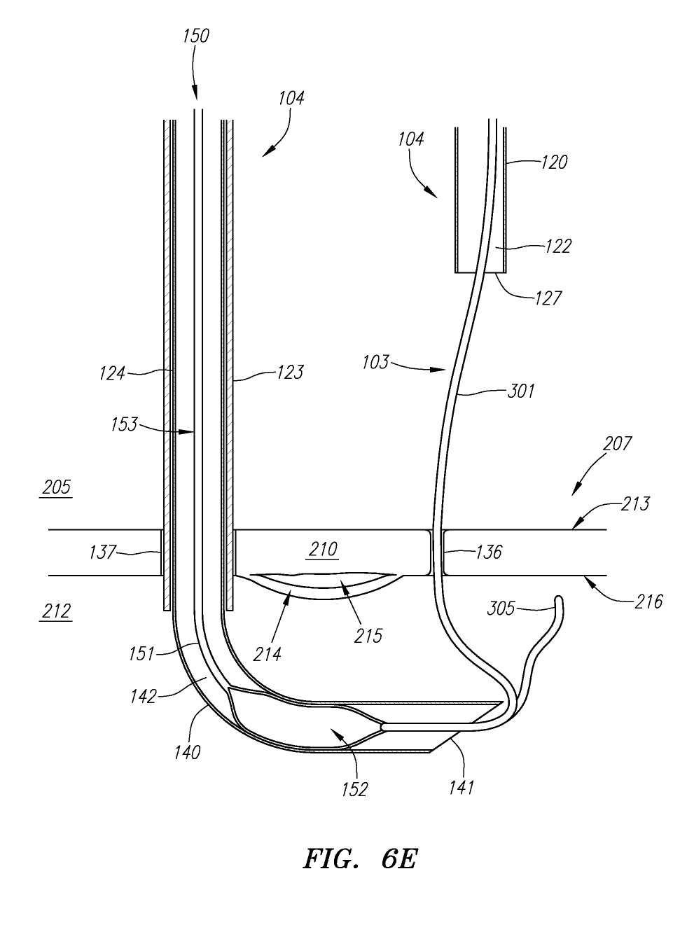

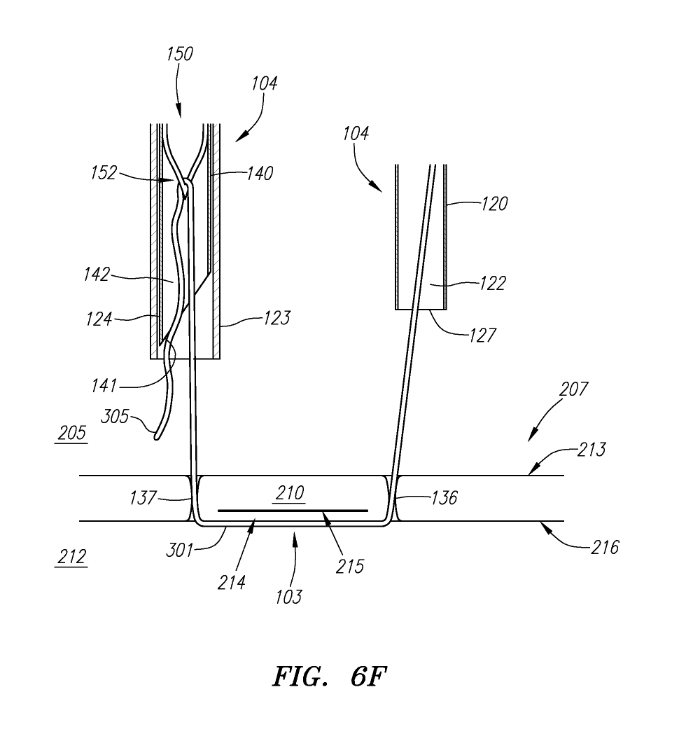

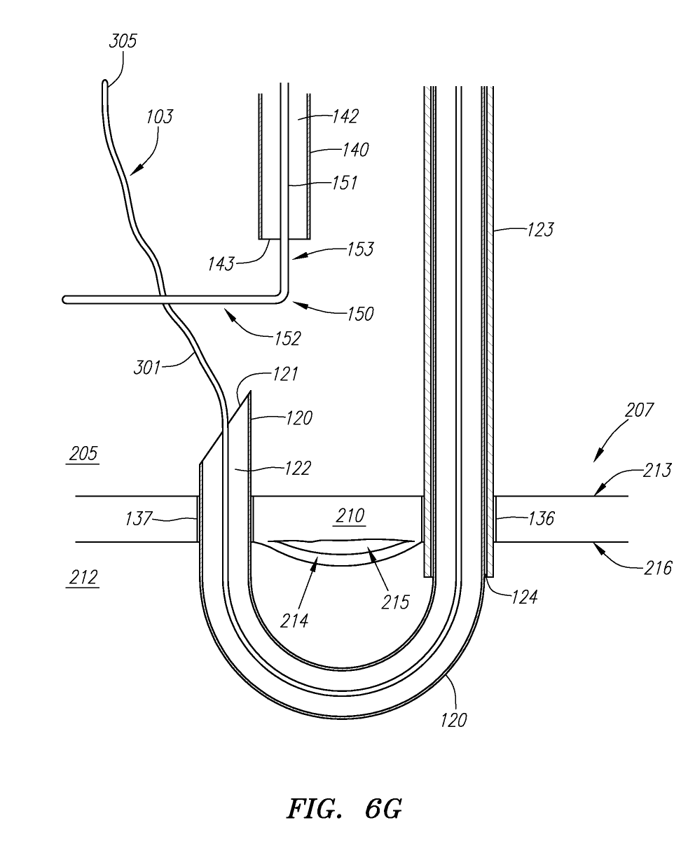

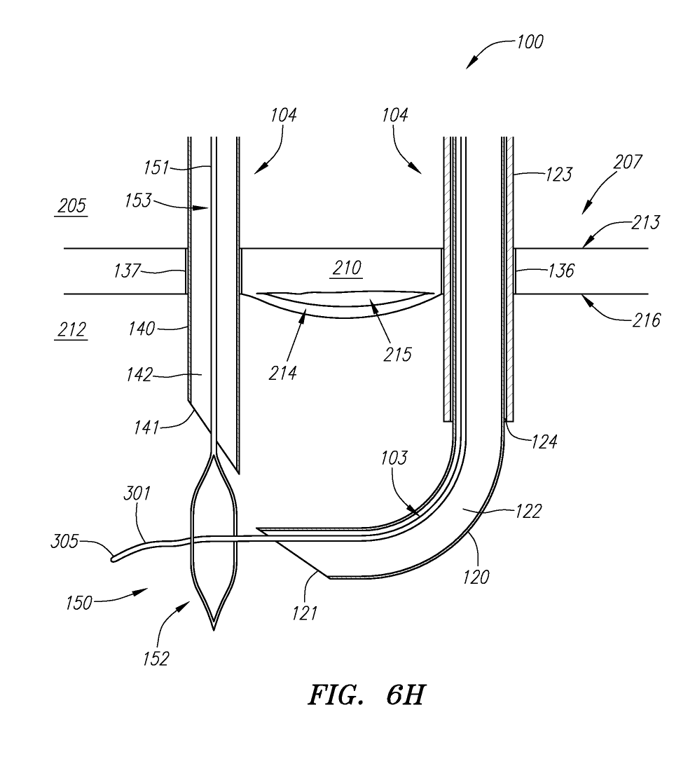

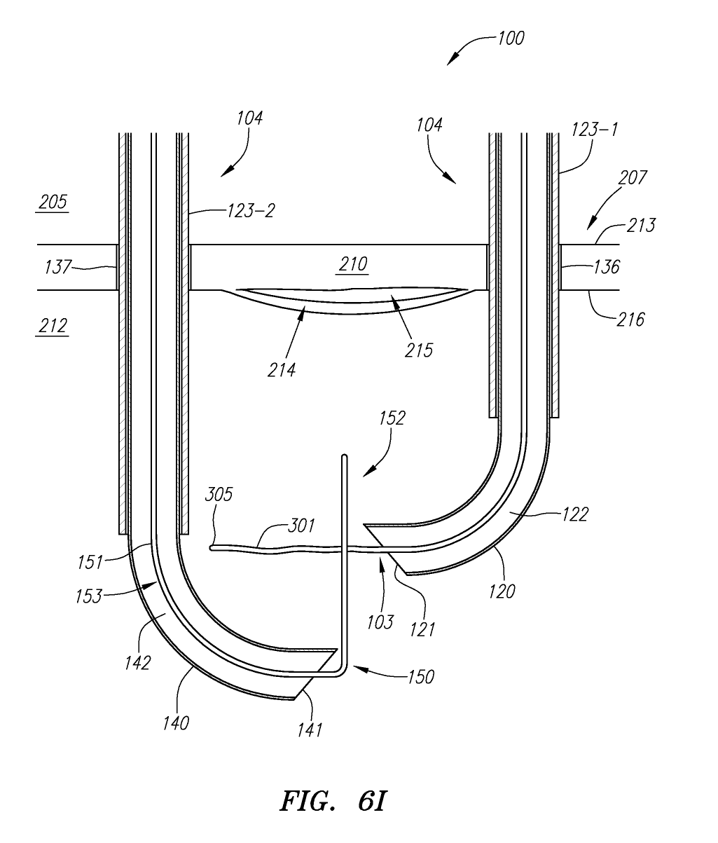

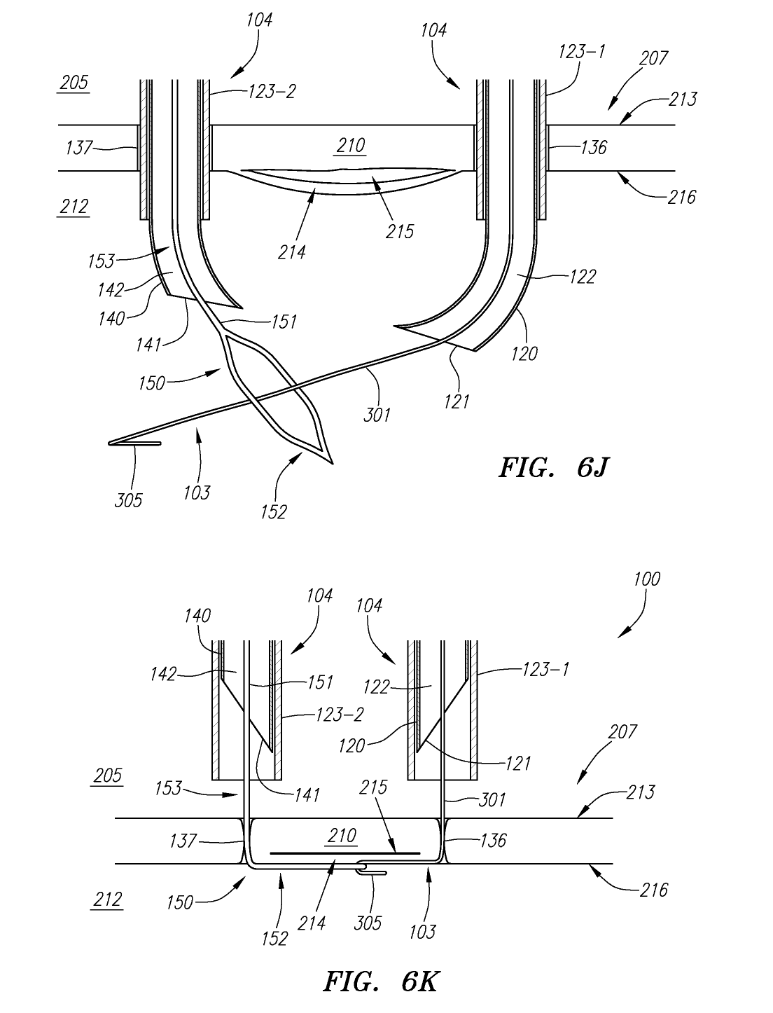

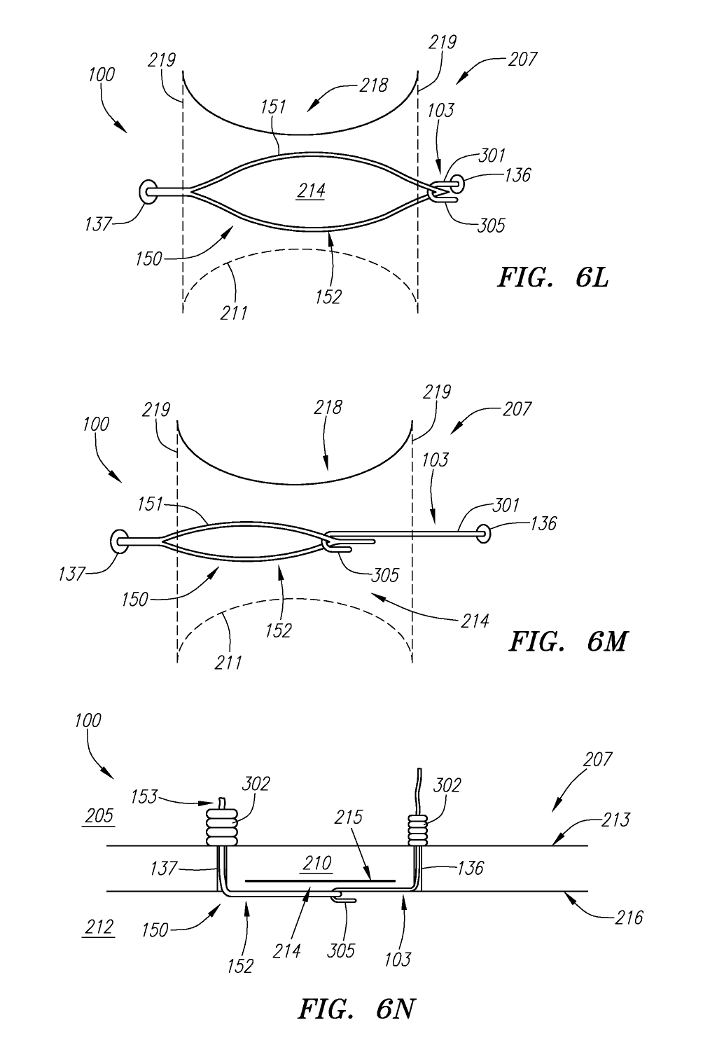

Throughout this description, reference will be made to movement in the “proximal” and “distal” directions. FIG. 5A depicts distal direction 160 as being generally away from the user and, in this case, progressing from right atrium 205 towards septal wall 207. Proximal direction 194 is depicted as being generally towards the user and, in this case, moving from septal wall 207 towards right atrium 205. It should be noted here that the orientation of distal direction 160 and proximal direction 194 are dependent on the path the user takes through heart 200. For instance, directions 160 and 170 would be reversed if the system 100 entered left atrium 212 first and proceeded towards right atrium 205. As will become apparent, certain components of system 100 can be placed in a curved state such that any movement of the component occurs in both the proximal and distal directions (e.g., snare 150 and member 140 as will be described with respect to the embodiment of FIG. 6B). In these instances, in order to maintain consistency, movement in the distal direction 160 and proximal direction 194 will be in reference to the portion of the component that does not enter the curved state.

Also for consistency, when referring to a portion of an object as being distal or proximal, this terminology, once applied, will be maintained regardless of how the orientation of the object is subsequently altered. For instance, as will be described with respect to FIG. 5H, suture 103 includes a proximal end 304 and a distal end 305. This terminology is applied based on the orientation of suture 103 within member 120. These ends will be continually referenced as proximal end 304 and distal end 305 even if the orientation of suture 103 changes such that proximal end 304 no longer remains proximal to distal end 305 (e.g., as depicted in FIG. 5O).

FIG. 5B is another partial cross-sectional view depicting device 104 after snare head portion 152 has been advanced from within lumen 142 and into the open configuration. It should be noted that in this and subsequent figures, OA delivery members 401-1 and/or 401-2 may not be shown for purposes of clarity. In addition to being biased towards the open configuration, snare head portion 152 is also biased to fold over, or deflect, out of a housed configuration into a configuration oriented to facilitate capture of suture 103. For instance, here, snare head portion 152 deflects into an orientation in a plane substantially parallel with that of septal wall 207.

FIG. 5C is an end on view of septal wall 207 taken from left atrium 212 and showing snare end portion 152 in the open configuration. As can be seen here, snare head portion 152 includes a distal wedge-shaped “catch” portion 154 and a tapered proximal portion 155. Once fully deployed, needle 140 and snare 150 can be proximally retracted so that snare head portion 152 is approximately flush with septal surface 216, as depicted in the partial cross-sectional view of FIG. 5G.

FIG. 5D is a side view depicting an exemplary embodiment of needle 140 having slots 131 located near distal end 141. Slots 131 are preferably configured to receive snare body 151 as depicted in the frontal view of FIG. 5E. Slots 131 can be configured to be relatively dull (e.g., through electropolishing or the like) to minimize the risk of damaging snare body 151 with a sharp portion of the needle surface. FIG. 5F is an end on view of septal wall 207 taken from left atrium 212 showing this embodiment with snare end portion 152 in the open configuration and engaged with slots 131. Slots 131 can provide added stability to snare 150, as well as facilitate proper orientation of snare head portion 152 with respect to the desired penetration point 132 for needle 120.

Referring again to FIG. 5G, snare head portion 152 can be positioned against at least septum primum 214 to maintain primum 214 in a relatively fixed position. In this position, snare head portion 152 preferably encompasses the intended penetration point 132 for needle 120. Alternatively, snare head portion 152 can be oriented substantially parallel to the surface of septum primum 214 but not in actual contact with the primum tissue surface, i.e., remaining at a spaced distance from the tissue surface, which can also encompass the region of space adjacent to the intended penetration point for needle 120.

In order to facilitate the proper orientation of snare 150, the interior of lumen 142 can be configured to interface with snare base portion 153 in a manner that guides snare 150 into the proper orientation to position snare head portion 152 over the intended penetration point 132. For instance, in one exemplary embodiment, the radial surfaces of lumen 142 and snare base portion 153 are elliptical and only allow relative axial movement between needle 140 and snare 150 when in the properly aligned orientation. One of skill in the art will readily recognize that any complementary, non-circular shapes can be used for these surfaces to facilitate proper alignment. Furthermore, these configuration of surfaces can be used with any embodiment described herein where there is a need or desire to align two members that move in close proximity with respect to each other.

FIG. 5H depicts delivery device 104 after needle 120 has been advanced distally through septal wall 207 at location 132. The opening created by advancement of needle 120 through septal wall 207 is referenced here via numeral 136. Snare head portion 152 helps to maintain primum 214 in a fixed position during penetration by needle 120. As can be seen here, suture 103 is housed within an inner lumen 122 of needle 120. Suture body 301 has a proximal end 304 and a distal end 305. Proximal end 304 can include an anchor device 303 for anchoring proximal end 304 against surface 213 of septal wall 207. In this embodiment, anchor device 303 has a “T” configuration, which will be discussed in more detail below. It should be noted that in another exemplary embodiment, needle 120 can be omitted and closure element 103 can have a substantially sharp distal end and can be inserted through septal wall 207 to form opening 136.

After needle distal end 121 is exposed within left atrium 212, distal end 305 of suture 103 can be advanced from within needle lumen 122 using a flexible, elongate pusher member 128 that is slidably housed within lumen 122. This is depicted in FIG. 5I. The use of a pusher member 128 to deploy sutures and suture-like devices is described in detail in the above-referenced co-pending U.S. patent application entitled “Suture-based Systems and Methods for Treating Septal Defects” (Ser. No. 11/218,794).

Once suture distal end 305 is deployed, it can be captured with the aid of snare 150, as depicted in FIG. 5J. To begin the capture procedure, snare 150 can be proximally retracted with respect to needle 140 such that proximal tapered portion 155 approaches and abuts needle distal end 141. Because needle 140 is held in a relatively fixed position, the proximal force on tapered portion 155 causes snare head portion 152 to begin to swing back, or deflect, in direction 139 towards the original, relatively straightened configuration. As snare head portion 152 swings in direction 139, suture 103 begins to become entrapped by snare body 151 as shown. Also, because suture 103 is suspended within the fluid environment of left atrium 212, suture 103 resists being pulled along with body 151 and preferably becomes caught in the wedge-shaped distal “catch” portion 154 of snare 150. It should be noted that in this and other embodiments described herein, the proximal portion of needle distal end 121 (or needle distal end 141 where applicable) can be made relatively dull (e.g., by electropolishing and the like) to minimize the risk that suture 103 will be damaged during the capture and retrieval procedure.

As snare 150 continues to be proximally retracted into lumen 142, snare head portion 152 begins to return to the closed configuration, as depicted in FIG. 5K. This is facilitated by tapered proximal portion 155, which reduces the friction between snare head portion 152 and distal end 141 and eases the transition back to the closed configuration. FIG. 5L depicts another exemplary embodiment where a collet 162, mounted on the distal end of an elongate base member 163, is used to close snare head portion 152. Here, collet 162 is a rigid looped member that encompasses snare body 151. Collet 162 is sized to close snare head portion 152 as collet 162 is advanced over snare head portion 152, as depicted here. In another exemplary embodiment, collet 162 is configured as a slidable tubular member.

Return of snare 150 to the closed configuration causes distal catch portion 154 to compress around suture 103 and further tighten the grasp snare 150 acquires over suture 103. The frictional forces applied to suture 103 by nature of suture 103 being lodged and compressed within distal catch portion 154, as well as possibly being at least partially wrapped around snare body 151, act together in allowing snare 150 to capture suture 103, i.e., to grasp suture 103 by a degree sufficient to allow the application of force to suture 103 to eventually effect at least partial closure of tunnel 215. The force for closing tunnel 215 is then applied, preferably, by proximally retracting snare 150. Of course, snare 150 can be configured such that any of these frictional forces alone are sufficient to capture suture 103, or, alternatively, snare 150 can use different mechanisms or techniques to generate the force sufficient to capture suture 103. In addition, as will be discussed herein, suture 103 can be configured to further facilitate capture by snare 150 (e.g., by changing suture 103's diameter, shape, material and the like).

In FIG. 5M, snare 150 has been proximally retracted to draw suture 103 into needle lumen 142. Needle 140 and snare 150 have then been proximally retracted back through septal wall 207 together to “retrieve” suture 103, i.e., to bring suture 103 through opening 137 back into right atrium 205. Snare 150 is retracted with enough force to pull the entire excess portion of suture 103 present within left atrium 212 through opening 137. As depicted here, needle 120 has also been proximally retracted back through septal wall 207. Retraction of needle 120 can occur at any time after deployment of suture distal end 305, although preferably after suture distal end 305 has been retracted into right atrium 205 by snare 150. It can be desirable to retract needle 120 before suture 103 is pulled taught by snare 150 to avoid pulling suture 103 across distal end 121 of needle 120. Due to the removal of needles 120 and 140, the tissue surrounding openings 136 and 137 has closed in around suture 103. As a result of the penetrations, capture and retrieval, suture 103 is left routed from right atrium 205 through septal opening 137, across surface 216 (and over tunnel 215) and back to right atrium 205 by way of septal opening 136, in a position suitable to effect at least partial closure of tunnel 215.

If desired, delivery device 104 can be configured to maintain a grasp on suture proximal end 304 to prevent the entirety of suture 103 from being prematurely deployed during the capture and retrieval process. Devices and methods for maintaining grasp on proximal end 304 are discussed in detail in the incorporated co-pending application entitled “Suture-based Systems and Methods for Treating Septal Defects” (Ser. No. 11/218,794). In this embodiment, grasp is maintained with a grasping device (not shown) located on the distal end of pusher member 128. In this embodiment, grasp of suture proximal end 304 is maintained until suture 103 is captured and retrieved by snare 150, at which point suture proximal end 304 is released. Anchor device 303 can then be pulled flush with septal surface 213 by further retraction of snare 150.

After suture 103 is positioned as desired with anchor device 303 in contact with surface 213, a lock device 302 is preferably advanced from needle 140 and positioned over suture body 301 as depicted in FIGS. 5N-O. Here, lock device 302 is a compressible coil-like device configured to (1) compress or contract over suture body 301 to lock itself in position on body 301 and (2) abut septal surface 213 and resist being pulled through opening 137 to lock suture 103 in the position that effects at least partial closure, and preferably full closure of PFO tunnel 215. In this embodiment, lock device 302 is retained in an expanded state on the outer surface of needle 140. Deployment of lock device 302 is achieved by advancing the distal end 130 of a tubular pusher member 129 against lock device 302 (as depicted in FIG. 5N) to slide device 302 off of needle distal end 141 and onto suture body 301 in the desired position (depicted in FIG. 5O). Alternatively, or in combination, needle 140 can be retracted proximally relative to pusher member 129 to deploy lock device 302. Continuing advancement of pusher member 129 against lock device 302 while proximal tension is maintained on suture body 301 will apply additional closure force. Preferably, to apply that additional closure force, lock device 302 includes lateral arms, or petals. The many different types of lock devices 302 and methods of deploying them are discussed in further detail below.

Once lock device 302 is deployed, a cutting device (not shown) can be used to free suture 103 from snare 150 and to trim any excess portion of suture body 301 present within right atrium 205. Cutting elements are well known to those of ordinary skill in the art and any type of cutting element can be used as desired. For instance, the cutting element can be placed on the proximal end or other location of the lock device 302, or the cutting device can be a slot in needle 140 having a substantially sharp edge, or additional mechanical cutting elements and devices can be used. Heat or energy-based cutting devices can also be used, by themselves or in conjunction with mechanical cutting. Delivery device 104 can then be removed from heart 200 leaving suture 103 deployed over and within septal wall 207 in a position constricting and preferably fully closing PFO tunnel 215, as depicted in FIG. 5P.

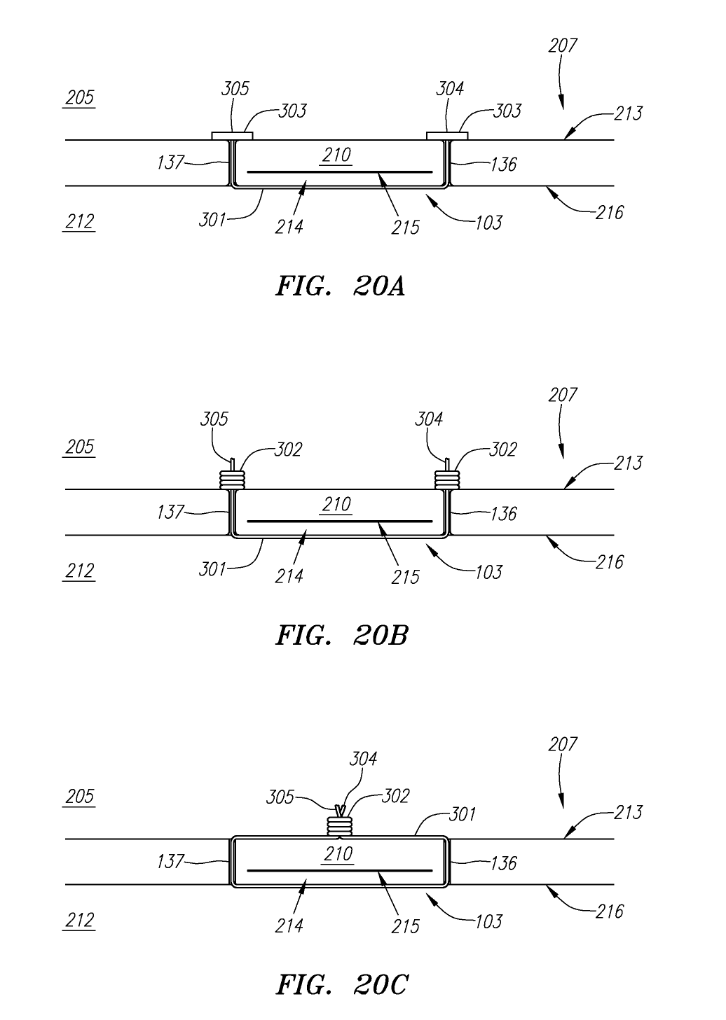

It should be noted that, although in this embodiment closure device 103 is fixed in place using a lock device 302 and an anchor device 303 placed on opposite ends, in the various embodiments described herein, closure device 103 can be fixed in place using any combination of lock devices 302 and anchor devices 303. For instance, each end of the closure device 103 can be configured with an anchor device 303 (e.g., see FIG. 20A), or each end of closure device 103 can be locked in place using a lock device 302 (e.g., see FIG. 20B), or any combination thereof. Also, a single lock device 302 can be used by placing it over both ends of closure device 103, as will be described herein (e.g., see FIG. 20C). Furthermore, an end of closure device 103 can be configured with both a lock device 302 and an anchor device 303 for redundancy.

Referring back to FIG. 5A, it should be noted that the desired locations 132 and 133 where needles 120 and 140, respectively, are intended to be inserted into septal wall 207 can be varied as needed, depending on the size and orientation of PFO tunnel 215, the type of closure element 103 being used, etc. For instance, FIGS. 5Q-R are perspective views taken from right atrium 205 depicting several different intended locations 132 and 133 for insertion. In FIG. 5Q, locations 132 and 133 are oriented diagonally across tunnel 215, while in FIG. 5R locations 132 and 133 are located within tunnel 215 such that needles 120 and 140 penetrate both septum secundum 210 and septum primum 214. Points 132 and 133 can also be outside of tunnel 215, as well as any combination of these inside tunnel 215, outside tunnel 215, diagonally oriented and the like.

In addition to varying the intended locations 132 and 133 for needle insertion, the angle at which needles 120 and 140 are inserted through septal wall 207 can be varied as well. For instance, FIGS. 5S-T are partial cross-sectional views depicting exemplary embodiments where needles 120 and 140 are inserted into septal wall 207 at angles 134 and 135, respectively. Angles 134-135 are measured absolute with respect to a normal 224 to septal wall 207. In this embodiment, angles 134-135 are each approximately 30 degrees, although it should be noted that any angular values less than or equal to ninety degrees can be used.

FIG. 5U is a radial cross-sectional view depicting another exemplary embodiment of system 100 where two off-axis delivery members 401 are configured to deflect at an angle 403 with respect to each other to create openings in the septal tissue that are preferably spaced apart and oriented transverse to each other, such as that described with respect to FIG. 5T. In this embodiment, two OA members 401-1 and 401-2 are slidably received within body member 101 in a single lumen, or in separate lumens 405-1 and 405-2 as depicted here. Arm members 409-1 and 409-2 are coupled with body member 101 and oriented at angle 403 such that when OA members 401-1 and 401-2 deflect outwards, or arc upwards, to capture septal tissue and/or enter the “off-axis” configuration, members 401-1 and 401-2 do so in directions 406-1 and 406-2, respectively. This orients OA members 401-1 and 401-2 such that needle members 120 and 140 will be advanced towards each other, although the OA members 401-1 and 401-2 can be staggered (i.e., the axial positions of distal ends 410-1 and 410-2 along the longitudinal axis of body member 101 are different) to decrease the chance that needle members 120 and 140 will hit each other when advanced. Also, OA members 401 can have different degrees of offset to vary the angles 134-135 (not shown) at which needles 120 and 140 are oriented with respect to the tissue surface. (Here, two arm members 409 are coupled with each OA member 401.)

In this and other embodiments described herein, two needles or piercing elements are used to puncture septal wall 207 to gain access to the opposing atrial chamber. It should be noted that one of these two punctures can, in appropriate circumstances, be avoided by instead using the natural PFO tunnel 215 to access the opposing atrial chamber.

FIGS. 6A-E are partial cross-sectional views depicting another exemplary embodiment of system 100 where needle 140 is configured to curve to allow penetration of septal wall 207 in both a distal and proximal direction. In this embodiment, needle 140 is configured to curve approximately 180 degrees into a “J” shape. Design and configuration of curved needles to penetrate septal wall 207 is described in further detail in the incorporated patent application entitled “Suture-based Systems and Methods for Treating Septal Defects” (Ser. No. 11/218,794).

FIG. 6A depicts delivery device 104 after needle 140 has been advanced distally through septal wall 207. In this embodiment, a distal portion of needle 140 is biased to enter the curved configuration and is kept in the straightened configuration by maintaining it within an inner lumen 124 of a relatively rigid tubular outer member 123. Once passed through septal wall 207, needle 140 can be advanced distally with respect to outer tubular member 123 to expose the distal portion of needle 140 and allow it to enter the curved “J” configuration, as depicted in FIG. 6B. In this curved configuration, needle 140 can be retracted proximally to pass needle distal end 141 back through septal wall 207. As a result, needle 140 openings 136 and 137 are both created by needle 140, as depicted in FIG. 6C.

Next, snare 150 can be advanced from within needle inner lumen 142 in preparation for capturing suture 103. Member 120, which is not configured as a needle in this embodiment, is preferably advanced into proximity with snare head portion 152, at which point suture 103 can be advanced through distal end 127, as depicted in FIG. 6D. Snare 150 is then preferably used to capture suture 103 and pull it into needle lumen 142. Needle 140 can then be advanced distally to pass needle distal end 142 back through opening 136. Needle 140 can then be returned to the substantially straightened configuration by retracting it back into lumen 124 of tubular member 123, as depicted in FIG. 6E (depicting needle 140 partially straightened). Once needle 140 is fully retracted into lumen 124, both needle 140 and member 123 can be retracted proximally through opening 137, leaving suture 103 routed through openings 136 and 137 as depicted in FIG. 6F. Anchor device 303 (not shown) can be released from member 120 and the opposite end of suture 103 can be locked on right atrial tissue surface 213 with lock device 302 (not shown) to complete the closure procedure, in a manner similar to that described with respect to FIGS. 5J-M above.

In addition to member 140, member 120 and other components of system 100 can also be configured to curve to facilitate capture of suture 103. For instance, FIG. 6G is a partial cross-sectional view depicting an exemplary embodiment where member 120 is configured to curve in a manner similar to member 140 as described with respect to FIGS. 6A-F above. Here, member 120 is a needle-like member configured to curve into a “J” shape and is used with tubular member 123. Needle 120 is shown after being advanced through septal wall 207 in two separate locations to form openings 136 and 137. Suture 103 has been advanced from within needle lumen 122 and through snare head portion 152, which is in an open, deflected state, deployed from dull distal end 143 on member 140 (which in this embodiment is not configured as a needle). From here, suture 103 can be captured and retrieved by snare 150 and needle 120 can be withdrawn from septal wall 207 to complete the closure procedure.

FIGS. 6H-I are partial cross-sectional views depicting two additional exemplary embodiments of delivery device 104 with curved components. FIG. 6H depicts needle 120 in a curved state where distal end 121 is curved to one side by approximately ninety degrees. Needle 140 is used to create septal opening 137 and needle 120 does not need to curve into the full 180 degree “J” configuration depicted in FIG. 6G. Snare 150 has been advanced from within needle lumen 142 and is in position to capture suture 103, which has been advanced from within needle lumen 122. From here, suture 103 can be captured and retrieved and needles 120 and 140 can be withdrawn from septal wall 207 to complete the closure procedure. In a preferred embodiment, needle 140 is withdrawn prior to needle 120.

FIG. 6I depicts needle 120 and needle 140 each in a curved state where distal ends 121 and 141 are deflected towards each other. In this embodiment, the amount of deflection in both needles 120 and 140 is approximately ninety degrees and each is used in conjunction with tubular members 123-1 and 123-2, respectively. Like in FIGS. 6G-H, snare 150 has been advanced from within needle lumen 142 and is in position to capture suture 103, which has been advanced from within needle lumen 122. From here, suture 103 can be captured and retrieved and needles 120 and 140 can be withdrawn from septal wall 207 to complete the closure procedure.

FIGS. 6J-N depict an additional exemplary embodiment of delivery device 104. Here, needles 120 and 140 are each configured to curve at an angle of approximately forty-five degrees, similar to the embodiment described with respect to FIG. 6I. In this embodiment, a portion of snare 150 is detachable and configured to form part of the closure element, which includes suture 103 as well as the detached portion of snare 150. Suture distal end 305 is configured to engage with or grasp snare body 151 in snare head portion 152 and can be curved or bent into a rigid configuration, or can include an anchor-type device 303, in a manner similar to the embodiments described with respect to FIGS. 19A-C.

FIG. 6J is a partial cross-sectional view depicting delivery device 104 after suture 103 has been captured by snare head portion 152. Here, suture distal end 305 is bent in a hook-like configuration. FIG. 6K is another partial cross-sectional view depicting delivery device 104 after needles 120 and 140 have been proximally retracted through septal wall 207. FIG. 6L is a side view showing septal wall 207 as viewed from the left atrium. Here, suture 103 can be seen engaged with snare 150. Limbus 211 and PFO tunnel sidewalls 219 are referenced with dashed lines to indicate their obstruction by septum primum 214. Here, suture 103 has been proximally retracted such that distal end 305 is the only portion of suture 103 exposed in left atrium 212 and snare head portion 152 preferably extends over the majority of primum 214. FIG. 6M is another side view showing an exemplary embodiment where snare 150 and a relatively greater portion of suture 103 both extend over primum 214.

Once needles 120 and 140 are proximally retracted, lock devices 302 can be applied over suture 103 and snare base portion 153, as depicted in FIG. 6N. Once the lock devices 302 are deployed, suture 103 can be severed and a distal portion of snare 150, including head portion 152 and a part of base portion 153, can be detached. Detachment of snare 150 can be accomplished using a cutting device (not shown), such as that used with suture 103, or in an alternative embodiment, snare 150 can be held together with a mechanical locking mechanism.

FIG. 6O is a cross-sectional view depicting an exemplary embodiment of delivery device 104 with snare 150 configured for mechanical detachment. Here, snare head portion 152 is coupled with snare base portion 153 in an interlocking region 196, where snare body 151 is relatively thicker than in the adjacent regions. Proximal end 197 of snare head portion 152 is configured to interlock with distal end 198 of snare base portion 153 and can have a shape complementary to that of distal end 198. Interlocking region 196 is preferably sized so as to remain in the locked state while within inner lumen 142 of needle 140. Upon retraction of needle 140, interlocking region 196 becomes exposed and snare head portion 152 is free to detach from snare base portion 153. Other configurations can also be used, such as a ball a socket interface (not shown). One of skill in the art will readily recognize the many different manners in which attachment/detachment can be achieved.

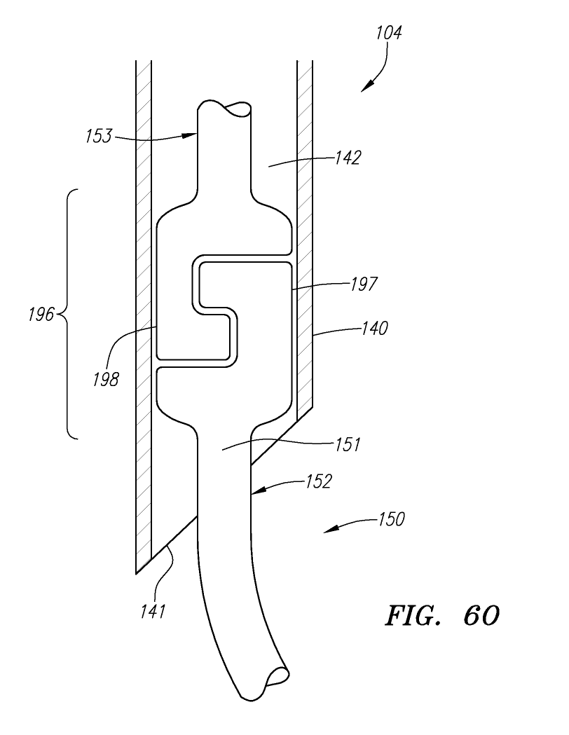

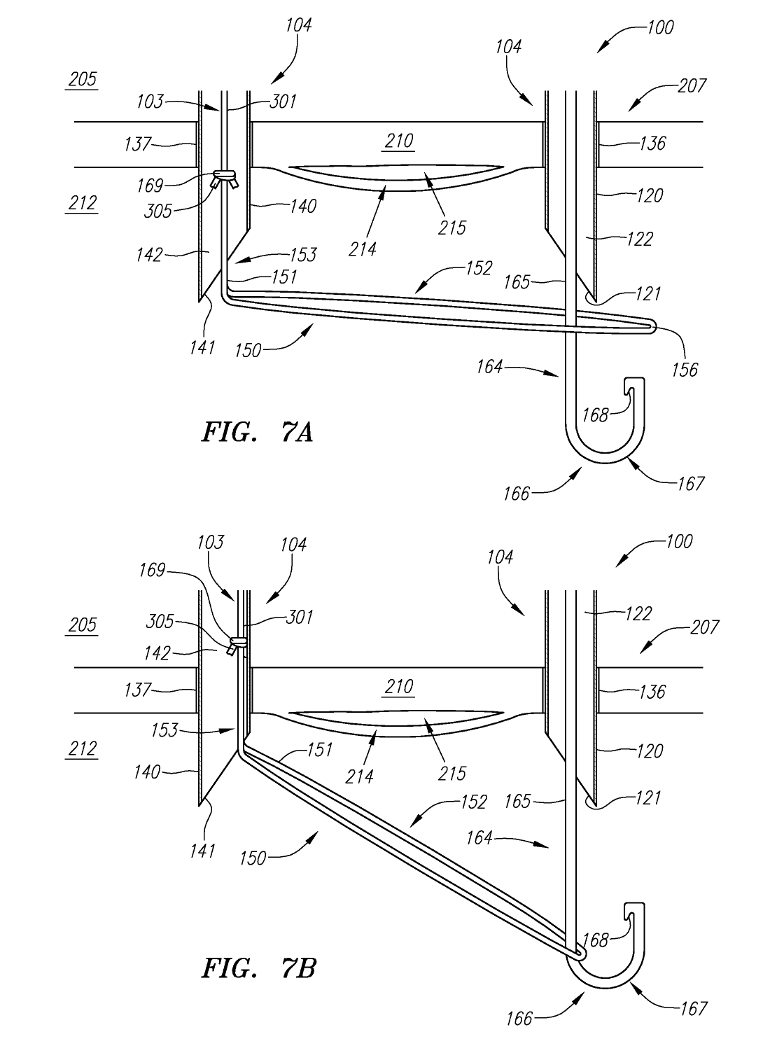

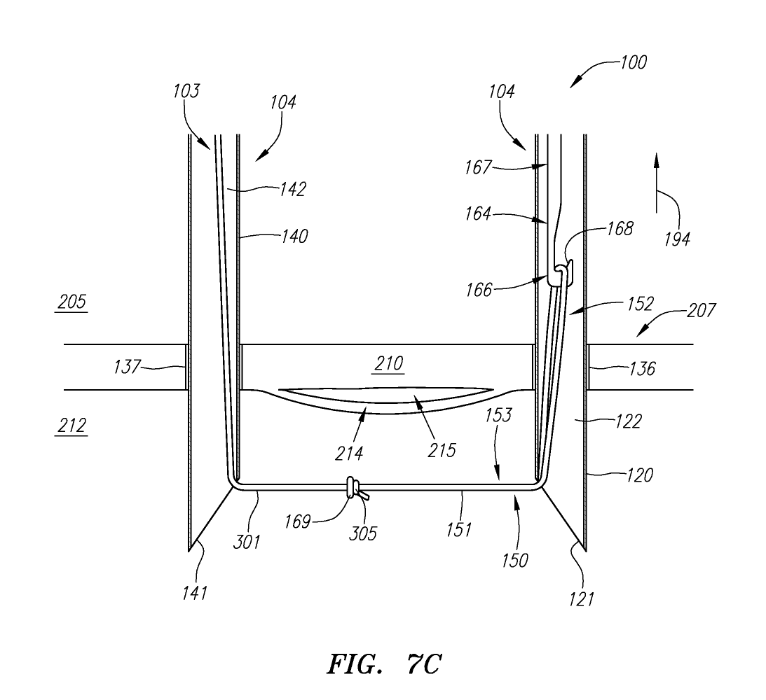

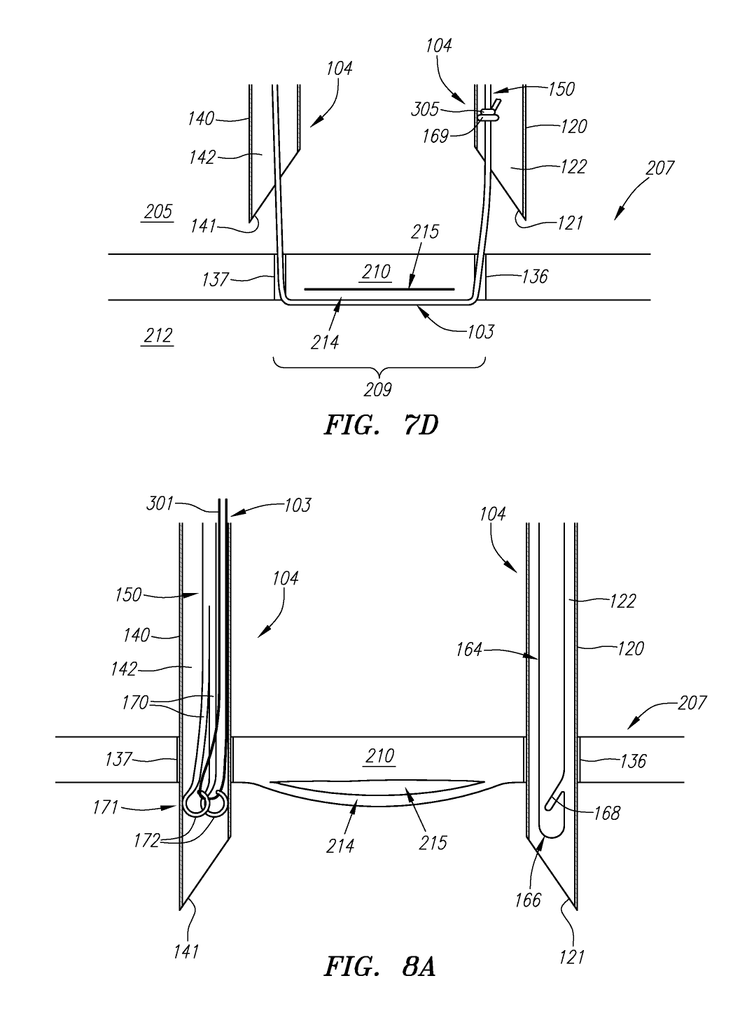

Instead of using snare 150 as a vehicle to capture suture 103, snare 150 and suture 103 can be combined, or integrated, and used in conjunction with an additional capture device to draw suture 103 across septal wall 207. FIG. 7A is a partial cross-sectional view depicting one exemplary embodiment of system 100 configured to use an additional capture device 164. Here, needles 120 and 140 have been advanced through septal wall 207 to create punctures 136 and 137, respectively, and capture device 164 has been partially advanced from lumen 122 through distal end 121. Capture device 164 can include a flexible elongate body 165 and a distal end 166 having a curved, hook-like portion 167 with a notch 168. Capture device 164 is preferably deflectable between the configuration depicted here, where portion 167 is in the hook-like state, and a relatively straightened configuration where capture device 164 can reside entirely within lumen 122 of needle 120. Portion 167 is preferably biased towards and enters the hook-like state upon deployment from distal end 121. Capture device 164 is preferably fabricated from a flexible, biocompatible material such as NITINOL, although any other desired material can be used including stainless steel, elgiloy and the like.

As depicted here, snare 150 has been advanced distally from within inner lumen 142 of needle 140 allowing snare head portion 152 to deploy and enter the open configuration and preferably deflect over capture device 164. From this configuration, capture device 164 can be freely positioned to capture snare 150. It should be noted that capture device 164 and snare 150 can be implemented in any of many alternative configurations that can also allow capture. For instance, snare 150 can be configured to deflect over needle 120 prior to deployment of capture device 164, such that after advancement of capture device 164, needle 120 can be retracted and then snare 150 can be proximally retracted to effect capture with capture device 164. Also, snare 150 can be configured to exit lumen 142 without deflecting, while capture device 164 can be configured to deflect towards snare 150. In another alternative, both snare 150 and capture device 164 can be configured to deflect towards each other. In each of these cases, either device can be advanced in any desired order to allow capture.

In this embodiment, proximal end 169 of snare 150 is coupled with distal end 305 of suture 103 such that the grasping and pulling of snare 150 will draw snare 150 and suture 103 out of inner lumen 142. Snare 150 is preferably advanced by applying force in a distal direction to suture body 301. In embodiments where the stiffness of suture body 301 is insufficient to permit snare 150 to be advanced, an additional pushing member (not shown) can be used such as an elongate member slidably disposed within inner lumen 142.

FIG. 7B depicts this exemplary embodiment after capture device 164 has captured snare 150. At this point either one or both devices can be proximally retracted to remove any slack and preferably create a “snug” engagement. FIG. 7C depicts this exemplary embodiment after capture device 164 has been retracted proximally into inner lumen 122. Upon retraction, snare 150 preferably engages, or catches, notch 168, such that capture of snare 150 can be maintained as curved portion 167 is deflected back into the relatively straightened state upon retraction. Continued retraction of capture device 164 draws snare 150 into inner lumen 122 with suture 103.

FIG. 7D depicts this exemplary embodiment after capture device 164 (not shown) and snare 150 have been fully retracted into inner lumen 122 and needles 120 and 140 have been withdrawn from septal wall 207. At this point, suture 103 is routed entirely through septal wall on both sides of, or at multiple locations within, PFO region 209. Although not shown, the opposite ends of suture 103 can then be fastened against septum secundum 210 using a single lock device 302, for instance, deployed over both needles 120 and 140, or with any other alternative technique described herein (e.g., use of a separate lock device 302 on either end, use of anchor device 303 on proximal end 304 of suture 103 with a lock device on the opposite end, and the like) or readily apparent to one of ordinary skill in the art. After fastening suture 103, any excess portion of suture body 301 can be trimmed using any desired mechanical technique (e.g., using a sharp edge), thermal technique (e.g., resistance heated wire via electrical energy) or the like.

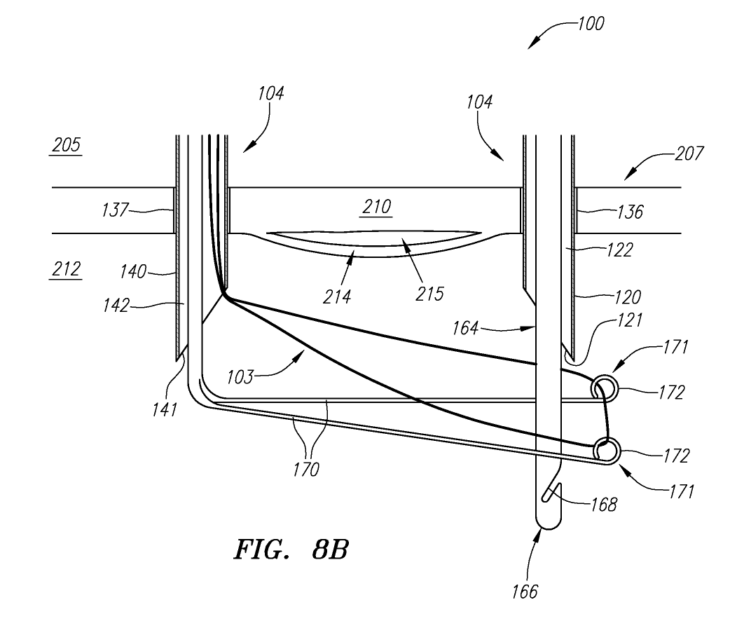

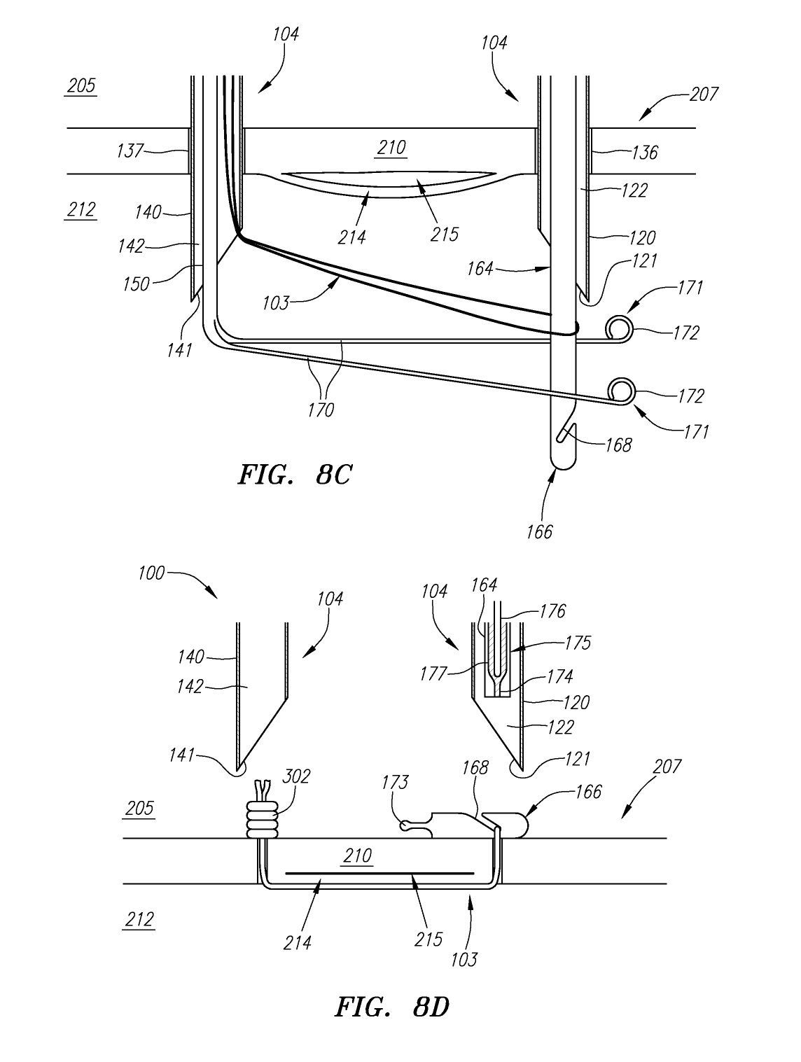

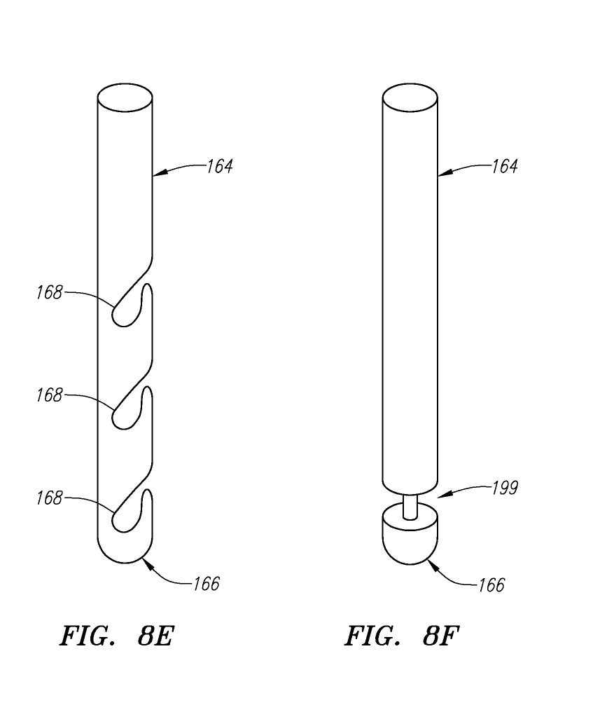

FIG. 8A is a partial cross-sectional view depicting yet another exemplary embodiment of system 100 with snare 150 again integrated with suture 103. Snare head portion 152 can be configured as a frame, with one or more arms 170 configured to hold or present suture 103 in a position suitable for capture with capture device 164. Each arm 170 has a distal end 171 with a holding element 172 configured to releasably hold suture body 301.

FIG. 8B depicts this exemplary embodiment after advancement of snare 150 from within inner lumen 142. Upon deployment from lumen 142, arms 170 are preferably biased to enter an orientation such that distal ends 171 are spaced apart and deflected over capture device 164 with suture 103 extending therebetween (e.g., in a “Y” configuration as depicted here). In this embodiment, holding elements 172 are flexible, curled elements configured to hold suture 103 therein. Holding elements 172 are deflectable upon application of tension to suture 103 such that suture 103 can be released from holding elements 172. It should be noted that any suitable configuration can be used for holding elements 172 including, but not limited to shapes such as hooks, coils, clamps, configurations that use magnetic forces, configurations that use adhesives, and the like. Also, each arm 170 can include more than one holding element 172 located at various locations along the length of the arm 170. Arms 170 can be relatively straight, as depicted in FIG. 8B, or can be curved or bent, to increase the size of the region of space surrounded by suture 103 in which capture device 164 is preferably positioned.

In this configuration, capture device 164 and snare 150 can be manipulated in multiple ways such that capture device 164 captures suture 103 from holding elements 172. In the exemplary embodiment depicted in FIG. 8B, capture device 164 has been advanced distally into the opening between arms 170 such that notch 168 lies distal to suture 103. In FIG. 8C, suture 103 has been proximally retracted with respect to snare 150 to release suture 103 from holding elements 172 and place suture 103 in a relatively snug manner around capture device 164. Capture device 164 can then be proximally retracted to cause notch 168 to capture suture 103 and draw it into inner lumen 122 and snare 150 can be proximally retracted back into needle 140. Each needle 120 and 140 can then be withdrawn from septal wall 207, leaving suture 103 in position to at least partially close PFO tunnel 215.

In an alternative exemplary embodiment, instead of retracting suture 103 to free it from holding elements 172, snare 150 can be retracted to cause suture 103 to come into contact with capture device 164. Capture device 164 can then be proximally retracted to capture suture 103 in notch 168 and pull suture 103 from holding elements 172.

Suture 103 can then be fastened against septum secundum 210 with a lock device 302 or an anchor device 303 in a manner similar to the embodiments described herein. In another exemplary embodiment, distal end 166 of capture device 164 can be detachable so as to form an anchor for one side of suture 103, as depicted in FIG. 8D. Here, detachable distal end 166 has a ball-like prong 173 configured to detach from a socket 174 in the proximal portion 175 of capture device 164. Distal end 166 can be detached with the aid of a pusher member 176 slidable disposed within a central lumen 177 of capture device 164. The opposite portion of suture 103 is shown here trimmed and fastened against septal wall 207 with coiled lock device 302.

In another exemplary embodiment, capture device 164 can be configured to capture suture 103 while still retained by holding elements 172. Capture device 164 can then be used to pull suture 103 from holding elements 172. For instance, capture device 164 can include curved distal portion 167 configured to deflect upon advancement from needle 120 and grasp suture 103 in a manner similar to that described with respect to FIGS. 7A-D. In general, it should be noted that capture device 164 and/or snare 150 can be configured to deflect in order to facilitate capture.

FIGS. 8E-F are perspective views depicting additional exemplary embodiments of capture device 164. In FIG. 8E, capture device 164 includes multiple notches 168 disposed with the same orientation along the axial length of capture device 164. Alternatively, each notch 168 can be oriented differently, to allow capture of suture 103 with less regard to the radial orientation of capture device 164. FIG. 8F depicts an embodiment where capture device 164 includes a circumferential groove 199 that allows capture of suture 103 without any regard for the radial orientation of capture device 164. It should be noted that any number of circumferential grooves can be used.

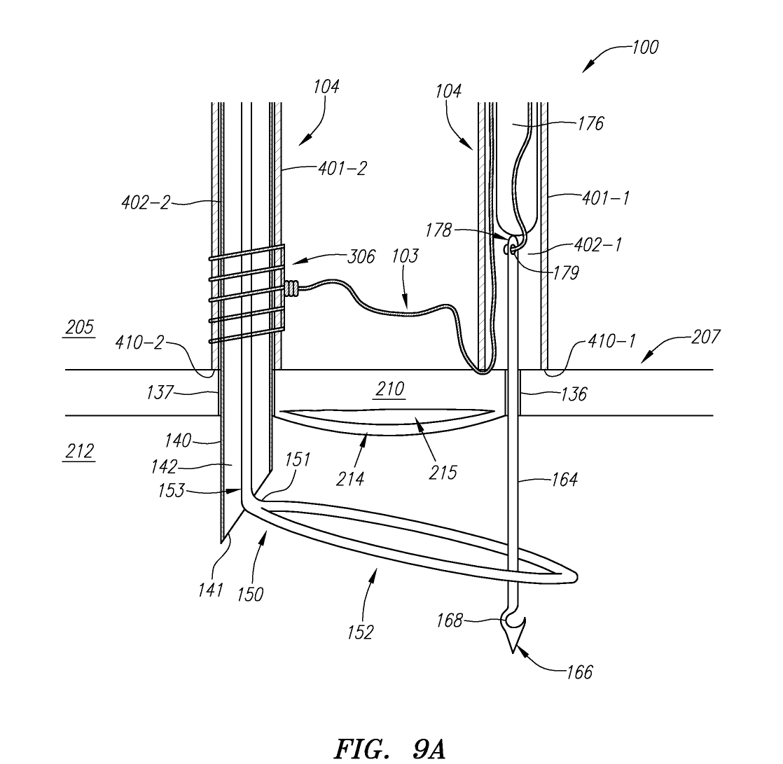

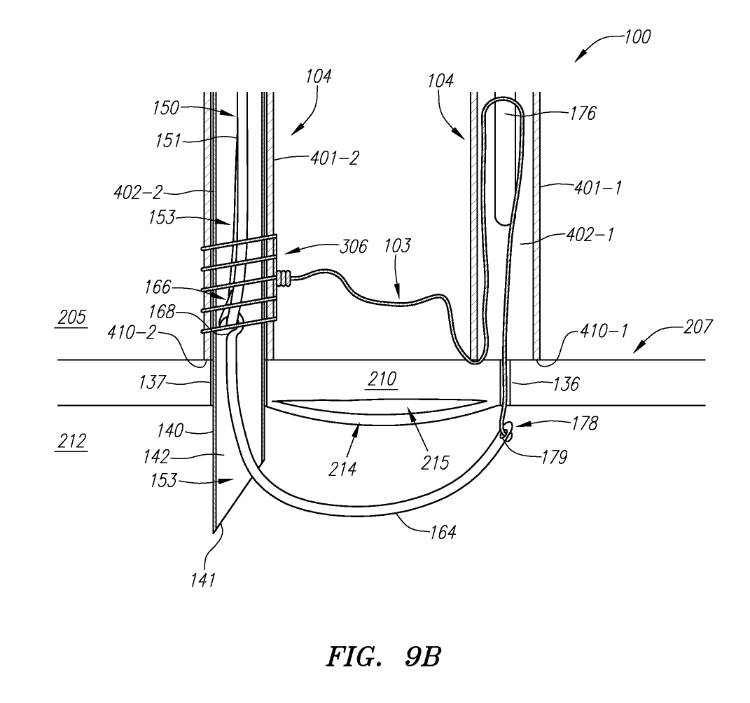

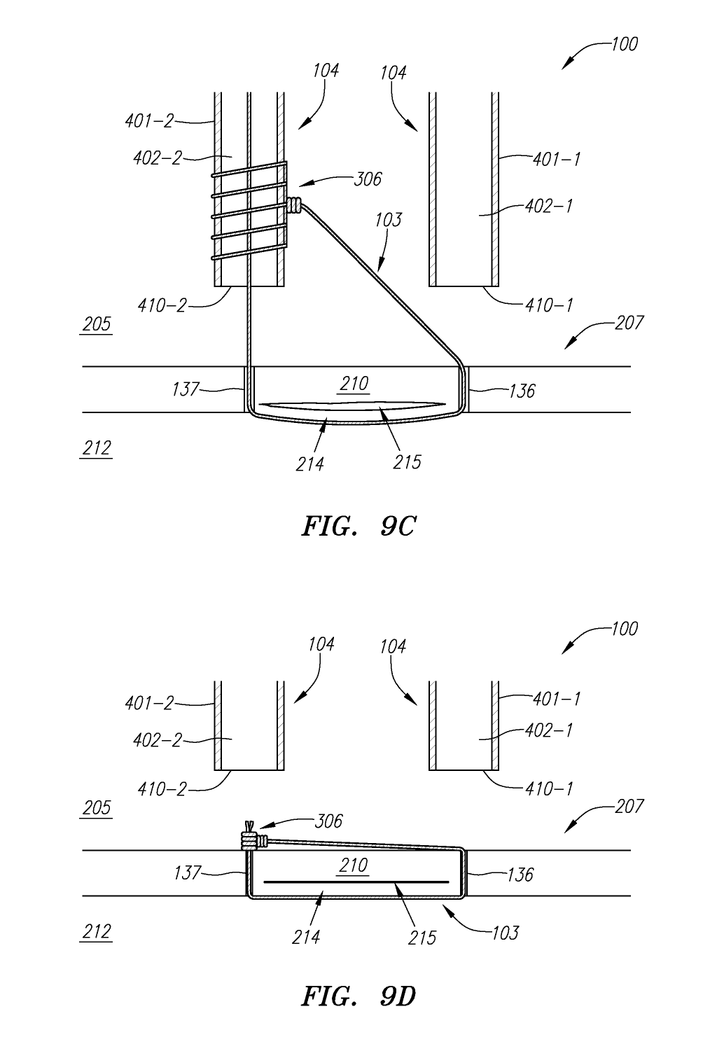

FIGS. 9A-D are partial cross-sectional views depicting another exemplary embodiment of system 100. In this embodiment, snare 150 is configured to draw capture device 164 into snare needle lumen 142. Needle 120 can be omitted and capture device 164 can instead be configured with a substantially sharp distal end 166 configured to penetrate septal wall 207. Pusher member 176 can be used to push against proximal end 178 of capture device 164 and drive capture device 164 through septal wall 207. A first end of suture 103 is preferably coupled with proximal end 178 of capture device 164, such as through aperture 179, although any manner of coupling can be used. In this embodiment, capture device 164 is a generally flexible, wire-like element with distal end 166 being relatively thicker to allow for the incorporation of notch 168. The generally flexible configuration facilitates the ability to draw capture device 164 into snare needle lumen 142. However, capture device 164 is preferably configured with sufficient rigidity or columnar strength to allow advancement through the desired portion of septal wall 207 without bending or breaking. The opposite end of suture 103 can be placed in a slipknot-type configuration 306 about the exterior of OA delivery member 401-2, with the main portion of suture 103 housed within inner lumen 402-1 of OA delivery member 401-1.

FIG. 9A depicts capture device 164 and needle 140 after advancement through septal wall 207 with snare 150 also having been advanced to allow snare head portion 152 to deflect over distal end 166 of capture device 164. From this position, snare 150 is preferably proximally retracted to engage with notch 168 of capture device 164. After engagement, snare 150 can be further retracted to draw capture device 164 into needle lumen 142, as depicted in FIG. 9B. This action pulls suture 103 through septal wall 207. Once capture device 164 has been drawn into needle lumen 142 by the desired amount, needle 140 and snare 150 can be proximally retracted into inner lumen 402-2 of OA delivery member 401-2.

Snare 150 is preferably retracted continuously until only suture 103 is exposed from distal end 410-2 of OA delivery member 401-2 and suture 103 has been pulled completely from within lumen 402-1 of OA delivery member 401-1, as depicted in FIG. 9C. Here, OA delivery members 401-1 and 401-2 have been retracted away from septal wall 207. Continued retraction of OA delivery member 401-2 and/or continued retraction of snare 150 preferably causes slipknot configuration 306 to be pulled from the exterior of OA delivery member 401-2 and to tighten around suture 103 against septal wall 207.

FIG. 9D depicts this embodiment with slipknot 306 tightened around suture 103. Suture 103 can then be cut or otherwise separated to leave PFO tunnel 215 in an at least partially closed state as depicted here. It should be noted that instead of tying suture 103 in slipknot configuration 306, suture 103 can be simply looped around the exterior of OA delivery member 401-2 such that a lock device 302 can be placed over suture 103 against septal wall 207 once suture 103 is tightened by the desired amount. Or, alternatively, suture 103 can be coupled to a lock device 302 located over OA member 401-2, where the lock device 302 is configured to compress over suture 103 not dissimilar to that of a slipknot. Lock device 302 could be configured as an elastic band or coil, to name a few examples.

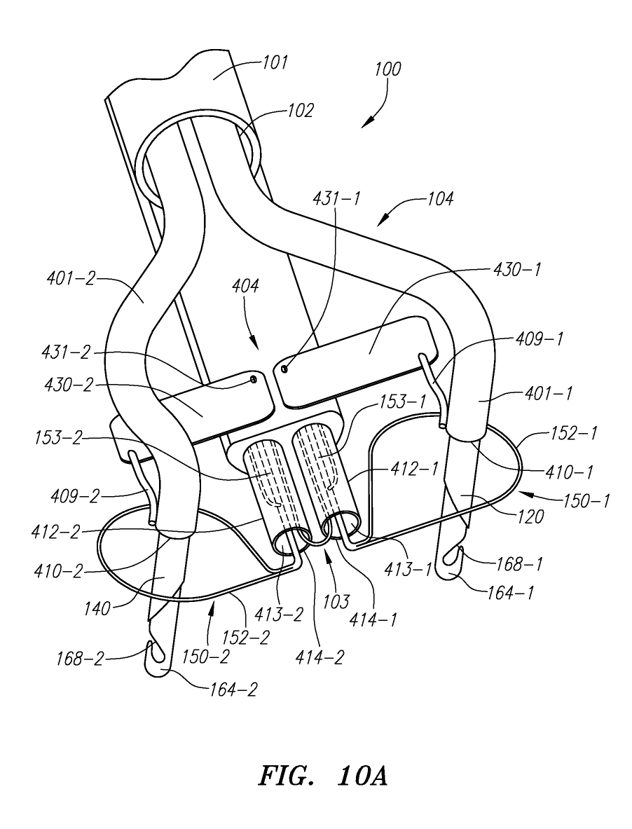

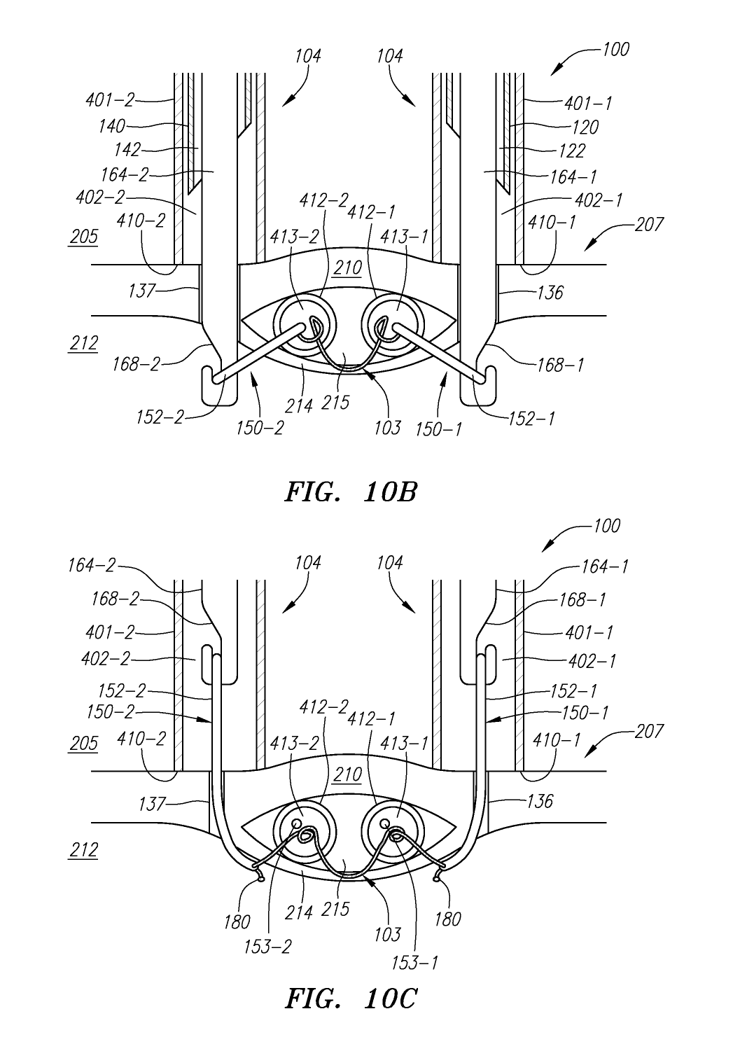

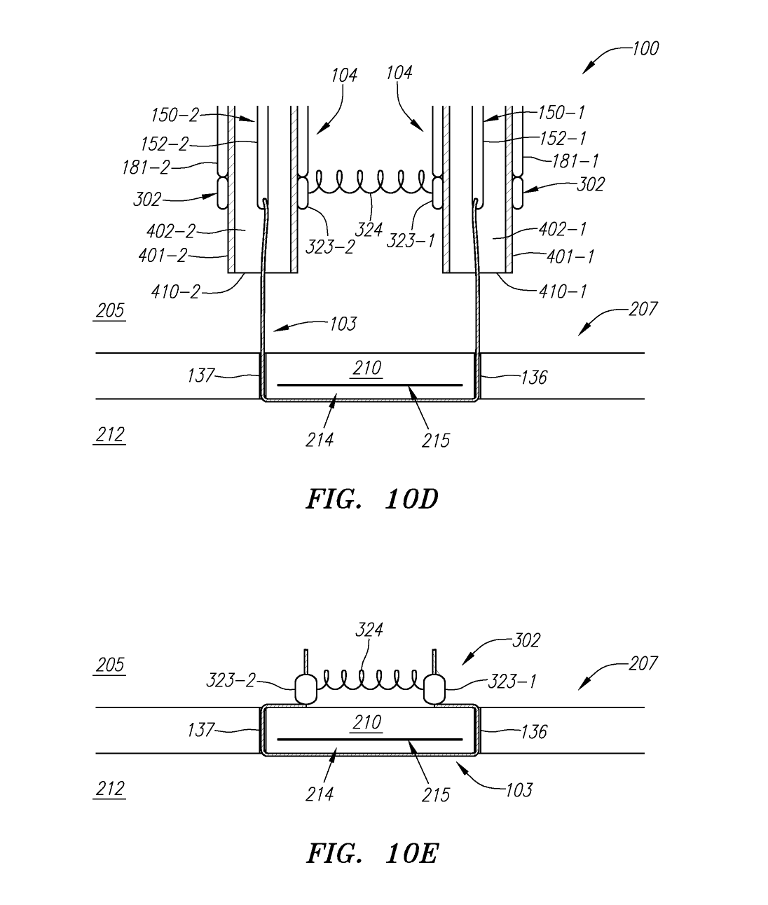

FIGS. 10A-E depict another exemplary embodiment of system 100 where snare 150 is integrated with a suture 103. In this embodiment, opposing ends of suture 103 can be coupled with two snares 150-1 and 150-2, preferably snare head portions 152-1 and 152-2, which can each in turn be captured with capture devices 164-1 and 164-2, respectively.

FIG. 10A is a perspective view depicting this exemplary embodiment during an exemplary treatment procedure (septal wall 207 is not shown for clarity). Here, delivery device 104 is similar to the embodiment described with respect to FIG. 3E with several differences. In place of elongate support structure are two elongate tubular members 412-1 and 412-2. Tubular members 412 are preferably configured to be inserted into native PFO opening 215. Each tubular member 412 includes an inner lumen 413 and an open distal end 414 and is configured to slidably house snares 150 and suture 103 such that suture 103 can reside between each tubular member 412. Members 412 can be fixably coupled to body member 101 (as shown), or members 412 can be slidably disposed within lumens in body member 101.

Here, snares 150 are shown with snare head portion 152 deployed from tubular members 412 into the open configuration. Snares 150 are preferably configured such that snare head portions 152 are detachable from the proximal snare base portions 153 (shown to be within lumens 413 with dashed lines). OA delivery members 401-1 and 401-2 are shown in the off-axis configuration with needles 120 and 140 extended from within. Capture devices 164-1 and 164-2 are likewise shown extended from within needles 120 and 140, respectively. Preferably, delivery device 104 is configured such that needles 120 and 140 and/or capture devices 164-1 and 164-2 will extend through snares 150-1 and 150-2, respectively, when in the configuration shown here.

FIG. 10B is a partial cross-sectional view depicting the exemplary embodiment of FIG. 10A during an exemplary treatment procedure. At this point in the procedure, needles 120 and 140 have been proximally retracted back through septal wall 207 and into inner lumens 402-1 and 402-2, respectively. Snares 150-1 and 150-2 have also been proximally retracted partially into lumens 413-1 and 413-2 respectively, such that each snare head portion 153 has tightened around the respective capture device 164. Capture devices 164-1 and 164-2 can then be proximally retracted to capture snare head portions 152-1 and 152-2 within notches 168-1 and 168-2, respectively.

FIG. 10C depicts this embodiment after capture devices 164 have been proximally retracted into lumens 402. Proximal retraction of capture devices 164 preferably causes snare head portions 152 to detach from snare base portions 153. The detachment mechanism 180 can be configured in any desired manner, preferably one that resists detachment to a degree sufficient to allow distal and proximal movement of snare 150 without resulting in detachment of head portion 152. For instance, detachment mechanism 180 preferably does not detach in the instance that the user decides to retract snare head portions 152 without capture devices 164 in the proper position. This can allow the procedure to be aborted if desired. In this embodiment, detachment mechanism 180 includes a ball and socket type configuration.

As shown here, retraction of capture devices 164-1 and 164-2 likewise draws snare head portions 152-1 and 152-2 through openings 136 and 137 and into inner lumens 402-1 and 402-2, respectively. As this occurs, suture 103 is pulled from within lumens 413. FIG. 10D depicts this exemplary embodiment after suture 103 has been pulled entirely from within lumens 413. Members 412 are preferably retracted from PFO tunnel 215, to allow suture 103 to draw tunnel 215 at least partially closed. Also, snare head portions 152 have been retracted entirely within OA delivery members 401, which in turn have been removed from septal wall 207 leaving suture 103 routed around PFO tunnel 215. Lock device 302 is shown about the exterior of OA delivery members 401-1 and 401-2.

Once in the position shown here, lock device 302 can be advanced off of OA delivery members 401 and over suture 103. Lock device 302 is preferably configured to contract or otherwise tighten around suture 103 to lock suture 103 in place, as depicted in FIG. 10E. In this embodiment, lock device 302 can be configured with two compressible cuffs 323-1 and 323-2 with an optional bias member 324 coupled therebetween. Cuffs 323-1 and 323-2 can be any compressible device, such as an elastic band, a NITINOL coil and the like, and are preferably advanced off of OA delivery members 401-1 and 401-2 with tubular members 181-1 and 181-2, respectively, although other types of lock device deployment mechanisms can be used. Cuffs 323-1 and 323-2 then preferably compress around the opposite ends of suture 103, while bias member 324, which in this embodiment is configured as a spring, exerts a bias force on each cuff 323-1 and 323-2 to draw them together and provide additional closure force. Suture 103 can then be released or cut to separate it from OA delivery members 401, leaving PFO tunnel 215 at least partially, and preferably entirely, closed.

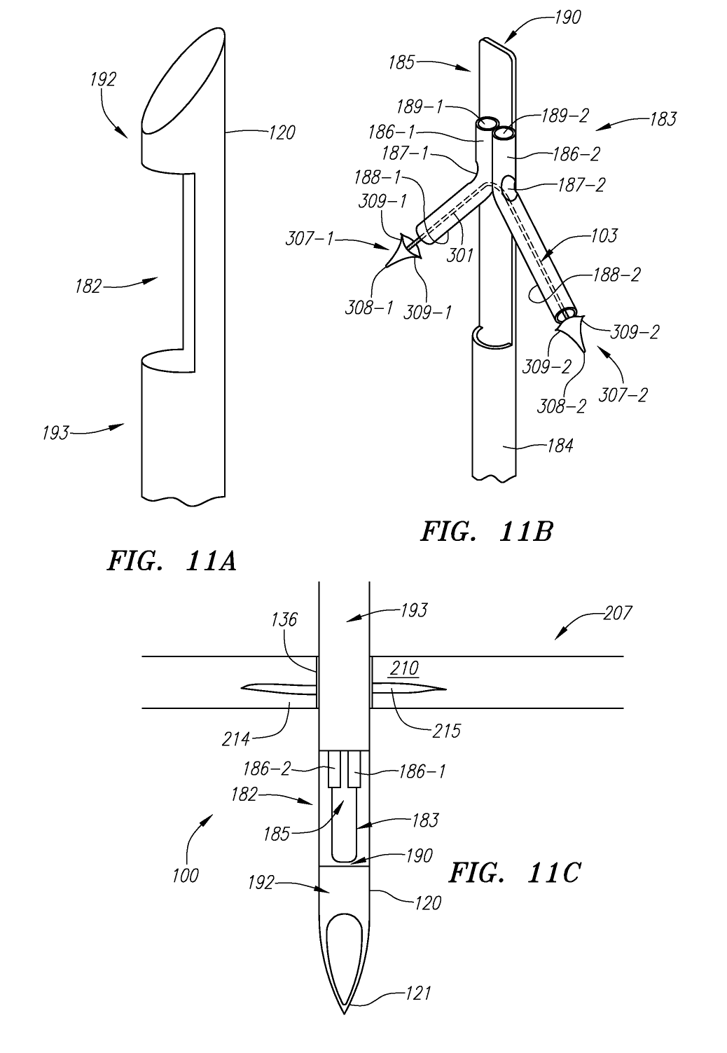

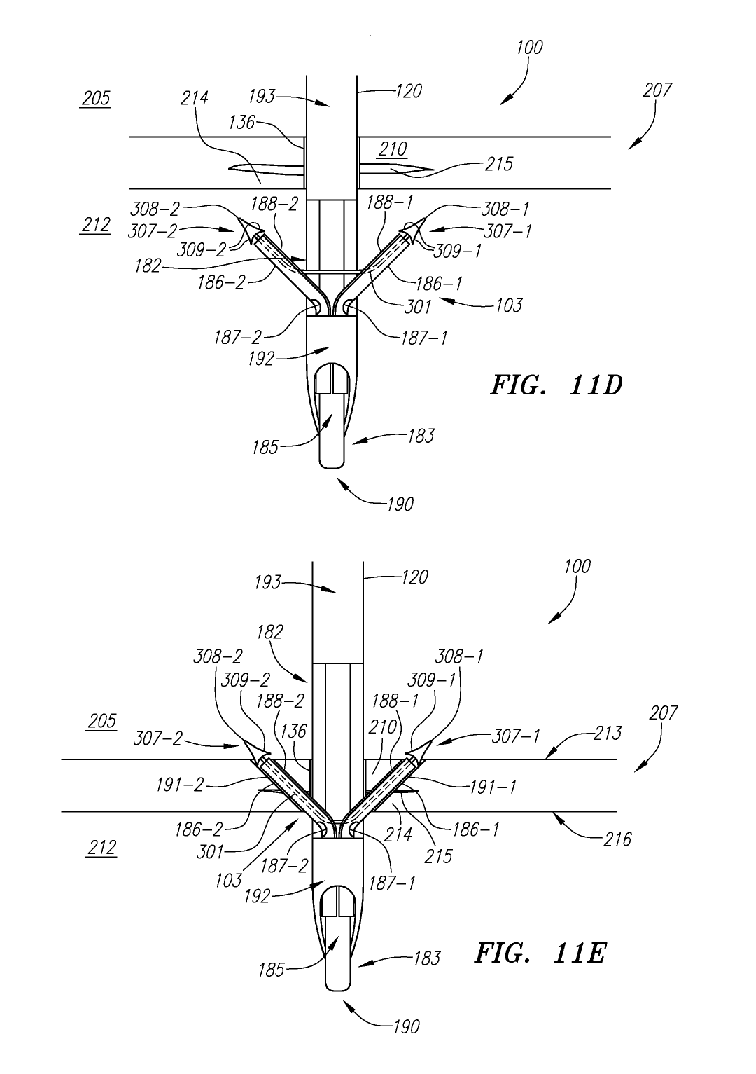

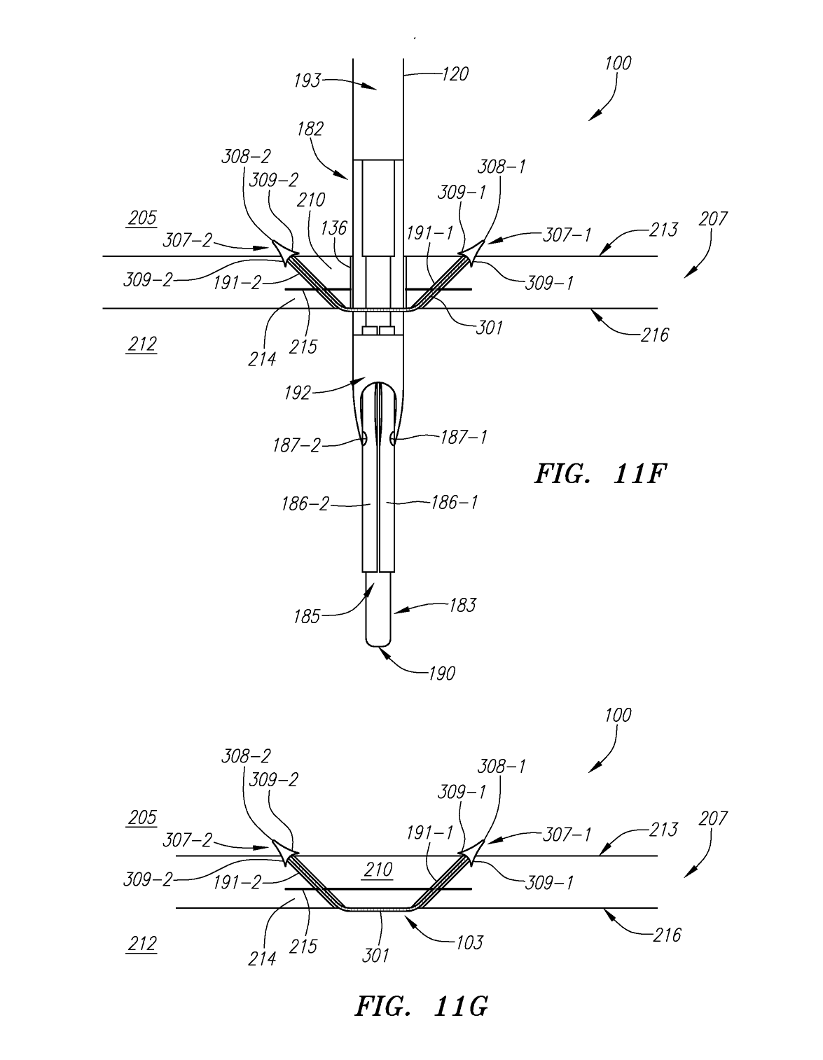

FIGS. 11A-G depict another exemplary embodiment of system 100. In this embodiment, system 100 is configured to treat the PFO with a suture 103 having ends 307. FIG. 11A is a perspective view depicting an exemplary embodiment of needle 120 configured for use with this embodiment of suture 103. Needle 120 preferably has a partially open section 182 to allow for delivery of suture 103. Partially open section 182 is preferably located between a distal tubular section 192 and a proximal tubular section 193. Needle lumen 122 is preferably configured to slidably receive a suture deployment member 183, which is depicted in the perspective view of FIG. 1B.

Suture deployment member 183, in this embodiment, has a tubular body 184 with a partially open distal section 185 and a distal end 190. Two deflectable tubular guide members 186-1 and 186-2 are coupled with body 184 in open distal section 185. Each guide member 186-1 and 186-2 is preferably biased towards the elbowed configuration depicted here, and is deflectable to a relatively straightened configuration allowing member 183 to slide within needle lumen 122. Guide members 186 can be configured with an deflection facilitating region 187, which in this embodiment is an aperture located on the inside of the elbow portion. Guide members 186 are preferably configured to house suture 103 and guide the insertion of suture 103 through septal wall 207. Each guide member 186 can include an elongate slit-like opening 188 to allow the release of suture 103 from within lumens 189 of guide members 186. This opening can also allow suture 103 to bridge between lumens 189.

FIG. 11C is a partial cross-sectional view depicting this embodiment during a treatment procedure. Here, needle 120 has been positioned as desired (preferably with OA delivery device 104, which is not shown) and advanced through septal wall 207 to create opening 136. Suture deployment member 183 can be held in position with respect to needle 120 such that guide members 186 remain in the relatively straightened configuration within proximal tubular section 193.

In FIG. 11D, suture deployment member 183 has been advanced distally to allow guide members 186 to deflect outward from needle open section 182. This deflection can cause suture 103 to slide further outside of guide members 186 through slit 188 as depicted, although suture 103 can be configured to remain within guide members 186 (e.g., through use of a stretchable suture body 301 or by using a relatively longer suture body 301 and the like). Once in the deflected configuration shown here, suture deployment member 183 can be retracted proximally (either with needle 120 or with respect to needle 120 and septal wall 207) to insert substantially sharp, needle-like proximal tips 308 into septal wall 207.

FIG. 11E depicts system 100 after suture deployment member 183 and needle 120 have been proximally retracted together by a desired amount. Proximal tips 307 and guide members 186-1 and 186-2 have created openings 191-1 and 191-2, respectively, in septal wall 207. In this embodiment, as suture ends 307 are inserted into septal wall 207, the relative angle of deflection of guide members 186 with respect to needle 120 increases, i.e., guide members 186 deflect outwards as they are inserted through the septal tissue. It should be noted that suture ends 307 can be inserted into septal wall 207 (either or both of secundum 210 and primum 214) by any desired amount, preferably sufficient to allow one or more retainment elements 309 to anchor within the septal tissue. In this embodiment, retainment elements 309 are distally located barbs, although other elements can be used. Also, as shown here, ends 307 have been advanced entirely so as to penetrate the opposite surface 213 of septal wall 207, although this is not required.

After penetration of septal wall 207 is complete, suture deployment member 183 can be distally advanced (with needle 120 or with respect to needle 120 and septal wall 207) to withdraw guide members 186 from septal wall 207. Retainment elements 309 act to retain suture 103 within septal wall 207, pulling the remainder of suture body 301 from guide members 186 through slits 188. FIG. 11F depicts system 100 after suture deployment member 183 has been advanced distally causing guide members 186 to deflect back into a relatively straightened state where they are maintained within distal tubular section 192. Deflection facilitation regions 187 are preferably oriented such that they do not impede the advancement of member 183 into tubular section 192 (e.g., apertures 187 are oriented so as not to catch on the edge of distal tubular section 192). At this point, needle 120 and suture deployment member 183 can be proximally retracted from septal wall 207, leaving suture 103 implanted within septal wall 207 as depicted in FIG. 11G.

The central portion of suture 103 not located within openings 191-1 and 191-2 is routed over the surface 216 of primum 214. Preferably, suture body 301 is sized small enough such that implantation in this configuration is sufficient to at least partially, and preferably fully, close PFO tunnel 215. Alternatively, suture body 301 can be elastic, spring-like and the like and configured to self-adjust the body length to close tunnel 215. In yet another embodiment, suture body 301 can be adjusted or tightened prior to withdrawal of needle 120, using other techniques as desired.

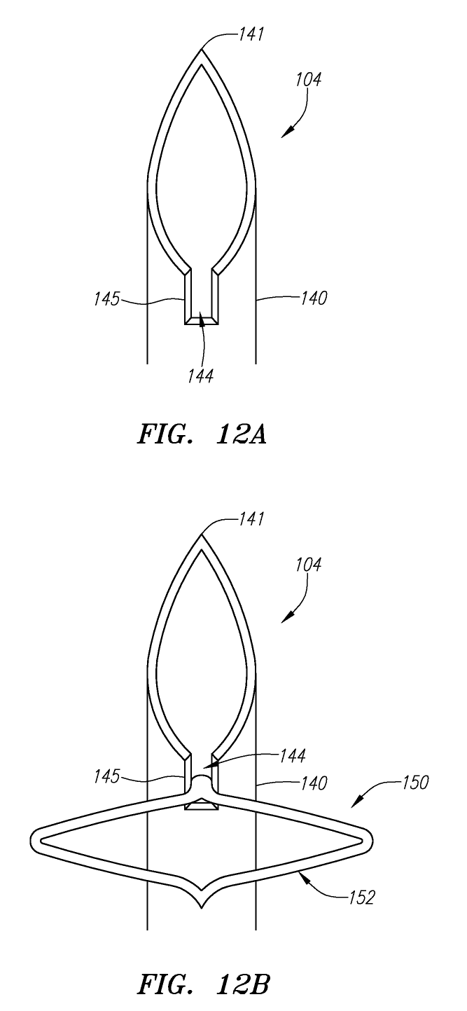

Turning now to members 120 and 140, each can be configured in any manner desired in order to facilitate deployment of snare 150. FIG. 12A is a perspective view depicting an exemplary embodiment of needle 140 having a slot 144 configured to aid in the orientation of snare 150. Slot 144 is preferably located in the proximal portion of distal end 141. The sidewalls 145 of slot 144 are preferably dull to reduce the risk of damaging snare body 151 or suture 103. Slot 144 is preferably configured to receive snare 150 (not shown) after snare 150 is deployed and snare head portion 152 folds back. Due to the angled construction of needle distal end 141, retraction of snare 150 will cause snare body 151 to slide proximally into slot 144 as depicted in FIG. 12B. The use of slot 144 can allow automatic orientation of snare head portion 152 with respect to needle 140.

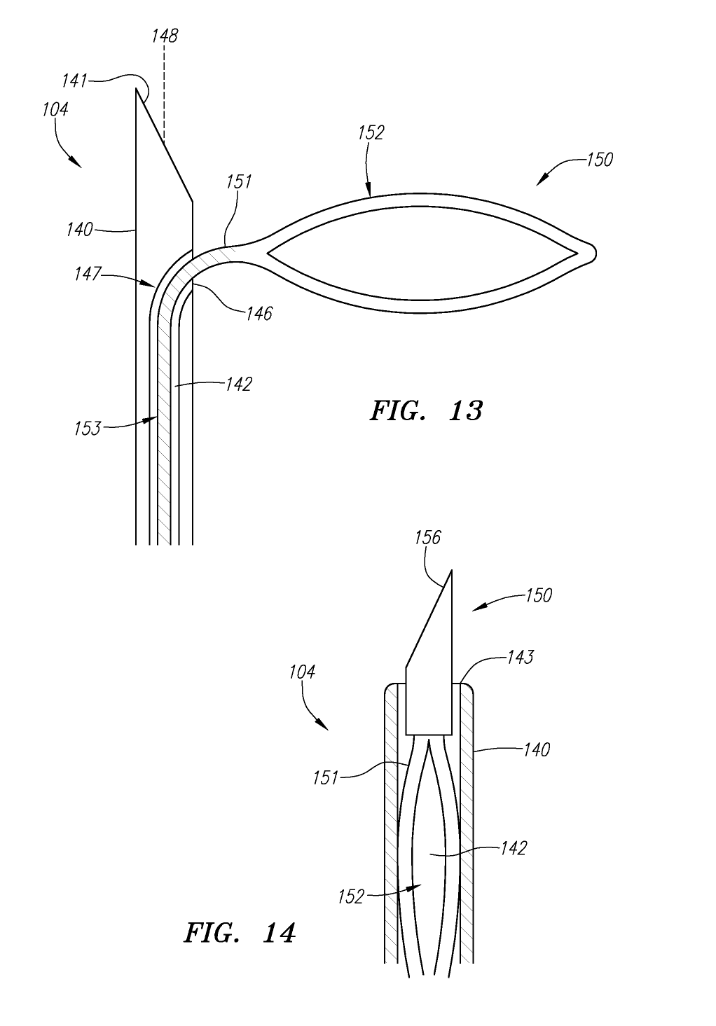

FIG. 13 depicts another exemplary embodiment of needle 140 configured to facilitate deployment of snare 150. Here, needle 140 has an inner lumen 142 with a curved portion 147 that ends in open side port 146. Snare 150 is configured to slide within lumen 142 and deploy from side port 146. Due to curved portion 147, snare 150 is deployed at an angle with respect to central axis 148 of needle 140. This facilitates the orientation of snare head portion 152 with respect to septal wall 207 (not shown) and also allows for relatively easier retraction of snare portion 150. Because snare 150 is deployed from side port 146, substantially sharp distal end 141 of needle 140 can be configured in a solid, trocar-like manner.

In one exemplary embodiment, needle distal end 141 is not required to be substantially sharp, with the tissue penetrating surface instead being distal end 156 of snare 150. FIG. 14 is a cross-sectional view depicting an exemplary embodiment where snare distal end 156 is substantially sharp and configured to penetrate septal wall 207 (not shown). To penetrate the desired portion of septal wall 207, snare 150 is preferably maintained in a position within inner lumen 142 such that snare distal end 156 extends past relatively dull distal end 143 of member 140, as depicted here. Snare 150 and member 140, when kept in this position, can together be advanced into and through septal wall 207. Alternatively, snare 150, alone, can be advanced through septal wall 207 and member 140 can then be advanced through the opening created by snare 150. It should be noted that in any of the embodiments described herein, instead of using needles 120 and/or 140, the tissue piercing structure can instead be placed on the distal end of closure element 103, as well as capture devices 150 and/or 164. Also, OA delivery members 401-1 and/or 401-2 can be configured with sharp distal ends to create the openings in septal wall 207 instead of needle 120 and/or 140.

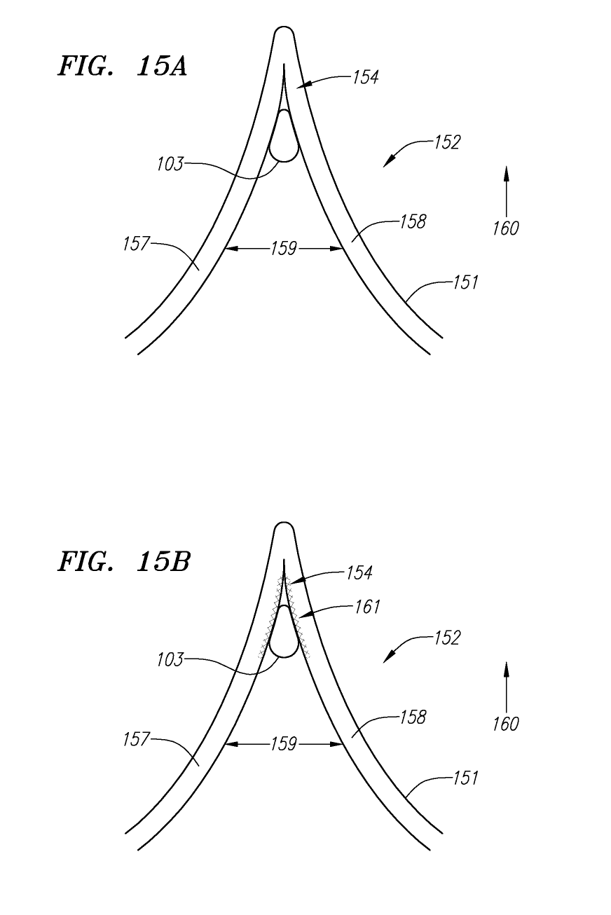

In addition to the configuration depicted in FIG. 14, snare 150 can also be configured in any other manner desired to facilitate capture and retrieval of suture 103. FIGS. 15A-B are top down views depicting exemplary embodiments of snare head portion 152. In the embodiment depicted in FIG. 15A, wedge-shaped catch portion 154 is shown in detail. Here, it can be seen that catch portion 154 is formed by a convergence of the left and right sides of snare body 151, here referenced as sides 157 and 158, respectively. Sides 157 and 158 converge at a mainly decreasing rate. In other words, the distance 159 between sides 157 and 158 decreases as one views portion 152 at different successive positions in direction 160, i.e., the distal direction, but the amount distance 159 decreases at each successive position in direction 160 is less than the previous position. This increases the tendency of suture 103 to become caught and secured within catch portion 154.

FIG. 15B depicts another exemplary embodiment of snare head portion where the surface 161 of snare body 151 within catch portion 154 is textured to increase the surface friction between body 151 and suture 103, in order to more easily catch and trap suture 103. It should be noted that surface 161 can be textured in any manner desired, such as by etching, abrading, or coating body 151 and the like.

FIGS. 16-19C depict additional exemplary embodiments of system 100 where snare 150 and/or suture 103 are configured to further increase the capture ability. FIG. 16 is a top down view depicting an exemplary embodiment of snare 150 where body 151 in snare head portion 152 is coiled, with the exception of catch portion 154. Specifically, body 151 is configured as a metal microcoil that can be more likely to snare, grasp, catch or engage suture 103 at any angle of orientation. Retraction of snare 150 back into member 140 can cause the coils to compress and further secure the grasp on suture 103, even if suture 103 does not become trapped in catch portion 154. In another exemplary embodiment, catch portion 154 can also be coiled.

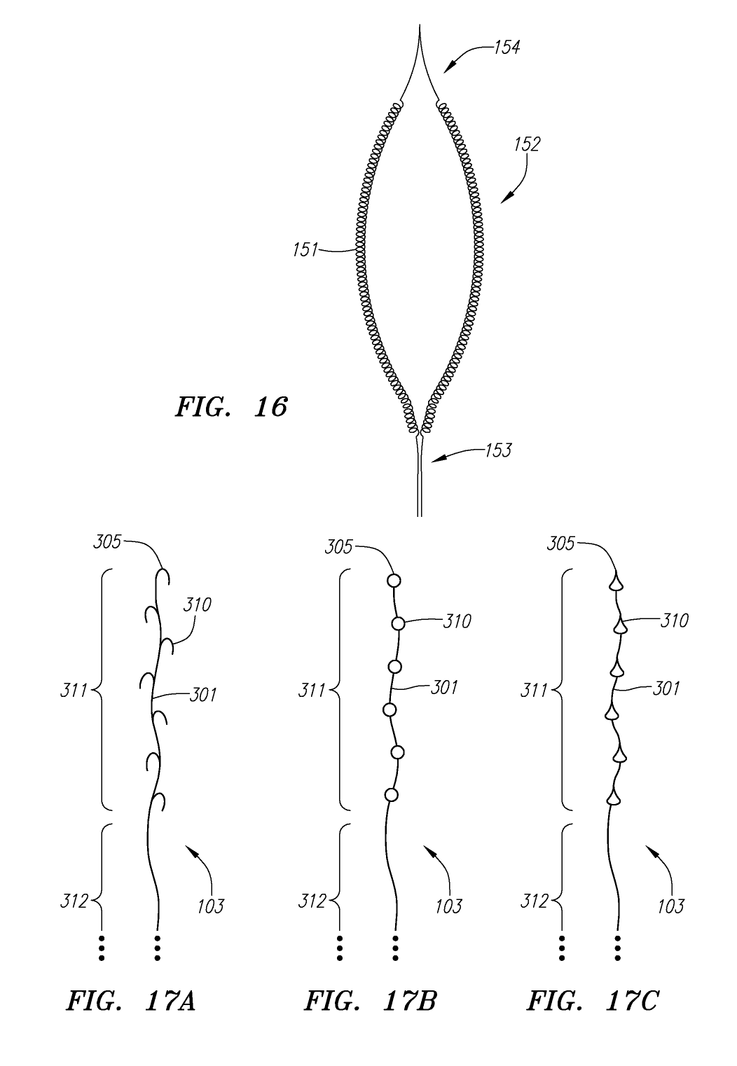

FIGS. 17A-C depict exemplary embodiments of suture 103 configured for use with any of the embodiments of snare 150 described herein. FIG. 17A is a top down view depicting an exemplary embodiment of suture 103 having protrusions 310 extending from a distal portion 311 of body 301. Here, each of these protrusions 310 is in a curved configuration, somewhat like a “fish-hook.” Preferably, protrusions 310 can be configured with a degree of flexibility that allows protrusions 310 to deflect to avoid catching the tip of needle 140 when being pulled into needle 140 via snare 150. FIGS. 17B-C depict additional exemplary embodiments where protrusions 310 have solid bead-like and cone-like configurations, respectively. Protrusions 310 provide additional surface area for snare 150 to engage and grasp, and subsequently facilitate the capture and retrieval processes. In these embodiments, protrusions 310 are only located on distal portion 311. Proximal portion 312 does not include protrusions 311 to minimize contact with the septal wall tissue during and after implantation. It should be noted that protrusions 310 can be present along any portion of body 301 and can be configured in any manner desired, with any shape and size, and are not limited to the configurations described with respect to FIGS. 17A-C.

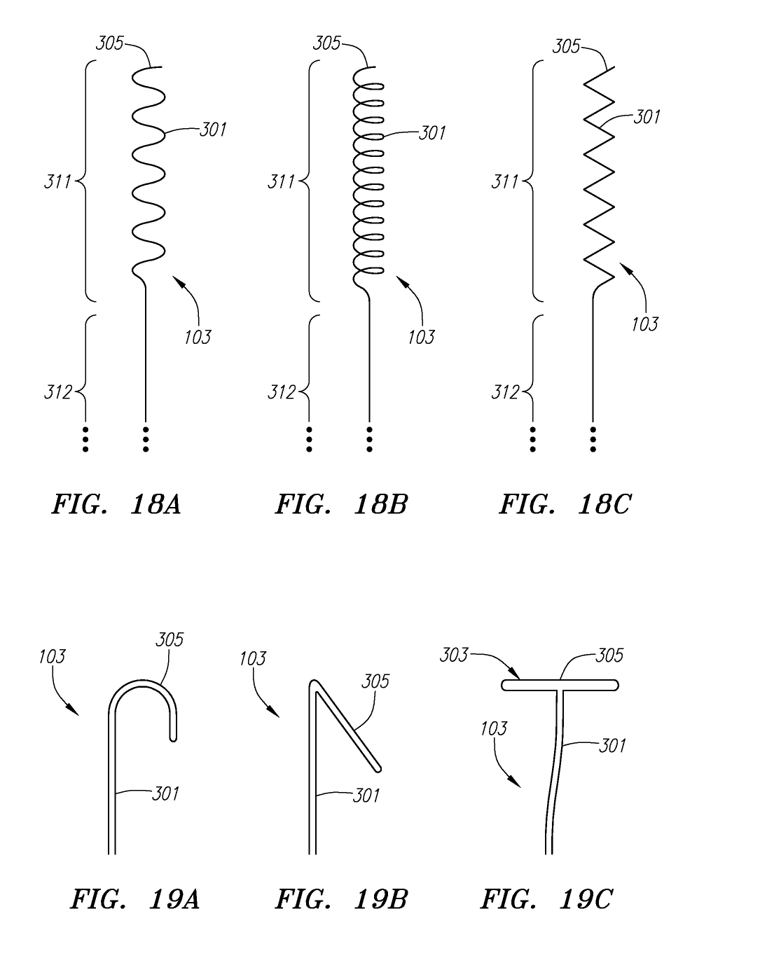

FIG. 18A is a top down view depicting another exemplary embodiment of suture 103. Here, distal portion 311 of suture 103 has a curved, serpentine-like configuration for facilitating the capture and retrieval process. FIGS. 18B-C depict additional exemplary embodiments where suture 103 has a helical coiled configuration and jagged sawtooth configuration, respectively, each of which can also facilitate the capture and retrieval process. It should be noted that the distal portions 311 of each of the embodiments depicted in FIGS. 18A-C can also be curved.

FIGS. 19A-C are top down views depicting additional exemplary embodiments of suture 103. Here, distal end 305 of suture 103 is curved (as depicted in FIG. 19A) and bent (as depicted in FIG. 19B) to facilitate capture. Also, as will be discussed below, suture distal end 305, in addition to suture proximal end 304, can also include anchor device 303. Anchor device 303 can be configured to facilitate capture, like the “T” type anchor device 303 depicted in FIG. 19C. It should be noted that the configurations of distal end 305 described with respect to FIGS. 19A-B can also be implemented as anchor device 303.

As discussed above, in one exemplary embodiment of system 100, proximal end 304 of the implanted suture 103 can be anchored against septal wall 207 with anchor device 303 and distal end 305 can be anchored against septal wall 207 with lock device 302. However, system 100 is not limited to implanting suture 103 in this manner and, in fact, suture 103 can be anchored in any fashion desired. For instance, FIG. 20A is a partial cross-sectional view depicting an exemplary embodiment where both distal end 305 and proximal end 304 are anchored against septal wall 207 with an anchor device 303. The use of two anchor devices 303 generally requires that the length of suture 103 be taken into account to ensure that suture 103 is not too long to adequately close PFO tunnel 215. Alternatively, the length of suture body 301 can be variable as in the instances where body 301 is elastomeric or spring-like.

FIG. 20B depicts another exemplary embodiment where both distal end 305 and proximal end 304 are anchored against septal wall 207 with a separate lock device 302. Lock devices 302 can each be deployed from needles 120 and 140. FIG. 20C depicts another exemplary embodiment where distal end 305 and proximal end 304 are each anchored against septal wall 207 with the same lock device 302. Lock device 302 can be deployed from body member 101 (not shown) over needles 120 and 140 (not shown) into the position depicted here. It should be noted that the embodiments depicted in FIGS. 20A-C are merely examples not intended to limit the manners in which suture 103 can be implanted.

Anchor device 303 and lock device 302 can be implemented in numerous different configurations. A non-exhaustive list of the many different embodiments of anchor device 303, including “T” anchors, and lock device 302 are discussed in detail in the incorporated application entitled “Suture-based Systems and Methods for Treating Septal Defects” (Ser. No. 11/218,794).

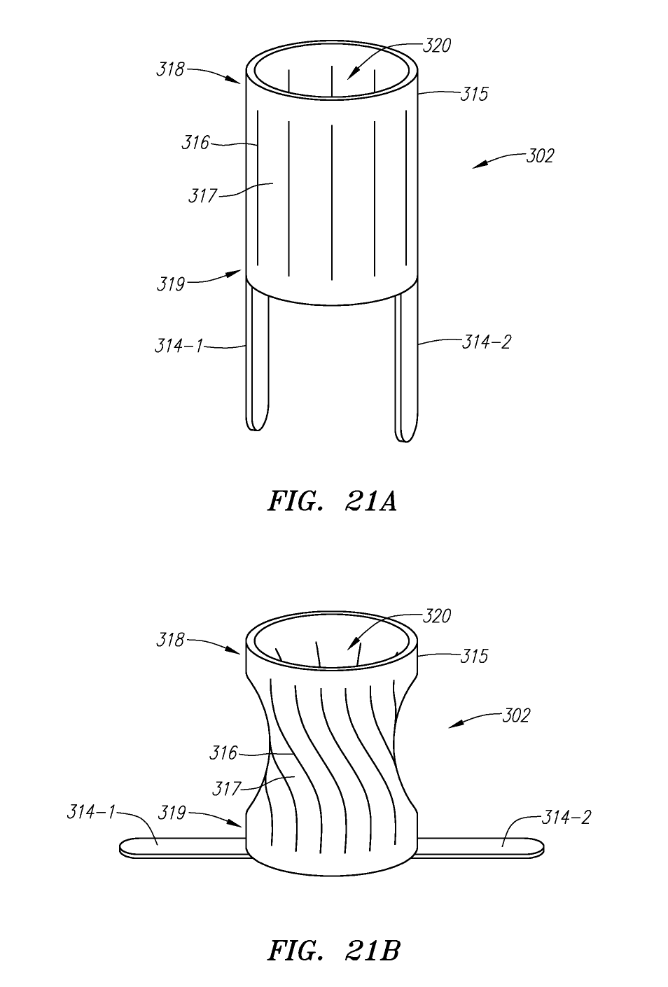

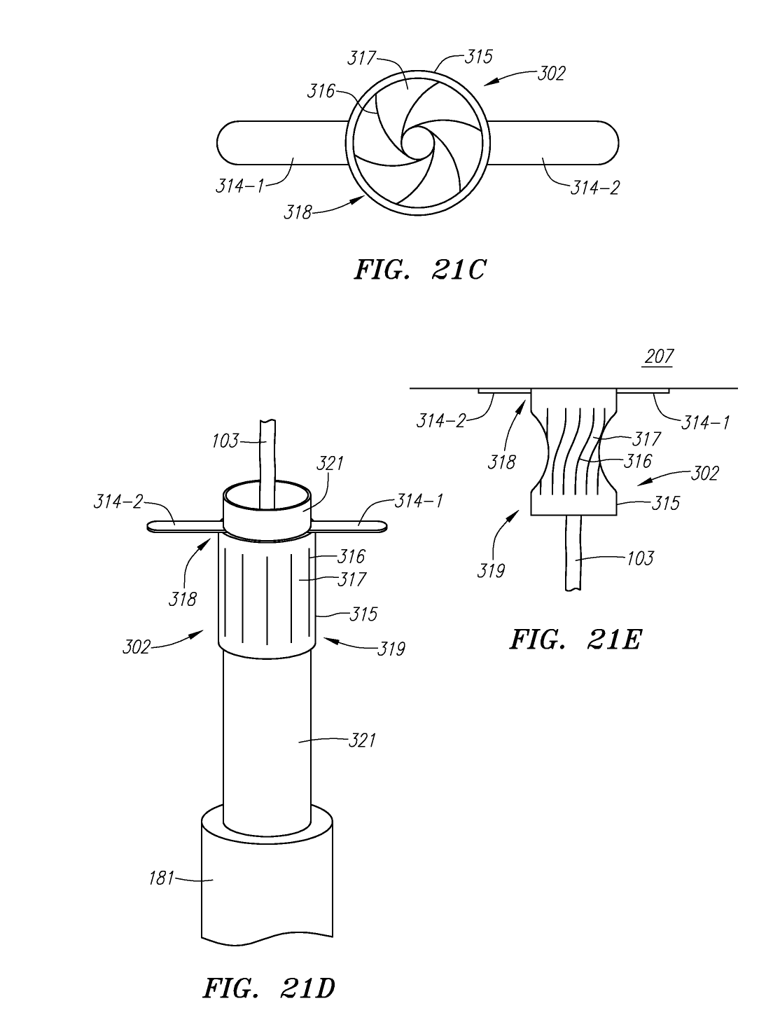

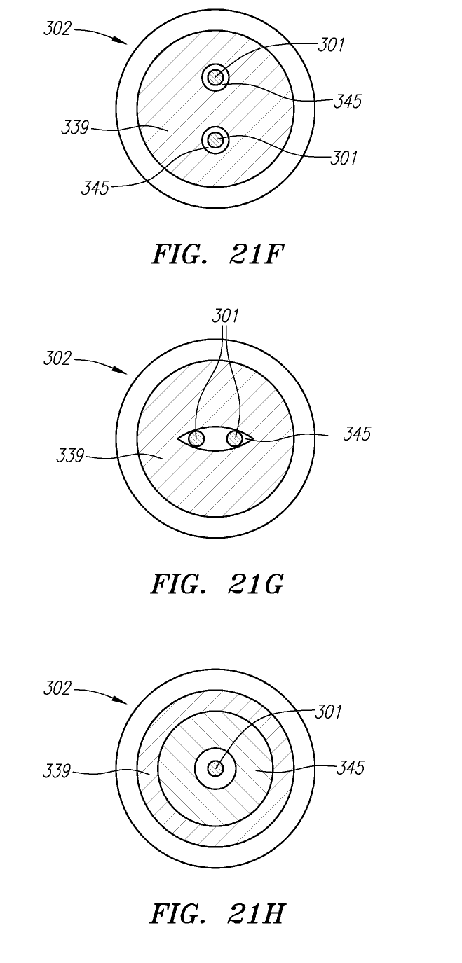

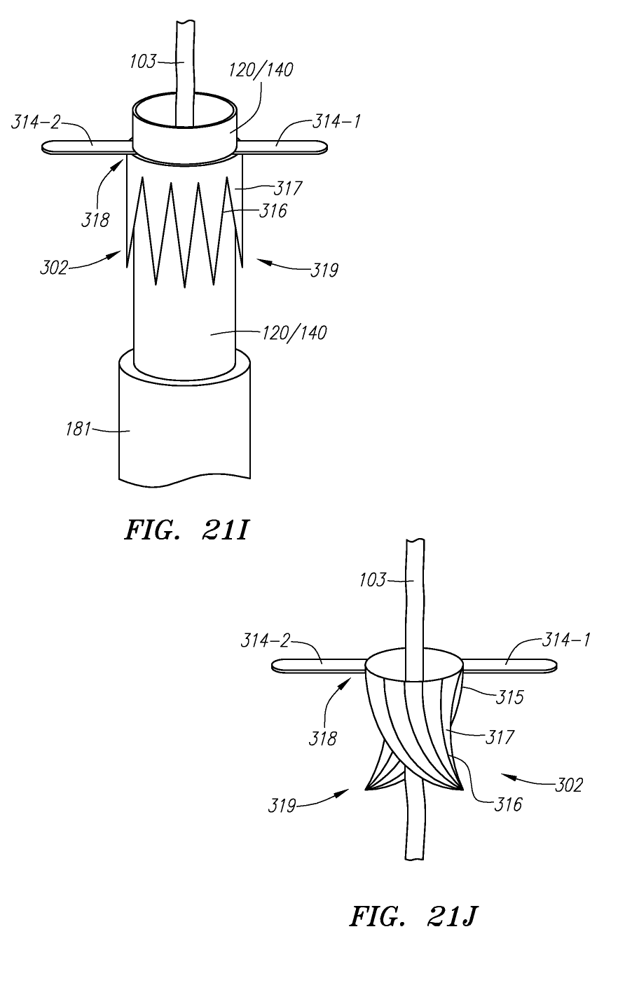

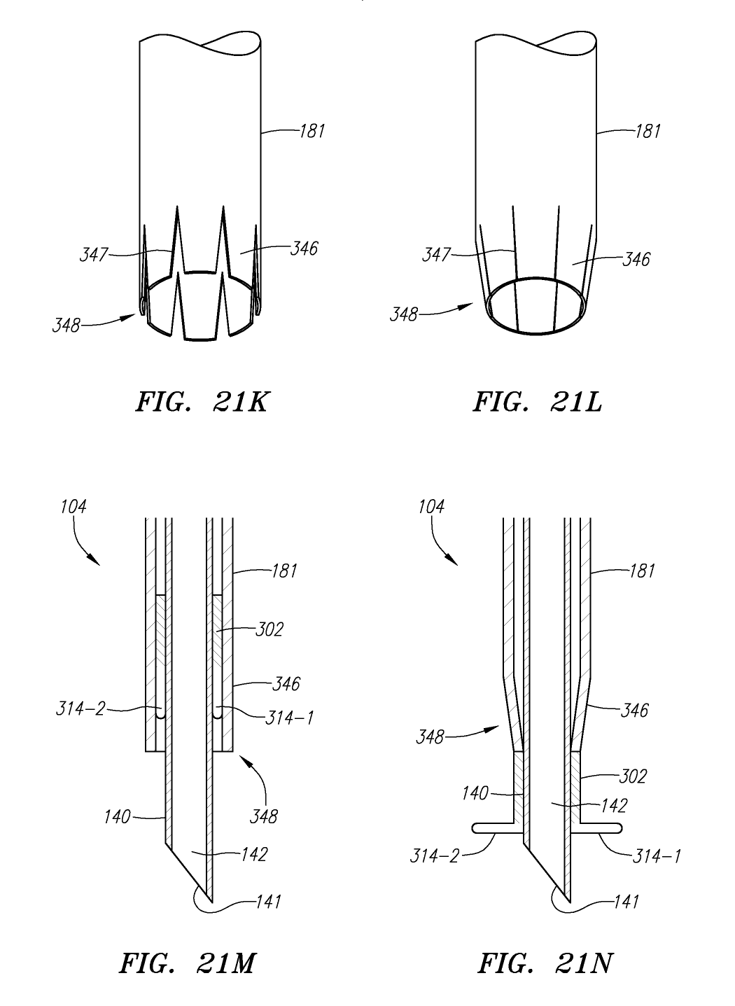

FIGS. 21A-25C depict additional exemplary embodiments of various lock devices that can be used with the embodiments described herein in addition to those known to persons of ordinary skill in the art. These embodiments can be used in any type of application and for any purpose both within and outside of the medical field. These embodiments can be used with the other systems and methods described herein to provide a lock on suture 103 to prevent movement of suture 103 through tissue, or to lock any number sutures 103 together. These embodiments can be fabricated in any desired manner, including, but not limited to, laser cutting, etching, or machining tube or wire stock, or laser cutting, etching, or machining sheets and then rolling or wrapping the sheets into the desired configuration. Other elements such as cutting elements and the like or features such as textured surfaces and the like can be added via separate steps or procedures.

FIG. 21A is a perspective view depicting an exemplary embodiment of lock device 302. Here, lock device 302 includes two anchoring legs 314-1 and 314-2 and a tubular body 315 having a plurality of elongate openings 316, which define struts 317. Elongate openings 316 are not required to be relatively straight, as depicted here, and can assume other patterns.

This and other embodiments of lock device 302 are preferably formed from NITINOL or some other elastic (e.g., steel) or superelastic material. Lock device 302 can also be configured with temperature-based shape memory characteristics. Lock device 302 is preferably heat treated in the configuration depicted in FIG. 21B, where a first end 318 has been rotated with respect to a second end 319 to cause struts 317 to bend inwards into lumen 320 of body 315, as depicted in the top down view of FIG. 21C. Anchoring legs 314 are also preferably in an outwardly deflected position to anchor lock device 302 against the desired tissue surface. Anchoring legs 314 can be deflected back to the configuration of FIG. 21A to allow lock device 302 to fit within a tubular member, such as a needle, OA member and the like. As an alternative to heat treatment, lock device 302 can be configured to use the temperature sensitive shape memory characteristics of NITINOL to effect the transition from the unlocked to the locked configuration.

Deployment of this and other embodiments of lock device 302 can be achieved with use of a restraining member 321 and a pusher member 181 as depicted in FIG. 21D. Here, restraining member 321 is a tubular member having a lumen through which suture body 301 can be routed (instead of a separate restraining member 321, use of members 120 or 140 can be substituted). Pusher member 181 can also be a tubular member configured to slidably receive restraining member 321. Pusher member 181 can be used to distally advance lock device 302 off of restraining member 321 to allow lock device 302 to return to the twisted configuration. Upon doing so, struts 317 preferably engage suture 103 and prevent lock device 302 from moving with respect to suture 103. Lock device 302 in the locked configuration on suture 103 against septal wall 207 is depicted in FIG. 21E. To increase the surface friction between lock device 302 and suture 103, struts 217 can be configured with a roughened or textured surface and the like. It should be noted that instead of configuring lock device 302 to twist as described here, lock device 302 can also be configured such that struts 317 deflect inwards towards a central longitudinal axis of the device 302 without twisting.

Lock device 302 can be further configured with a compliant material 339 disposed within lumen 320. FIGS. 21F-H are top down views depicting additional exemplary embodiments of lock device 302 with compliant material 339 in lumen 320. Lining 339 can have any thickness desired. In FIG. 21F, lumen 302 is preferably entirely filled with material 339 such that two separate, relatively smaller inner lumens 345 can be formed therein. In FIG. 21G, material 339 lines the surface of lumen 302 to form an elliptical inner lumen 345 in which two suture bodies are placed, while in FIG. 21H, material 339 lines the interior of lumen 302 to form a circular inner lumen 345. Compliant material 339 can be any material desired, preferably one that provides increased surface friction with suture body 301. Examples include, but are not limited to silicone, polyurethane, polyether block amides, hydrogel elastomers and/or hydrophilic polymers. Material 339 can be applied in any manner desired, including, but not limited to applying the material directly to lumen 320 or bonding the material in tubular form to the surface of lumen 320.

FIGS. 21I-J are perspective views depicting another exemplary embodiment of lock device 302 similar to the one described with respect to FIGS. 21A-H. Here, struts 317 are unconnected at end 319 so that each strut 317 is movable independent of the others. Openings 316 are tapered to give struts 317 a crown-like appearance. FIG. 21I depicts this embodiment prior to deployment from restraining member 321 with pusher member 181, while FIG. 21J depicts this embodiment engaged with suture 103 after removal of restraining member 321. The ends of each strut 317 can be relatively sharp as depicted here or relatively dull in other embodiments.

FIGS. 21K-N depict embodiments for advancing lock device 302 off of the member on which lock device 302 resides (e.g., needles 120/140, member 321, etc.), preferably for embodiments where lock device 302 includes one or more anchoring legs 314. FIG. 21K is a perspective view depicting an exemplary embodiment of pusher member 181, where the distal end region 348 is configured with multiple tapered struts 346 separated by slots 347. Preferably, struts 346 are biased to deflect to the configuration depicted in FIG. 21L. In one exemplary embodiment, to achieve this bias, pusher member 181 can be composed of NITINOL and heat-treated in this configuration of FIG. 21L.

FIGS. 21M-N are partial cross-sectional views depicting delivery device 104 with the embodiment of pusher member 181 described with respect to FIGS. 21K-L. In FIG. 21M, pusher member 181 is shown in the relatively undeflected state, with struts 346 positioned over lock device 302 and needle 140 such that lock device 302 keeps struts 346 from deflecting inwards. In this position, struts 346 restrain anchoring legs 314 from deflecting outwards. When the user is ready to deploy lock device 302, pusher member 181 can be proximally retracted to position distal end region 348 proximal to lock device 302, as depicted in FIG. 21N. Anchoring legs 314 are free to deflect outwards once distal end region 348 is moved proximal to legs 314, and once distal end region is proximal to lock device 302, struts 346 are free to deflect inwards against needle 140. In this configuration, pusher member 181 can abut lock device 302 and be used to distally advance lock device 302 from needle 140.

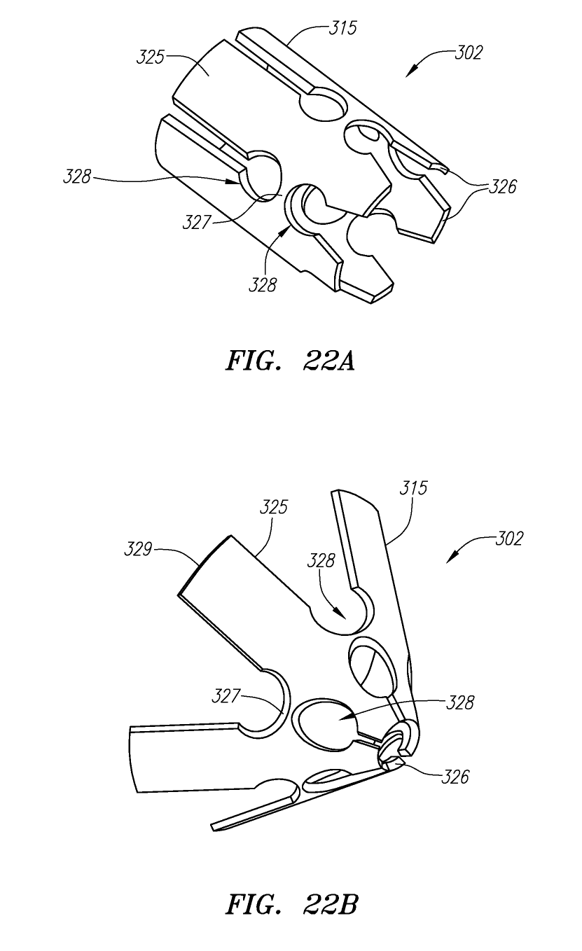

FIGS. 22A-B are perspective views depicting an exemplary embodiment of a “cone-like” lock device 302 in the undeployed and deployed configurations, respectively (restraining member 321, pusher member 181 and suture 103 are not shown). In this embodiment, lock device 302 includes five concentrically arranged lever arms 325 configured to pivot from the tubular, unlocked configuration of FIG. 22A to the cone-like, locked configuration of FIG. 22B. Each lever arm 325 has a retaining end 326 configured to abut suture 103 and prevent lock device 302 from moving with respect to suture 103. Retaining ends 326 are preferably configured to engage suture 103 and can include barbs, textured surfaces or any other feature that may increase the amount of surface friction between each lever arm 325 and suture 103. In this embodiment, retaining ends 325 are tapered to allow a high degree of deflection into the cone-like configuration, i.e., to allow lock device 302 to deflect from the tubular configuration into a relatively more planar configuration. It should be noted that lock device can be configured to transition through the cone-like configuration into a planar configuration to maximize the locking effect.

To allow lever arms 325 to pivot, each lever arm 325 is preferably coupled together via a hinge 327. In this embodiment, hinge 327 has a living hinge-type configuration and is formed by a relatively thin portion of the lock device body 315 disposed between adjacent rounded apertures 328. The relatively thin portion 327 is configured to flex between the unlocked and locked configurations, as depicted here. It should be noted that any type of hinge 327 can be used. For instance, in one exemplary embodiment hinge 327 is a mechanical hinge (e.g., a ball and socket hinge, a swivel hinge and the like) and lock device 302 includes one or more bias elements (e.g., such as a spring or compressible element and the like) configured to bias lever arms 325 to transition towards the locked configuration.

When this embodiment of lock device 302 is deployed, it is preferably done so with retaining ends 326 oriented in a position away from the direction in which the most tensile force on suture 103 is expected to come. For instance, if lock device 302 is deployed on suture 103 and intended to prevent suture 103 from being pulled through septal wall 207, then ends 329 of lever arms 325, which are opposite retaining ends 326, are preferably positioned against septal wall 207. When positioned in this manner, the pulling of suture 103 will act to force lever arms 325 towards a more planar locked configuration, thereby forcing retaining ends into suture 103 and increasing the surface friction between lock device 302 and suture 103.

It should be noted that any number of lever arms 325 can be used, arranged in any fashion, symmetric or asymmetric. Furthermore, although shown in FIG. 22A with a generally cylindrical configuration with a generally circular radial cross-section, lock device 302 can have other configurations, including having radial cross-sections that are elliptical, polygonal, irregular, combinations thereof and the like. Lock device 302 described with respect to FIGS. 22A-B can be deployed with any desired device, including, but not limited to the embodiment of pusher member 181 described with respect to FIGS. 21K-N.

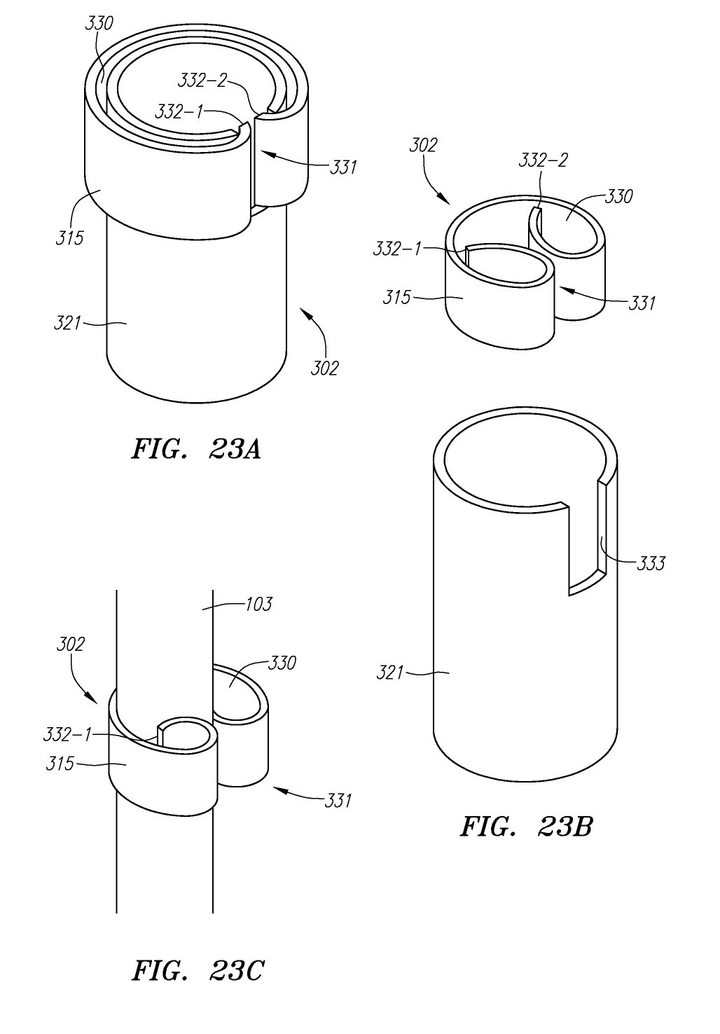

FIGS. 23A-C are perspective views depicting another exemplary embodiment of lock device 302. FIG. 23A depicts lock device 302 in the unlocked configuration on restraining member 321. Here, lock device 302 has tubular body 315 having a lumen 330. Body 315 has an elongate longitudinal opening 331 extending the length of the body, to form opposing ends 332-1 and 332-2. Lock device 302 is preferably biased towards the configuration depicted in FIG. 23B, where lock device 302 has been deployed from restraining member 321. In this configuration, body 315 is configured such that ends 332-1 and 332-2 enter lumen 330 and begin to curl or roll inwards. FIG. 23C depicts this embodiment locked over suture 103. Referring back to FIG. 23B, in this embodiment restraining member 321 has a distal notch 333 configured to receive ends 332-1 and 332-2 (if members 120 or 140 are used in place of restraining member 321, notch 333 can be placed in the distal end of that member 120/140). Also, although not shown, this embodiment can be configured with anchoring legs 314 in any desired position.

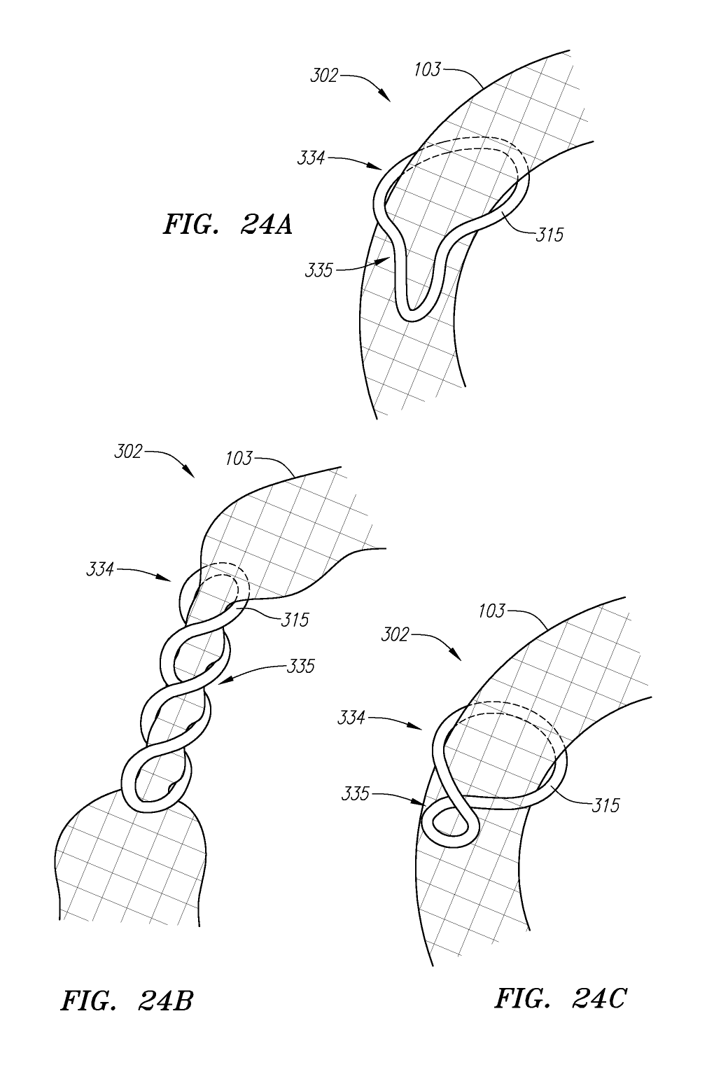

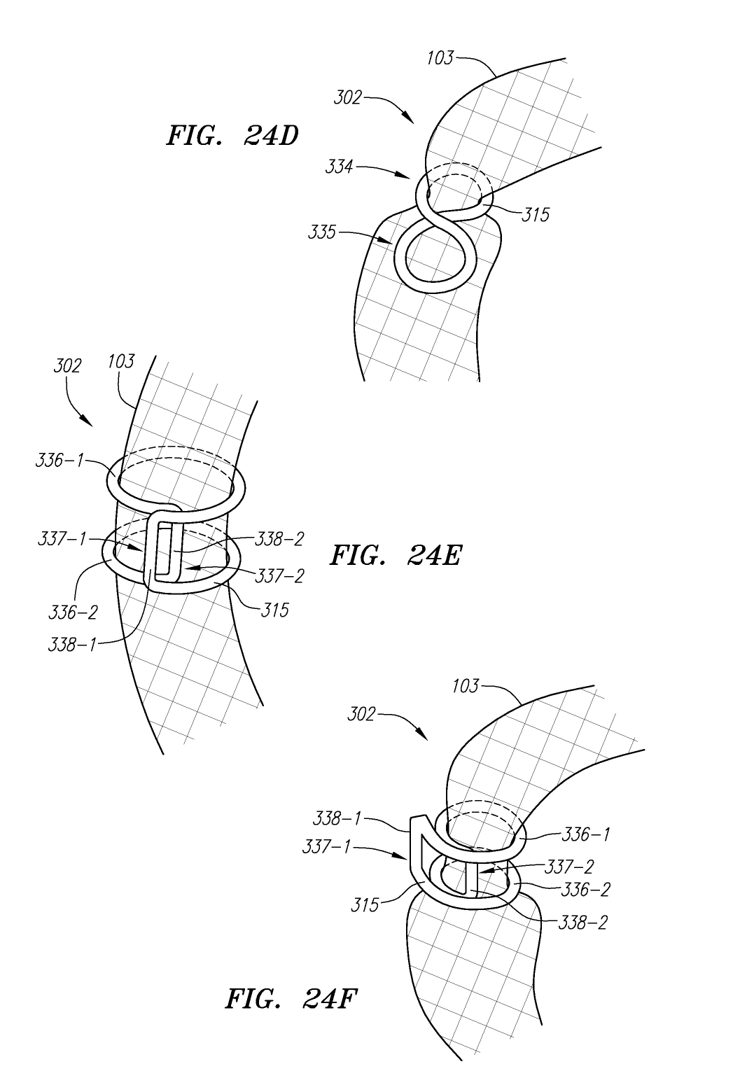

FIGS. 24A-F are perspective views depicting additional exemplary embodiments of lock device 302. In these embodiments, lock device 302 has a wire-like body 315. Body 315, although being wire-like, can be fabricated from wire, sheets or tube stock and the like. FIG. 24A depicts an exemplary embodiment of lock device 302 immediately after deployment before lock device 302 has transitioned to the locked configuration. Lock device 302 includes a main body portion 334 configured to at least partially surround suture 103 and a looped portion 335, which in this embodiment is disposed longitudinally along suture 103. Looped portion 335 is preferably configured to compress or twist to tighten main body portion 334 around suture 103, and enter the locked configuration as depicted in FIG. 24B. FIG. 24C depicts a similar exemplary embodiment of lock device 302 in the unlocked configuration, except in this embodiment, looped portion 335 is configured to expand to tighten main body portion 334 around suture 103, as depicted in the locked configuration of FIG. 24D.

FIG. 24E depicts yet another exemplary embodiment of lock device 302 in the unlocked configuration. Here, lock device 302 includes two generally annular ring portions 336-1 and 336-2 with ends 337-1 and 337-2, which can overlap in this configuration if desired. Each ring portion 336 can be connected with one or more longitudinal struts 338-1 and 338-2. Although only two ring portions 336 are shown, it should be noted that any number of ring portions 336 can be used. Furthermore, struts 338-1 and 338-2 can be placed at ends 337-1 and 337-2, as shown, or in other locations along ring portions 336. FIG. 24F depicts this embodiment in the locked configuration around suture 103. Although not shown in FIGS. 24A-F, it should be noted that these embodiments can be configured with one or more anchoring legs 314 in any desired position.

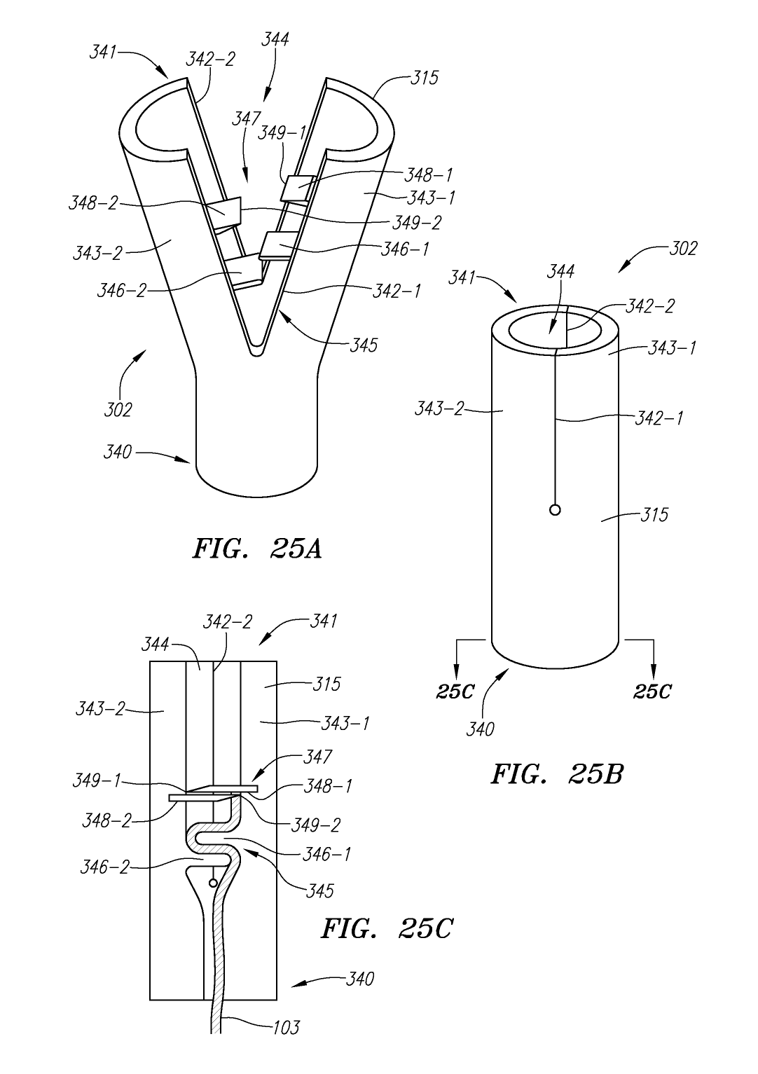

FIGS. 25A-C depict an additional exemplary embodiment of lock device 302, where lock device 302 is also configured to cut or sever one or more sutures 103. FIGS. 25A-B are perspective views of lock device 302 in the unlocked and locked configurations, respectively. Here, lock device 302 has a generally tubular body 315 with ends 340 and 341 and a lumen 344. Two elongate openings 342-1 and 342-2 extend from end 341 towards end 340 to form deflectable clamping members 343-1 and 343-2. Clamping members 343-1 and 343-2 are preferably biased towards this locked configuration.

FIG. 25C is a cross-sectional view of lock device 302 taken along line 25C-25C of FIG. 25B. Here, lock device 302 can be seen to include a locking portion 345 located adjacent to a cutting portion 348. Locking portion 345 can include abutments 346-1 and 346-2 each being formed on or coupled with clamping members 343-1 and 343-2, respectively. In this embodiment, abutments 346-1 and 346-2 are staggered (abutment 346-1 is located above abutment 346-2) and preferably configured to both compress suture 103 and create a tortuous path for suture 103, to lock suture 103 in place with respect to body 315. It should be noted that any number of one or more abutments 346 can be used and each abutment 346 can be textured or otherwise configured with features that are intended to increase the surface friction with suture 103. Abutments 346-1 and 346-2 can also be placed in a non-staggered, in-line configuration such that suture 103 is compressed between the ends of abutments 346-1 and 346-2.

Cutting portion 347 is located adjacent to locking portion 345. Here, cutting portion 347 includes two cutting elements 348-1 and 348-2 formed on, or coupled with clamping members 343-1 and 343-2, respectively. Cutting elements 348 are preferably blade-like elements with substantially sharp edges 349-1 and 349-2, and are configured to cut or sever suture 103 when clamping members 343 transition from the unlocked to the locked configuration.

Although any number of one or more cutting elements 348 can be used, it is preferably to use multiple cutting elements 348 to decrease the risk of suture 103 not being fully cut. Also, cutting elements 348 can be arranged in any desired fashion. Here, cutting elements 348-1 and 348-2 are placed on opposite clamping members 343-1 and 343-2 and in close proximity to each other to apply or approximate a “shearing stress” to suture 103. Cutting elements 348 can be part of body 315 and formed from the same material, or can be formed from a separate material and coupled with body 315, e.g., such as stainless steel razor-like elements 348 coupled with a NITINOL body 315.

Suture body 301 can be formed from any desired material, including, but not limited to, metallic materials such as NITINOL, stainless steel, elgiloy and the like, polymeric materials such as polypropylene, polyester, silicone, polyurethanie, polyethylene terephthalate (PET), degradable materials and the like, or any combination thereof. Examples of degradable materials include, but are not limited to: polyglycolide (PGA); PGA/poly(ε-caprolactone); poly(dioxanone); PLA/PGA (10%/90%); polyglyconate (copolymer of glycolide and trimethylene carbonate (TMC)); polyhydroxybutyrate (PHB); polyhydroxyvalerate (PHV); polyorthoesters (POE); and polyanhydrides.

Suture body 301 can have a variable length (e.g., be configured to compress or expand), or remain with a relatively fixed length. Suture body 301 can also be braided if desired. In one embodiment, suture 103 includes a NITINOL inner coil surrounded by a braided PET sheath and is configured exert a continuous compressive force. The selection of an appropriate material preferably takes into account: manufacturability, cost, visibility to external and/or internal imaging devices (e.g., radiopacity, etc.), MRI compatibility, biodegradability, the use of FDA-predicate materials (known in long-term implantable, blood-contacting devices), and robust temperature performance (i.e., the ability to handle any expected manufacturing, sterilization, shipment or storage temperatures). For a suture body 301 containing polymeric materials, creeping issues, ESCR issues, and sterilization issues (e.g., gamma rays/E-beam can impact mechanical properties) can also be taken into account. For a suture body 301 containing metallic materials, the degree of non-abrasiveness with lock device 302 during and after deployment (to prevent severing or weakening suture body 301), resistance to fatigue or fracture, and resistance to corrosion can also be taken into account. Furthermore, any portion of suture 103 can be coated with any desired material as desired and any portion of suture 103 or treatment system 100 can be made visible by an internal or external imaging device (e.g., radiopaque, etc).

Suture body 301 can also be configured with the desired degree of biocompatibility. Criteria that can be taken into account with regards to biocompatibility include the effect of the material/design on the healing response, the potential of a material or design to cause thrombus formation or an embolic event, and the speed of the healing response (e.g., distance new tissue must migrate across to encapsulate an implant).

The surface of suture body 301 can also be configured as desired. For instance, suture body 301 can be smooth or textured based on the desired amount of surface friction. Suture body 301 can be coated, for instance, to effect surface friction or to elute drugs to promote a healing response and the like.

Suture body 301 and snare body 151 can each be configured to magnetically attract (or repel) the other. For instance, each of bodies 301 and 151 can be magnetic, or one of bodies 301 and 151 can be magnetic and the other composed of a ferro-magnetic or other magnetic material attractable to the magnetic body. Also, bodies 301 and 151 can be configured to react to the presence of an internally or externally applied magnetic field. The magnetic field is preferably applied in such a manner that suture 103 is guided through snare head portion 152. In one exemplary embodiment, distal end 305 is metallic and the presence of the applied magnetic field pulls distal end 305 in the direction of snare head portion 152.

To facilitate visualization during the closure procedure, any portion of system 100 can be made visible by an external or internal imaging device. For instance, in one embodiment radiopaque markings are added to snare distal end 156 and suture distal end 305 to make the capture process viewable via fluoroscopy, while in another embodiment an echolucent coating is added so that distal ends 156 and 305 are viewable with ultrasound devices. Suture 103 can be configured for use with any internal or external imaging device such as magnetic-resonance imaging (MRI) devices, computerized axial tomography (CAT) scan devices, X-ray devices, fluoroscopic devices, ultrasound devices and the like.

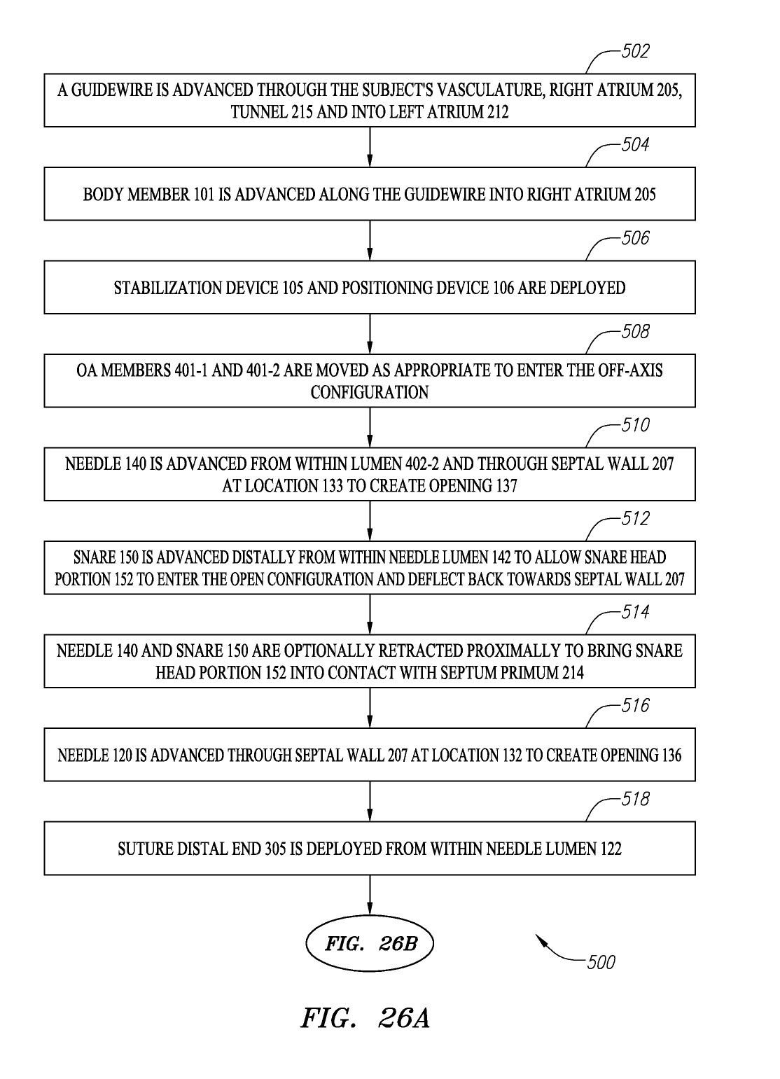

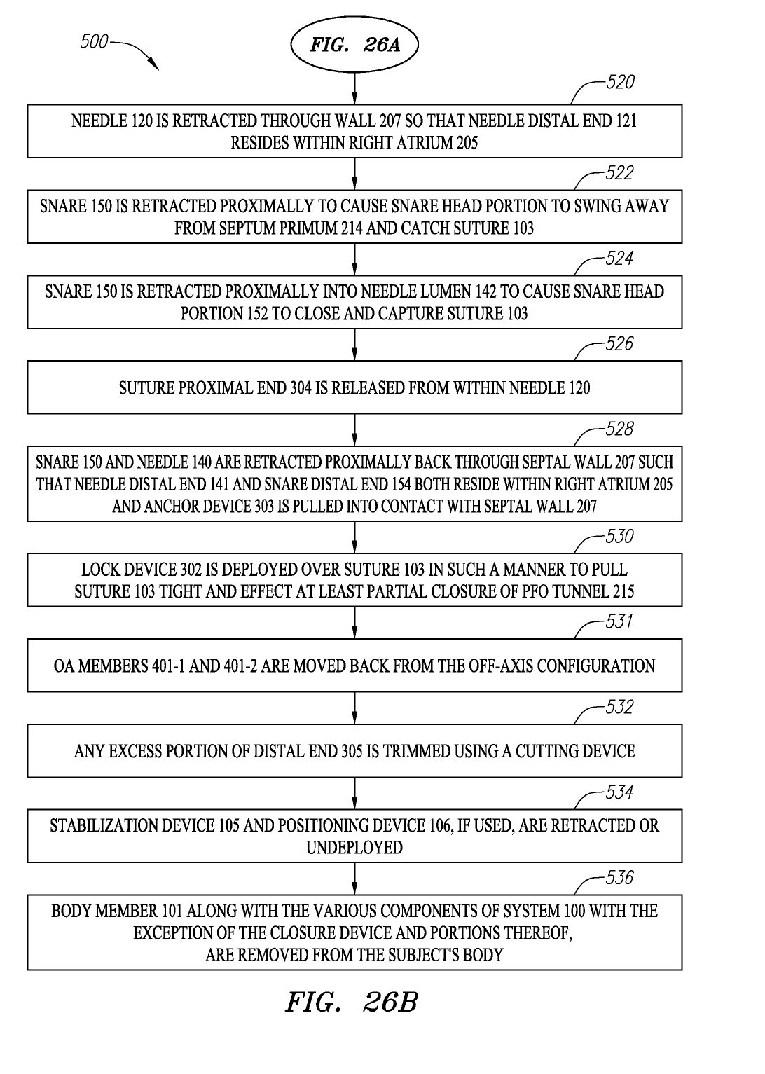

FIGS. 26A-B are flow diagrams depicting an exemplary method 500 of using system 100, implemented in a dual-needle configuration with off-axis capability, to at least partially close a septal defect, such as PFO tunnel 215. It should be noted that this is but one of many different methods that can be used to employ system 100 to close tunnel 215 and that the systems, devices and methods described herein are not limited to this one example. Also, steps 502-536 are not limited to the order in which they are described below and can be performed in different orders if desired. Furthermore, many of the steps below are optional, whether or not described as such, and can be excluded as desired.

At any point during method 500, imaging devices can be used to track progress and aid in completion of the closure procedure. First, at 502, a guidewire is advanced through the subject's vasculature, right atrium 205, tunnel 215 and into left atrium 212. The distal end of the guidewire is optionally advanced into the pulmonary vein or any other vasculature to act as an anchor. At 504, body member 101 is advanced along the guidewire into right atrium 205 and then, if desired, through tunnel 215 and into left atrium 212. The use of body member 101 in conjunction with guidewires is discussed in further detail in the incorporated co-pending application entitled “Systems and Methods for Treating Septal Defects” (Ser. No. 11/175,814). If stabilization device 105 and positioning device 106 are used, body member 101 is preferably advanced into tunnel 215 to allow for the deployment of devices 105 and 106. Devices 105 and 106 are also discussed in further detail in the incorporated co-pending application entitled “Systems and Methods for Treating Septal Defects” (Ser. No. 11/175,814). Then, at 506, devices 105 and 106 are be deployed.

Next, at 508, OA members 401-1 and 401-2 are moved as appropriate to enter the off-axis configuration. At 510, needle 140 is advanced from within lumen 402-2 and through septal wall 207 at location 133 to create opening 137. Then, at 512, snare 150 is advanced distally from within needle lumen 142 to allow snare head portion 152 to enter the open configuration and deflect back towards septal wall 207. At 514, needle 140 and snare 150 are optionally retracted proximally to bring snare head portion 152 into contact with septum primum 214, preferably in a position that encompasses the desired penetration location 132 of needle 120.

Once snare head portion 152 is in position, at 516, needle 120 is preferably advanced through septal wall 207 at location 132 to create opening 136. Next, at 518, suture distal end 305 is deployed from within needle lumen 122. Then, at 520, needle 120 is retracted through septal wall 207 so that needle distal end 121 resides within right atrium 205. At 522, snare 150 is retracted proximally to cause snare head portion to swing away from septum primum 214 and catch suture 103. At 524, snare 150 is retracted proximally into needle lumen 142 to cause snare head portion 152 to close and capture suture 103. At 526, suture proximal end 304 is released from within needle 120. Then, at 528, snare 150 and needle 140 are retracted proximally back through septal wall 207 such that needle distal end 141 and snare distal end 156 both reside within right atrium 205 and anchor device 303, located on suture proximal end 304, is pulled into contact with septal wall 207.

At 530, lock device 302 is deployed over suture 103 in such a manner to pull suture 103 tight and effect at least partial closure, and preferably fall closure, of PFO tunnel 215. Next, at 531, OA members 401-1 and 401-2 can be moved back from the off-axis configuration. At 532, any excess portion of distal end 305 can be trimmed using a cutting device. At this point, the implantation suture 103 has been completed and, at 534, stabilization device 105 and positioning device 106, if used, are retracted or undeployed. Finally, at 536, body member 101 along with the various components of system 100 with the exception of the implanted suture 103, are be removed from the subject's body.

It should be noted that any feature, function, method or component of any embodiment described with respect to FIGS. 1-26B can be used in combination with any other embodiment, whether or not described herein. As one of skill in the art will readily recognize, treatment system 100 and the methods for treating a septal defect can be configured or altered in an almost limitless number of ways, the many combinations and variations of which cannot be practically described herein.

The devices and methods herein may be used in any part of the body, in order to treat a variety of disease states. Of particular interest are applications within hollow organs including but not limited to the heart and blood vessels (arterial and venous), lungs and air passageways, digestive organs (esophagus, stomach, intestines, biliary tree, etc.). The devices and methods will also find use within the genitourinary tract in such areas as the bladder, urethra, ureters, and other areas.

Other locations in which and around which the subject devices and methods find use include the liver, spleen, pancreas and kidney. Any thoracic, abdominal, pelvic, or intravascular location falls within the scope of this description.

The devices and methods may also be used in any region of the body in which it is desirable to appose tissues. This may be useful for causing apposition of the skin or its layers (dermis, epidermis, etc), fascia, muscle, peritoneum, and the like. For example, the subject devices may be used after laparoscopic and/or thoracoscopic procedures to close trocar defects, thus minimizing the likelihood of subsequent hernias. Alternatively, devices that can be used to tighten or lock sutures may find use in various laparoscopic or thoracoscopic procedures where knot tying is required, such as bariatric procedures (gastric bypass and the like) and Nissen fundoplication. The subject devices and methods may also be used to close vascular access sites (either percutaneous, or cut-down). These examples are not meant to be limiting.

The devices and methods can also be used to apply various patch-like or non-patchlike implants (including but not limited to Dacron, Marlex, surgical meshes, and other synthetic and non-synthetic materials) to desired locations. For example, the subject devices may be used to apply mesh to facilitate closure of hernias during open, minimally invasive, laparoscopic, and preperitoneal surgical hernia repairs.

The systems and methods described herein are not limited to use with catheter-based intravascular systems and can be used in conjunction with surgical procedures such as open-heart and procedures based from a port in the chest, or any other procedure where the purpose is to treat a septal defect.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit of the disclosure. It should also be noted that the features described with regard to any embodiment or any particular figure can be freely combined with other embodiments without explicitly stating such.

What is claimed is:

1. A method for treating a patent foramen ovale (PFO), comprising:
passing a first portion of a closure element from a first atrium of a subject through a first opening in a septal wall into a second atrium of the subject;
capturing the first portion of the closure element with a capture device;
passing the first portion of the closure element from the second atrium through a second opening in the septal wall into the first atrium; and
at least partially closing the PFO with the closure element.

2. The method of claim 1, wherein the capture device comprises an elongate, flexible body, the method further comprising:
inserting a first elongate tubular member having a substantially sharp distal end and an inner lumen through the septal wall to create the second opening prior to capturing the closure element, wherein the inner lumen of the first elongate tubular member has an open distal end and is configured to slidably receive the capture device.

3. The method of claim 2, wherein the capture device is a snare-like device, the method further comprising:
advancing a looped portion of the snare-like device distally through the open distal end, the looped portion being configured to capture the closure element.

4. The method of claim 3, wherein the looped portion of the snare-like device is deformable between a closed configuration and an open configuration and biased towards the open configuration.

5. The method of claim 4, wherein the looped portion transitions from the closed configuration to the open configuration upon advancement of the looped portion through the open distal end of the elongate tubular member.

6. The method of claim 5, wherein the looped portion deflects from a substantially undeflected position into a position substantially parallel with the septal wall upon advancement of the looped portion through the open distal end.

7. The method of claim 6, wherein the looped portion comprises a distal wedge-shaped catch portion and a proximal tapered portion.

8. The method of claim 7, wherein the surface of the wedge-shaped catch portion is textured.

9. The method of claim 7, wherein the proximal tapered portion is configured to facilitate transition of the looped portion from the open configuration to the closed configuration.

10. The method of claim 6, wherein the snare body in the looped portion is coiled.

11. The method of claim 6, wherein passing the first portion of the closure element from the first atrium of the subject through the first opening in the septal wall into a second atrium of the subject comprises:
inserting a second elongate tubular member having an open, substantially sharp distal end and an inner lumen through the septal wall to create the first opening, wherein the inner lumen of the second elongate tubular member is configured to house the closure element.

12. The method of claim 11, further comprising:
advancing the first portion of the closure element from within the inner lumen of the second elongate member such that at least part of the first portion of the closure element is within the looped portion of the snare-like device.

13. The method of claim 12, wherein capturing the first portion of the closure element with the snare-like device comprises proximally retracting the snare-like device such that the looped portion deflects back to the substantially undeflected position and captures the first portion of the closure element.

14. The method of claim 13, wherein capturing the first portion of the closure element with the snare-like device further comprises proximally retracting the snare-like device such that the looped portion transitions back to the closed configuration.

15. The method of claim 13, wherein capturing the first portion of the closure element with the snare-like device further comprises distally advancing a collet over the looped portion of the snare-like device such that the looped portion transitions back to the closed configuration.

16. The method of claim 14, wherein passing the first portion of the closure element from the second atrium through the second opening in the septal wall into the first atrium comprises proximally retracting the snare-like device and first elongate member from the second atrium through the second opening in the septal wall into the first atrium with the captured first portion of the closure element.

17. The method of claim 16, further comprising:
releasing a proximal end of the closure element from the second elongate member, the proximal end comprising an anchor device, and proximally retracting the snare-like device to at least partially close the tunnel.

18. The method of claim 17, further comprising:
placing a lock device over the part of the first portion of the closure element exposed in the first atrium.

19. The method of claim 18, further comprising:
placing a lock device over the part of the first portion of the closure element exposed in the first atrium and a second portion of the closure element extending between the second elongate member and the first opening.

20. The method of claim 11, further comprising:
positioning the first and second elongate members in an off-axis orientation prior to inserting the first and second elongate members through the septal wall.

21. The method of claim 20, wherein the first elongate tubular member is slidably disposed within an inner lumen of a third elongate tubular member having an open distal end and the second elongate tubular member is slidably disposed within an inner lumen of a fourth elongate tubular member having an open distal end, the third and fourth elongate tubular members being slidably disposed within an inner lumen of an elongate body member having an open distal end.

22. The method of claim 21, wherein the distal ends of the third and fourth elongate members are coupled to an orientation device that is coupled to an elongate support portion of the body member.

23. The method of claim 22, wherein positioning the first and second elongate members in an off-axis orientation comprises distally advancing the third and fourth elongate members such that the orientation device moves the distal ends of the third and fourth elongate members apart.

24. The method of claim 22, wherein positioning the first and second elongate members in an off-axis orientation comprises distally advancing the third and fourth elongate members such that the orientation device moves the distal ends of the third and fourth elongate members apart and faces the distal ends of the third and fourth elongate members towards the septal wall.

25. The method of claim 24, wherein the orientation device comprises at least one pivot member coupled with the third elongate member by way of a first arm member and the fourth elongate member by way of a second arm member.

26. The method of claim 1, wherein the closure element comprises a distal end configured to facilitate capture by the capture device.

27. The method of claim 26, wherein the distal end of the closure element is curved.

28. The method of claim 26, wherein the distal end of the closure element is bent.

29. The method of claim 1, wherein a distal portion of the closure element is configured to facilitate capture by the capture device.

30. The method of claim 29, wherein the distal portion of the closure element comprises a plurality of protrusions.

31. The method of claim 29, wherein the plurality of protrusions are located at different positions along the length of the closure element.

32. The method of claim 29, wherein the distal portion of the closure element is configured in a serpentine-like manner.

33. The method of claim 29, wherein the distal portion of the closure element is configured in a coil-like manner.

34. The method of claim 29, wherein the distal portion of the closure element is configured in a sawtooth-like manner.

35. The method of claim 29, wherein the closure element is configured as a suture.

36. The method of claim 1, wherein the closure element has a substantially sharp distal end and wherein passing the first portion of the closure element from the first atrium of the subject through the first opening in the septal wall into a second atrium of the subject comprises:
inserting the distal end of the closure element through the septal wall to create the first opening.

37. The method of claim 36, wherein the capture device has a substantially sharp distal end and wherein passing the first portion of the closure element from the second atrium through the second opening in the septal wall into the first atrium comprises:
inserting the distal end of the capture device through the septal wall to create the second opening.

38. The method of claim 1, wherein the capture device has a substantially sharp distal end and wherein passing the first portion of the closure element from the second atrium through the second opening in the septal wall into the first atrium comprises:
inserting the distal end of the capture device through the septal wall to create the second opening.

39. The method of claim 1, wherein the capture device comprises an elongate, flexible body, the method further comprising:
inserting a first elongate tubular member having a substantially sharp distal end and an inner lumen through the septal wall to create the second opening prior to passing a first portion of a closure element from the first atrium of the subject through a first opening in a septal wall into the second atrium of the subject, wherein the inner lumen of the first elongate tubular member has an open distal end and is configured to slidably receive the capture device.

40. The method of claim 39, wherein the first elongate tubular member is advanced through the second opening while contained within the inner lumen of an outer tubular member having an open distal end.

41. The method of claim 39, wherein a distal portion of the first elongate tubular member is deformable between a substantially straight configuration and a curved configuration.

42. The method of claim 40, wherein the distal portion of the first elongate tubular member is biased towards the curved configuration.

43. The method of claim 42, further comprising:
advancing the first elongate member through the open distal end of the outer tubular member to allow the distal portion of the first elongate member to transition to the curved configuration.

44. The method of claim 43, further comprising:
proximally retracting the first elongate member while in the curved configuration to create the first opening in the septal wall.

45. The method of claim 44, wherein capturing the closure element occurs prior to passing the first portion of the closure element through the septal wall.

46. The method of claim 45, wherein the capture device is a snare-like device, the method further comprising:
advancing a looped portion of the snare-like device distally through the open distal end of the first elongate member prior to passing the first portion of the closure element through the first opening, the looped portion being configured to capture the closure element.

47. The method of claim 46, further comprising advancing the first portion of the closure element through an open distal end of a second elongate tubular member having an inner lumen, wherein the first portion of the closure element is positioned to allow capture by the snare-like device.

48. The method of claim 47, wherein passing a first portion of a closure element from a first atrium of a subject through a first opening in a septal wall into a second atrium of the subject comprises:
proximally retracting the looped portion of the snare-like device into the inner lumen of the first elongate member after capturing the closure element in the looped portion; and
distally advancing the first elongate member through the first opening into the second atrium with the captured first portion of the closure element.

49. The method of claim 48, wherein passing at least a part of the first portion of the closure element from the second atrium through the second opening in the septal wall into the first atrium comprises:
proximally retracting the first elongate member into the inner lumen of the outer tubular member such that the curved distal portion of the first elongate member transitions back to the substantially straight configuration; and
proximally retracting the first elongate member and outer tubular member back through the second opening into the first atrium with the captured first portion of the closure element.

50. The method of claim 49, further comprising:
releasing a proximal end of the closure element from the second elongate member, the proximal end comprising an anchor device, and proximally retracting the snare-like device to at least partially close the tunnel.

51. The method of claim 50, further comprising:
placing a lock device over the part of the first portion of the closure element exposed in the first atrium.

52. The method of claim 49, further comprising:
placing a lock device over the part of the first portion of the closure element exposed in the first atrium and a second portion of the closure element extending between the second elongate member and the first opening.

53. The method of claim 1, wherein the first atrium is a right atrium and the second atrium is a left atrium.

54. The method of claim 1, wherein the first atrium is a left atrium and the second atrium is a right atrium.

55. The method of claim 1, wherein at least partially closing the PFO with the closure element comprises closing the PFO to a degree that prevents a substantial amount of blood from passing through the PFO.
